SEQUENCE LISTING (256 sequences)

SEQ ID NO: 1 - RNA, 23 nt, synthetic construct
  source 1..23: mol_type = other RNA
attagcagaactacacaccaggg

SEQ ID NO: 2 - RNA, 23 nt, synthetic construct
  source 1..23: mol_type = other RNA
attggtcttaaaggtacctgagg

SEQ ID NO: 3 - RNA, 23 nt, synthetic construct
  source 1..23: mol_type = other RNA
cagggaagtagccttgtgtgtgg

SEQ ID NO: 4 - RNA, 23 nt, synthetic construct
  source 1..23: mol_type = other RNA
tgcctggctagaagcacaagagg

SEQ ID NO: 5 - RNA, 23 nt, synthetic construct
  source 1..23: mol_type = other RNA
ggattttgctataagatgggtgg

SEQ ID NO: 6 - RNA, 23 nt, synthetic construct
  source 1..23: mol_type = other RNA
ctgtggatctaccacacacaagg

SEQ ID NO: 7 - RNA, 23 nt, synthetic construct
  source 1..23: mol_type = other RNA
gacaagatatccttgatctgtgg

SEQ ID NO: 8 - RNA, 23 nt, synthetic construct
  source 1..23: mol_type = other RNA
gcacaagaggaggaggaggtggg

SEQ ID NO: 9 - RNA, 23 nt, synthetic construct
  source 1..23: mol_type = other RNA
cactttttaaaagaaaagggggg

SEQ ID NO: 10 - RNA, 23 nt, synthetic construct
  source 1..23: mol_type = other RNA
caaaaagtagtgtgattggatgg

SEQ ID NO: 11 - RNA, 23 nt, synthetic construct
  source 1..23: mol_type = other RNA
gctataagatgggtggcaagtgg

SEQ ID NO: 12 - RNA, 23 nt, synthetic construct
  source 1..23: mol_type = other RNA
ctggctagaagcacaagaggagg

SEQ ID NO: 13 - RNA, 23 nt, synthetic construct
  source 1..23: mol_type = other RNA
gattagcagaactacacaccagg

SEQ ID NO: 14 - RNA, 23 nt, synthetic construct
  source 1..23: mol_type = other RNA
gagccagcagcagatagggtggg

SEQ ID NO: 15 - RNA, 23 nt, synthetic construct
  source 1..23: mol_type = other RNA
ccactttttaaaagaaaaggggg

SEQ ID NO: 16 - RNA, 23 nt, synthetic construct
  source 1..23: mol_type = other RNA
aagaaaaggggggactggaaggg

SEQ ID NO: 17 - RNA, 23 nt, synthetic construct
  source 1..23: mol_type = other RNA
tgtggtagatccacagatcaagg

SEQ ID NO: 18 - RNA, 23 nt, synthetic construct
  source 1..23: mol_type = other RNA
gattggatggcctactgtaaggg

SEQ ID NO: 19 - RNA, 23 nt, synthetic construct
  source 1..23: mol_type = other RNA
tctcgagacctggaaaaacatgg

SEQ ID NO: 20 - RNA, 23 nt, synthetic construct
  source 1..23: mol_type = other RNA
acagctgccttgtaagtcattgg

SEQ ID NO: 21 - RNA, 28 nt, synthetic construct
  source 1..28: mol_type = other RNA
ttccaggtctcgagatgctgctcccacc

SEQ ID NO: 22 - RNA, 28 nt, synthetic construct
  source 1..28: mol_type = other RNA
ttctagccaggcacaagcagcattggta

SEQ ID NO: 23 - RNA, 28 nt, synthetic construct
  source 1..28: mol_type = other RNA
ttcttttaaaaagtggctaagatctaca

SEQ ID NO: 24 - RNA, 28 nt, synthetic construct
  source 1..28: mol_type = other RNA
ttccagtccccccttttcttttaaaaag

SEQ ID NO: 25 - RNA, 28 nt, synthetic construct
  source 1..28: mol_type = other RNA
ttctttgggagtgaattagcccttccag

SEQ ID NO: 26 - RNA, 28 nt, synthetic construct
  source 1..28: mol_type = other RNA
ttcactcccaaagaagacaagatatcct

SEQ ID NO: 27 - RNA, 28 nt, synthetic construct
  source 1..28: mol_type = other RNA
ttccctgattagcagaactacacaccag

SEQ ID NO: 28 - RNA, 28 nt, synthetic construct
  source 1..28: mol_type = other RNA
ttcccttacagtaggccatccaatcaca

SEQ ID NO: 29 - RNA, 28 nt, synthetic construct
  source 1..28: mol_type = other RNA
ttctttcccttacagtaggccatccaat

SEQ ID NO: 30 - RNA, 28 nt, synthetic construct
  source 1..28: mol_type = other RNA
ttccagtcacacctcaggtacctttaag

SEQ ID NO: 31 - RNA, 28 nt, synthetic construct
  source 1..28: mol_type = other RNA
ttctgctaatcagggaagtagccttgtg

SEQ ID NO: 32 - RNA, 23 nt, synthetic construct
  source 1..23: mol_type = other RNA
gcatgggtaccagcacacaaagg

SEQ ID NO: 33 - RNA, 23 nt, synthetic construct
  source 1..23: mol_type = other RNA
gtataaacaatgagacaccaggg

SEQ ID NO: 34 - RNA, 23 nt, synthetic construct
  source 1..23: mol_type = other RNA
ctcccactcaggaatccaggtgg

SEQ ID NO: 35 - RNA, 23 nt, synthetic construct
  source 1..23: mol_type = other RNA
tgatgtaaaacaattaacagagg

SEQ ID NO: 36 - RNA, 23 nt, synthetic construct
  source 1..23: mol_type = other RNA
gggcaagtcagatttacccaggg

SEQ ID NO: 37 - RNA, 23 nt, synthetic construct
  source 1..23: mol_type = other RNA
cgttgccaaagagtgacctgagg

SEQ ID NO: 38 - RNA, 23 nt, synthetic construct
  source 1..23: mol_type = other RNA
tcgtcacaataaagatagggggg

SEQ ID NO: 39 - RNA, 23 nt, synthetic construct
  source 1..23: mol_type = other RNA
gatggcaggtgatgattgtgtgg

SEQ ID NO: 40 - RNA, 23 nt, synthetic construct
  source 1..23: mol_type = other RNA
aggaaacatgggaaacatggtgg

SEQ ID NO: 41 - protein, 23 aa, synthetic construct
GTTGCCAAAGAGTGACCTGAGGG

SEQ ID NO: 42 - protein, 23 aa, synthetic construct
GGTGATCCTTTCCATCCCTGTGG

SEQ ID NO: 43 - RNA, 23 nt, synthetic construct
  source 1..23: mol_type = other RNA
attctaaaagaaccagtacatgg

SEQ ID NO: 44 - RNA, 23 nt, synthetic construct
  source 1..23: mol_type = other RNA
tggaaaccaaaaatgataggggg

SEQ ID NO: 45 - RNA, 23 nt, synthetic construct
  source 1..23: mol_type = other RNA
tatcggctcctgcttctgagggg

SEQ ID NO: 46 - RNA, 23 nt, synthetic construct
  source 1..23: mol_type = other RNA
acagaaagcatagtaatatgggg

SEQ ID NO: 47 - RNA, 23 nt, synthetic construct
  source 1..23: mol_type = other RNA
aagccacctggattcctgagtgg

SEQ ID NO: 48 - RNA, 23 nt, synthetic construct
  source 1..23: mol_type = other RNA
acaggatgaggattagaacatgg

SEQ ID NO: 49 - RNA, 23 nt, synthetic construct
  source 1..23: mol_type = other RNA
agaaaccttctatgtagatgggg

SEQ ID NO: 50 - RNA, 23 nt, synthetic construct
  source 1..23: mol_type = other RNA
cagatactcatagaaatctgtgg

SEQ ID NO: 51 - RNA, 23 nt, synthetic construct
  source 1..23: mol_type = other RNA
acaattttaaaagaaaagggggg

SEQ ID NO: 52 - RNA, 28 nt, synthetic construct
  source 1..28: mol_type = other RNA
ttctaagtcagatcctacatacaaatca

SEQ ID NO: 53 - RNA, 28 nt, synthetic construct
  source 1..28: mol_type = other RNA
ttctatgctgccctatttctaagtcaga

SEQ ID NO: 54 - RNA, 28 nt, synthetic construct
  source 1..28: mol_type = other RNA
ttctgatgttttttgtctggtgtggtaa

SEQ ID NO: 55 - RNA, 28 nt, synthetic construct
  source 1..28: mol_type = other RNA
ttctttctgatgttttttgtctggtgtg

SEQ ID NO: 56 - RNA, 28 nt, synthetic construct
  source 1..28: mol_type = other RNA
ttccctaaaaaattagcctgtctctcag

SEQ ID NO: 57 - RNA, 28 nt, synthetic construct
  source 1..28: mol_type = other RNA
ttccccactaacttctgtatgtcattga

SEQ ID NO: 58 - RNA, 28 nt, synthetic construct
  source 1..28: mol_type = other RNA
ttcaatttccccactaacttctgtatgt

SEQ ID NO: 59 - RNA, 28 nt, synthetic construct
  source 1..28: mol_type = other RNA
ttctgagggggagttgttgtctctaccc

SEQ ID NO: 60 - RNA, 28 nt, synthetic construct
  source 1..28: mol_type = other RNA
ttctgttagtgctttggttcctctaagg

SEQ ID NO: 61 - RNA, 28 nt, synthetic construct
  source 1..28: mol_type = other RNA
ttctgttagtggtattacttctgttagt

SEQ ID NO: 62 - RNA, 28 nt, synthetic construct
  source 1..28: mol_type = other RNA
ttcttctgttagtggtattacttctgtt

SEQ ID NO: 63 - RNA, 28 nt, synthetic construct
  source 1..28: mol_type = other RNA
ttctagctctgcttcttctgttagtggt

SEQ ID NO: 64 - RNA, 28 nt, synthetic construct
  source 1..28: mol_type = other RNA
ttctgccagttctagctctgcttcttct

SEQ ID NO: 65 - RNA, 28 nt, synthetic construct
  source 1..28: mol_type = other RNA
ttcttttagaatctctctgttttctgcc

SEQ ID NO: 66 - RNA, 28 nt, synthetic construct
  source 1..28: mol_type = other RNA
ttctaaaagaaccagtacatggagtgta

SEQ ID NO: 67 - RNA, 28 nt, synthetic construct
  source 1..28: mol_type = other RNA
ttctgctattaagtcttttgatgggtca

SEQ ID NO: 68 - RNA, 28 nt, synthetic construct
  source 1..28: mol_type = other RNA
ttctgtatttctgctattaagtcttttg

SEQ ID NO: 69 - RNA, 28 nt, synthetic construct
  source 1..28: mol_type = other RNA
ttccttgtctatcggctcctgcttctga

SEQ ID NO: 70 - RNA, 28 nt, synthetic construct
  source 1..28: mol_type = other RNA
ttcagatttttaaatggctcttgataaa

SEQ ID NO: 71 - RNA, 28 nt, synthetic construct
  source 1..28: mol_type = other RNA
ttcctgttttcagatttttaaatggctc

SEQ ID NO: 72 - RNA, 23 nt, synthetic construct
  source 1..23: mol_type = other RNA
acttactcttgattgtaacgagg

SEQ ID NO: 73 - RNA, 23 nt, synthetic construct
  source 1..23: mol_type = other RNA
tcaagagtaagtctctcaagcgg

SEQ ID NO: 74 - RNA, 23 nt, synthetic construct
  source 1..23: mol_type = other RNA
gaaggaatagaagaagaaggtgg

SEQ ID NO: 75 - RNA, 23 nt, synthetic construct
  source 1..23: mol_type = other RNA
agatccattcgattagtgaacgg

SEQ ID NO: 76 - RNA, 23 nt, synthetic construct
  source 1..23: mol_type = other RNA
acccacctcccaaccccgagggg

SEQ ID NO: 77 - RNA, 23 nt, synthetic construct
  source 1..23: mol_type = other RNA
atcgtcccagataagtgccaagg

SEQ ID NO: 78 - RNA, 23 nt, synthetic construct
  source 1..23: mol_type = other RNA
agacccacctcccaaccccgagg

SEQ ID NO: 79 - RNA, 23 nt, synthetic construct
  source 1..23: mol_type = other RNA
agagtaagtctctcaagcggtgg

SEQ ID NO: 80 - RNA, 23 nt, synthetic construct
  source 1..23: mol_type = other RNA
tggaacttctgggacgcaggggg

SEQ ID NO: 81 - RNA, 23 nt, synthetic construct
  source 1..23: mol_type = other RNA
gggcctgtcgggtcccctcgggg

SEQ ID NO: 82 - RNA, 23 nt, synthetic construct
  source 1..23: mol_type = other RNA
cttgattgtaacgaggattgtgg

SEQ ID NO: 83 - RNA, 23 nt, synthetic construct
  source 1..23: mol_type = other RNA
gtggaacttctgggacgcagggg

SEQ ID NO: 84 - RNA, 23 nt, synthetic construct
  source 1..23: mol_type = other RNA
tcaagcggtggtagctgaagagg

SEQ ID NO: 85 - RNA, 23 nt, synthetic construct
  source 1..23: mol_type = other RNA
ggatccgttcactaatcgaatgg

SEQ ID NO: 86 - RNA, 23 nt, synthetic construct
  source 1..23: mol_type = other RNA
gttcctgactccaatactgtagg

SEQ ID NO: 87 - RNA, 23 nt, synthetic construct
  source 1..23: mol_type = other RNA
ggtggaatctcctacagtattgg

SEQ ID NO: 88 - RNA, 23 nt, synthetic construct
  source 1..23: mol_type = other RNA
gacccacctcccaaccccgaggg

SEQ ID NO: 89 - RNA, 23 nt, synthetic construct
  source 1..23: mol_type = other RNA
aggggacccgacaggcccgaagg

SEQ ID NO: 90 - RNA, 23 nt, synthetic construct
  source 1..23: mol_type = other RNA
cccgaaggaatagaagaagaagg

SEQ ID NO: 91 - RNA, 23 nt, synthetic construct
  source 1..23: mol_type = other RNA
gggaagccctcaaatattggtgg

SEQ ID NO: 92 - RNA, 28 nt, synthetic construct
  source 1..28: mol_type = other RNA
ttcagctaccaccgcttgagagacttac

SEQ ID NO: 93 - RNA, 28 nt, synthetic construct
  source 1..28: mol_type = other RNA
ttccacaatcctcgttacaatcaagagt

SEQ ID NO: 94 - RNA, 28 nt, synthetic construct
  source 1..28: mol_type = other RNA
ttcccaccccctgcgtcccagaagttcc

SEQ ID NO: 95 - RNA, 28 nt, synthetic construct
  source 1..28: mol_type = other RNA
ttccaccaatatttgagggcttcccacc

SEQ ID NO: 96 - RNA, 28 nt, synthetic construct
  source 1..28: mol_type = other RNA
ttctttagttcctgactccaatactgta

SEQ ID NO: 97 - RNA, 28 nt, synthetic construct
  source 1..28: mol_type = other RNA
ttcgggcctgtcgggtcccctcggggtt

SEQ ID NO: 98 - RNA, 28 nt, synthetic construct
  source 1..28: mol_type = other RNA
ttccttcgggcctgtcgggtcccctcgg

SEQ ID NO: 99 - RNA, 28 nt, synthetic construct
  source 1..28: mol_type = other RNA
ttctattccttcgggcctgtcgggtccc

SEQ ID NO: 100 - RNA, 28 nt, synthetic construct
  source 1..28: mol_type = other RNA
ttcttctattccttcgggcctgtcgggt

SEQ ID NO: 101 - RNA, 28 nt, synthetic construct
  source 1..28: mol_type = other RNA
ttcttcttctattccttcgggcctgtcg

SEQ ID NO: 102 - RNA, 28 nt, synthetic construct
  source 1..28: mol_type = other RNA
ttcaccattatcgtttcagacccacctc

SEQ ID NO: 103 - RNA, 28 nt, synthetic construct
  source 1..28: mol_type = other RNA
ttcactaatcgaatggatctgtctctgt

SEQ ID NO: 104 - RNA, 28 nt, synthetic construct
  source 1..28: mol_type = other RNA
ttcgattagtgaacggatccttggcact

SEQ ID NO: 105 - RNA, 28 nt, synthetic construct
  source 1..28: mol_type = other RNA
ttctgggacgcagggggtgggaagccct

SEQ ID NO: 106 - RNA, 28 nt, synthetic construct
  source 1..28: mol_type = other RNA
ttcagacccacctcccaaccccgagggg

SEQ ID NO: 107 - RNA, 28 nt, synthetic construct
  source 1..28: mol_type = other RNA
ttcctgactccaatactgtaggagattc

SEQ ID NO: 108 - RNA, 23 nt, synthetic construct
  source 1..23: mol_type = other RNA
gctaccataatgatgcagagagg

SEQ ID NO: 109 - RNA, 23 nt, synthetic construct
  source 1..23: mol_type = other RNA
aaacaccatgctaaacacagtgg

SEQ ID NO: 110 - RNA, 23 nt, synthetic construct
  source 1..23: mol_type = other RNA
agcattctggacataagacaagg

SEQ ID NO: 111 - RNA, 23 nt, synthetic construct
  source 1..23: mol_type = other RNA
gagagcgtcagtattaagcgggg

SEQ ID NO: 112 - RNA, 23 nt, synthetic construct
  source 1..23: mol_type = other RNA
gttaaaagagaccatcaatgagg

SEQ ID NO: 113 - RNA, 23 nt, synthetic construct
  source 1..23: mol_type = other RNA
ggccagatgagagaaccaagggg

SEQ ID NO: 114 - RNA, 23 nt, synthetic construct
  source 1..23: mol_type = other RNA
aaaattcggttaaggccaggggg

SEQ ID NO: 115 - RNA, 23 nt, synthetic construct
  source 1..23: mol_type = other RNA
ctatagtgcagaacatccagggg

SEQ ID NO: 116 - RNA, 23 nt, synthetic construct
  source 1..23: mol_type = other RNA
agagcgtcagtattaagcggggg

SEQ ID NO: 117 - RNA, 23 nt, synthetic construct
  source 1..23: mol_type = other RNA
cgttgccaaagagtgacctgagg

SEQ ID NO: 118 - RNA, 23 nt, synthetic construct
  source 1..23: mol_type = other RNA
tcgtcacaataaagatagggggg

SEQ ID NO: 119 - RNA, 23 nt, synthetic construct
  source 1..23: mol_type = other RNA
gaaggctgtagacaaatactggg

SEQ ID NO: 120 - RNA, 23 nt, synthetic construct
  source 1..23: mol_type = other RNA
accatgctaaacacagtgggggg

SEQ ID NO: 121 - RNA, 23 nt, synthetic construct
  source 1..23: mol_type = other RNA
ggcctgttagaaacatcagaagg

SEQ ID NO: 122 - RNA, 23 nt, synthetic construct
  source 1..23: mol_type = other RNA
agccgagcaagcttcacaggagg

SEQ ID NO: 123 - RNA, 23 nt, synthetic construct
  source 1..23: mol_type = other RNA
caggccagatgagagaaccaagg

SEQ ID NO: 124 - RNA, 23 nt, synthetic construct
  source 1..23: mol_type = other RNA
gttgccaaagagtgacctgaggg

SEQ ID NO: 125 - RNA, 23 nt, synthetic construct
  source 1..23: mol_type = other RNA
ggacataagacaaggaccaaagg

SEQ ID NO: 126 - RNA, 23 nt, synthetic construct
  source 1..23: mol_type = other RNA
tatcggctcctgcttctgagggg

SEQ ID NO: 127 - RNA, 23 nt, synthetic construct
  source 1..23: mol_type = other RNA
gggctgttggaaatgtggaaagg

SEQ ID NO: 128 - RNA, 28 nt, synthetic construct
  source 1..28: mol_type = other RNA
ttctagtgtagccgctggtcccaatgct

SEQ ID NO: 129 - RNA, 28 nt, synthetic construct
  source 1..28: mol_type = other RNA
ttcttctagtgtagccgctggtcccaat

SEQ ID NO: 130 - RNA, 28 nt, synthetic construct
  source 1..28: mol_type = other RNA
ttcagccaaaactcttgccttatggccg

SEQ ID NO: 131 - RNA, 28 nt, synthetic construct
  source 1..28: mol_type = other RNA
ttcagctaccataatgatgcagagaggc

SEQ ID NO: 132 - RNA, 28 nt, synthetic construct
  source 1..28: mol_type = other RNA
ttcctaaaattgcctctctgcatcatta

SEQ ID NO: 133 - RNA, 28 nt, synthetic construct
  source 1..28: mol_type = other RNA
ttctttggttcctaaaattgcctctctg

SEQ ID NO: 134 - RNA, 28 nt, synthetic construct
  source 1..28: mol_type = other RNA
ttctttgccacaattgaaacacttaaca

SEQ ID NO: 135 - RNA, 28 nt, synthetic construct
  source 1..28: mol_type = other RNA
ttctggctgtgtgcccttctttgccaca

SEQ ID NO: 136 - RNA, 28 nt, synthetic construct
  source 1..28: mol_type = other RNA
ttccacatttccaacagccctttttcct

SEQ ID NO: 137 - RNA, 28 nt, synthetic construct
  source 1..28: mol_type = other RNA
ttcctttccacatttccaacagcccttt

SEQ ID NO: 138 - RNA, 28 nt, synthetic construct
  source 1..28: mol_type = other RNA
ttctagctccctgcttgcccatactata

SEQ ID NO: 139 - RNA, 28 nt, synthetic construct
  source 1..28: mol_type = other RNA
ttcatttggtgtccttcctttccacatt

SEQ ID NO: 140 - RNA, 28 nt, synthetic construct
  source 1..28: mol_type = other RNA
ttccctaaaaaattagcctgtctctcag

SEQ ID NO: 141 - RNA, 28 nt, synthetic construct
  source 1..28: mol_type = other RNA
ttcctacaagggaaggccagggaatttt

SEQ ID NO: 142 - RNA, 28 nt, synthetic construct
  source 1..28: mol_type = other RNA
ttcttcagagcagaccagagccaacagc

SEQ ID NO: 143 - RNA, 28 nt, synthetic construct
  source 1..28: mol_type = other RNA
ttcagagcagaccagagccaacagcccc

SEQ ID NO: 144 - RNA, 28 nt, synthetic construct
  source 1..28: mol_type = other RNA
ttctggtggggctgttggctctggtctg

SEQ ID NO: 145 - RNA, 28 nt, synthetic construct
  source 1..28: mol_type = other RNA
ttctgagggggagttgttgtctctaccc

SEQ ID NO: 146 - RNA, 28 nt, synthetic construct
  source 1..28: mol_type = other RNA
ttccttgtctatcggctcctgcttctga

SEQ ID NO: 147 - RNA, 28 nt, synthetic construct
  source 1..28: mol_type = other RNA
ttccctcaggtcactctttggcaacgac

SEQ ID NO: 148 - RNA, 23 nt, synthetic construct
  source 1..23: mol_type = other RNA
gaaggaatagaagaagaaggtgg

SEQ ID NO: 149 - RNA, 23 nt, synthetic construct
  source 1..23: mol_type = other RNA
acccacctcccaaccccgagggg

SEQ ID NO: 150 - RNA, 23 nt, synthetic construct
  source 1..23: mol_type = other RNA
agacccacctcccaaccccgagg

SEQ ID NO: 151 - RNA, 23 nt, synthetic construct
  source 1..23: mol_type = other RNA
gggcctgtcgggtcccctcgggg

SEQ ID NO: 152 - RNA, 23 nt, synthetic construct
  source 1..23: mol_type = other RNA
gacccacctcccaaccccgaggg

SEQ ID NO: 153 - RNA, 23 nt, synthetic construct
  source 1..23: mol_type = other RNA
aggggacccgacaggcccgaagg

SEQ ID NO: 154 - RNA, 23 nt, synthetic construct
  source 1..23: mol_type = other RNA
cccgaaggaatagaagaagaagg

SEQ ID NO: 155 - RNA, 23 nt, synthetic construct
  source 1..23: mol_type = other RNA
aggtgggtctgaaacgataatgg

SEQ ID NO: 156 - RNA, 23 nt, synthetic construct
  source 1..23: mol_type = other RNA
caaccccgaggggacccgacagg

SEQ ID NO: 157 - RNA, 23 nt, synthetic construct
  source 1..23: mol_type = other RNA
tcccctcggggttgggaggtggg

SEQ ID NO: 158 - RNA, 23 nt, synthetic construct
  source 1..23: mol_type = other RNA
cgggtcccctcggggttgggagg

SEQ ID NO: 159 - RNA, 23 nt, synthetic construct
  source 1..23: mol_type = other RNA
ttctattccttcgggcctgtcgg

SEQ ID NO: 160 - RNA, 23 nt, synthetic construct
  source 1..23: mol_type = other RNA
tctattccttcgggcctgtcggg

SEQ ID NO: 161 - RNA, 23 nt, synthetic construct
  source 1..23: mol_type = other RNA
cgggcctgtcgggtcccctcggg

SEQ ID NO: 162 - RNA, 23 nt, synthetic construct
  source 1..23: mol_type = other RNA
tcgggcctgtcgggtcccctcgg

SEQ ID NO: 163 - RNA, 23 nt, synthetic construct
  source 1..23: mol_type = other RNA
accttcttcttctattccttcgg

SEQ ID NO: 164 - RNA, 23 nt, synthetic construct
  source 1..23: mol_type = other RNA
tgtcgggtcccctcggggttggg

SEQ ID NO: 165 - RNA, 23 nt, synthetic construct
  source 1..23: mol_type = other RNA
ctgtcgggtcccctcggggttgg

SEQ ID NO: 166 - RNA, 23 nt, synthetic construct
  source 1..23: mol_type = other RNA
gtcccctcggggttgggaggtgg

SEQ ID NO: 167 - RNA, 28 nt, synthetic construct
  source 1..28: mol_type = other RNA
ttcgggcctgtcgggtcccctcggggtt

SEQ ID NO: 168 - RNA, 28 nt, synthetic construct
  source 1..28: mol_type = other RNA
ttccttcgggcctgtcgggtcccctcgg

SEQ ID NO: 169 - RNA, 28 nt, synthetic construct
  source 1..28: mol_type = other RNA
ttctattccttcgggcctgtcgggtccc

SEQ ID NO: 170 - RNA, 28 nt, synthetic construct
  source 1..28: mol_type = other RNA
ttcttctattccttcgggcctgtcgggt

SEQ ID NO: 171 - RNA, 28 nt, synthetic construct
  source 1..28: mol_type = other RNA
ttcttcttctattccttcgggcctgtcg

SEQ ID NO: 172 - RNA, 28 nt, synthetic construct
  source 1..28: mol_type = other RNA
ttcaccattatcgtttcagacccacctc

SEQ ID NO: 173 - RNA, 28 nt, synthetic construct
  source 1..28: mol_type = other RNA
ttcagacccacctcccaaccccgagggg

SEQ ID NO: 174 - RNA, 23 nt, synthetic construct
  source 1..23: mol_type = other RNA
attagcagaactacacaccaggg

SEQ ID NO: 175 - RNA, 23 nt, synthetic construct
  source 1..23: mol_type = other RNA
tagcttgtagcaccatccaaagg

SEQ ID NO: 176 - RNA, 23 nt, synthetic construct
  source 1..23: mol_type = other RNA
agaactacacaccagggccaggg

SEQ ID NO: 177 - RNA, 23 nt, synthetic construct
  source 1..23: mol_type = other RNA
cagggaagtagccttgtgtgtgg

SEQ ID NO: 178 - RNA, 23 nt, synthetic construct
  source 1..23: mol_type = other RNA
tcagacccttttagtcagtgtgg

SEQ ID NO: 179 - RNA, 23 nt, synthetic construct
  source 1..23: mol_type = other RNA
ctgtggatctaccacacacaagg

SEQ ID NO: 180 - RNA, 23 nt, synthetic construct
  source 1..23: mol_type = other RNA
accagagtcacacaacagacggg

SEQ ID NO: 181 - RNA, 23 nt, synthetic construct
  source 1..23: mol_type = other RNA
gatatccactgacctttggatgg

SEQ ID NO: 182 - RNA, 23 nt, synthetic construct
  source 1..23: mol_type = other RNA
gacaagatatccttgatctgtgg

SEQ ID NO: 183 - RNA, 23 nt, synthetic construct
  source 1..23: mol_type = other RNA
acactgactaaaagggtctgagg

SEQ ID NO: 184 - RNA, 23 nt, synthetic construct
  source 1..23: mol_type = other RNA
taaccagagagacccagtacagg

SEQ ID NO: 185 - RNA, 23 nt, synthetic construct
  source 1..23: mol_type = other RNA
tagcaccatccaaaggtcagtgg

SEQ ID NO: 186 - RNA, 23 nt, synthetic construct
  source 1..23: mol_type = other RNA
taccagagtcacacaacagacgg

SEQ ID NO: 187 - RNA, 23 nt, synthetic construct
  source 1..23: mol_type = other RNA
gattagcagaactacacaccagg

SEQ ID NO: 188 - RNA, 23 nt, synthetic construct
  source 1..23: mol_type = other RNA
agagagaagtgttagagtggagg

SEQ ID NO: 189 - RNA, 23 nt, synthetic construct
  source 1..23: mol_type = other RNA
cactgactaaaagggtctgaggg

SEQ ID NO: 190 - RNA, 23 nt, synthetic construct
  source 1..23: mol_type = other RNA
ctacaagggactttccgctgggg

SEQ ID NO: 191 - RNA, 23 nt, synthetic construct
  source 1..23: mol_type = other RNA
gctcacagggtgtaacaagctgg

SEQ ID NO: 192 - RNA, 23 nt, synthetic construct
  source 1..23: mol_type = other RNA
cacactacttgaagcactcaagg

SEQ ID NO: 193 - RNA, 23 nt, synthetic construct
  source 1..23: mol_type = other RNA
actctaacacttctctctccggg

SEQ ID NO: 194 - RNA, 28 nt, synthetic construct
  source 1..28: mol_type = other RNA
ttctctggctcaactggtactagcttgt

SEQ ID NO: 195 - RNA, 28 nt, synthetic construct
  source 1..28: mol_type = other RNA
ttcttctaacttctctggctcaactggt

SEQ ID NO: 196 - RNA, 28 nt, synthetic construct
  source 1..28: mol_type = other RNA
ttccatgcaggctcacagggtgtaacaa

SEQ ID NO: 197 - RNA, 28 nt, synthetic construct
  source 1..28: mol_type = other RNA
ttcttgaagtactccggatgcagctctc

SEQ ID NO: 198 - RNA, 28 nt, synthetic construct
  source 1..28: mol_type = other RNA
ttcaagaactgctgacatcgagcttgct

SEQ ID NO: 199 - RNA, 28 nt, synthetic construct
  source 1..28: mol_type = other RNA
ttcaagtagtgtgtgcccgtctgttgtg

SEQ ID NO: 200 - RNA, 28 nt, synthetic construct
  source 1..28: mol_type = other RNA
ttccacactgactaaaagggtctgaggg

SEQ ID NO: 201 - RNA, 28 nt, synthetic construct
  source 1..28: mol_type = other RNA
ttccctgattagcagaactacacaccag

SEQ ID NO: 202 - RNA, 28 nt, synthetic construct
  source 1..28: mol_type = other RNA
ttccgctggggactttccagggaggcgt

SEQ ID NO: 203 - RNA, 28 nt, synthetic construct
  source 1..28: mol_type = other RNA
ttccagggaggcgtggcctgggcgggac

SEQ ID NO: 204 - RNA, 28 nt, synthetic construct
  source 1..28: mol_type = other RNA
ttctgctaatcagggaagtagccttgtg

SEQ ID NO: 205 - RNA, 28 nt, synthetic construct
  source 1..28: mol_type = other RNA
ttctctcctttgttggcttcttctaact

SEQ ID NO: 206 - RNA, 28 nt, synthetic construct
  source 1..28: mol_type = other RNA
ttctaacttctctggctcaactggtact

SEQ ID NO: 207 - RNA, 28 nt, synthetic construct
  source 1..28: mol_type = other RNA
ttcactcccaacgaagacaagatatcct

SEQ ID NO: 208 - RNA, 28 nt, synthetic construct
  source 1..28: mol_type = other RNA
ttctctctccgggtcatccattccatgc

SEQ ID NO: 209 - RNA, 28 nt, synthetic construct
  source 1..28: mol_type = other RNA
ttcatcacatggcccgagagctgcatcc

SEQ ID NO: 210 - RNA, 28 nt, synthetic construct
  source 1..28: mol_type = other RNA
ttccctagttagccagagagctcccagg

SEQ ID NO: 211 - RNA, 23 nt, synthetic construct
  source 1..23: mol_type = other RNA
attagcagaactacacaccaggg

SEQ ID NO: 212 - RNA, 23 nt, synthetic construct
  source 1..23: mol_type = other RNA
attggtcttaaaggtacctgagg

SEQ ID NO: 213 - RNA, 23 nt, synthetic construct
  source 1..23: mol_type = other RNA
tagcttgtagcaccatccaaagg

SEQ ID NO: 214 - RNA, 23 nt, synthetic construct
  source 1..23: mol_type = other RNA
gaactacacaccagggccagggg

SEQ ID NO: 215 - RNA, 23 nt, synthetic construct
  source 1..23: mol_type = other RNA
cagggaagtagccttgtgtgtgg

SEQ ID NO: 216 - RNA, 23 nt, synthetic construct
  source 1..23: mol_type = other RNA
tcagacccttttagtcagtgtgg

SEQ ID NO: 217 - RNA, 23 nt, synthetic construct
  source 1..23: mol_type = other RNA
ctgtggatctaccacacacaagg

SEQ ID NO: 218 - RNA, 23 nt, synthetic construct
  source 1..23: mol_type = other RNA
accagagtcacacaacagacggg

SEQ ID NO: 219 - RNA, 23 nt, synthetic construct
  source 1..23: mol_type = other RNA
gatatccactgacctttggatgg

SEQ ID NO: 220 - RNA, 23 nt, synthetic construct
  source 1..23: mol_type = other RNA
gacaagatatccttgatctgtgg

SEQ ID NO: 221 - RNA, 23 nt, synthetic construct
  source 1..23: mol_type = other RNA
agaactacacaccagggccaggg

SEQ ID NO: 222 - RNA, 23 nt, synthetic construct
  source 1..23: mol_type = other RNA
acactgactaaaagggtctgagg

SEQ ID NO: 223 - RNA, 23 nt, synthetic construct
  source 1..23: mol_type = other RNA
gggccacgtgatgaaatgctagg

SEQ ID NO: 224 - RNA, 23 nt, synthetic construct
  source 1..23: mol_type = other RNA
taaccagagagacccagtacagg

SEQ ID NO: 225 - RNA, 23 nt, synthetic construct
  source 1..23: mol_type = other RNA
tagcaccatccaaaggtcagtgg

SEQ ID NO: 226 - RNA, 23 nt, synthetic construct
  source 1..23: mol_type = other RNA
cactttttaaaagaaaagggggg

SEQ ID NO: 227 - RNA, 23 nt, synthetic construct
  source 1..23: mol_type = other RNA
tgagccagataagatagaagagg

SEQ ID NO: 228 - RNA, 23 nt, synthetic construct
  source 1..23: mol_type = other RNA
taccagagtcacacaacagacgg

SEQ ID NO: 229 - RNA, 23 nt, synthetic construct
  source 1..23: mol_type = other RNA
gattagcagaactacacaccagg

SEQ ID NO: 230 - RNA, 23 nt, synthetic construct
  source 1..23: mol_type = other RNA
agagagaagtgttagagtggagg

SEQ ID NO: 231 - RNA, 28 nt, synthetic construct
  source 1..28: mol_type = other RNA
ttccctgattagcagaactacacaccag

SEQ ID NO: 232 - RNA, 28 nt, synthetic construct
  source 1..28: mol_type = other RNA
ttctatcttatctggctcaactggtact

SEQ ID NO: 233 - RNA, 28 nt, synthetic construct
  source 1..28: mol_type = other RNA
ttctctcctttattggcctcttctatct

SEQ ID NO: 234 - RNA, 28 nt, synthetic construct
  source 1..28: mol_type = other RNA
ttctctctccgggtcatccatcccatgc

SEQ ID NO: 235 - RNA, 28 nt, synthetic construct
  source 1..28: mol_type = other RNA
ttcatcacgtggcccgagagctgcatcc

SEQ ID NO: 236 - RNA, 28 nt, synthetic construct
  source 1..28: mol_type = other RNA
ttcttgaagtactccggatgcagctctc

SEQ ID NO: 237 - RNA, 28 nt, synthetic construct
  source 1..28: mol_type = other RNA
ttcaagaactgctgacatcgagcttgct

SEQ ID NO: 238 - RNA, 28 nt, synthetic construct
  source 1..28: mol_type = other RNA
ttcttttaaaaagtggctaagatctaca

SEQ ID NO: 239 - RNA, 28 nt, synthetic construct
  source 1..28: mol_type = other RNA
ttccagtccccccttttcttttaaaaag

SEQ ID NO: 240 - RNA, 28 nt, synthetic construct
  source 1..28: mol_type = other RNA
ttcaagtagtgtgtgcccgtctgttgtg

SEQ ID NO: 241 - RNA, 28 nt, synthetic construct
  source 1..28: mol_type = other RNA
ttccacactgactaaaagggtctgaggg

SEQ ID NO: 242 - RNA, 28 nt, synthetic construct
  source 1..28: mol_type = other RNA
ttctttgggagtgaattagcccttccag

SEQ ID NO: 243 - RNA, 28 nt, synthetic construct
  source 1..28: mol_type = other RNA
ttcactcccaaagaagacaagatatcct

SEQ ID NO: 244 - RNA, 28 nt, synthetic construct
  source 1..28: mol_type = other RNA
ttctgctaatcagggaagtagccttgtg

SEQ ID NO: 245 - RNA, 28 nt, synthetic construct
  source 1..28: mol_type = other RNA
ttccgctggggactttccagggaggcgt

SEQ ID NO: 246 - RNA, 28 nt, synthetic construct
  source 1..28: mol_type = other RNA
ttccagggaggcgtggcctgggcgggac

SEQ ID NO: 247 - RNA, 28 nt, synthetic construct
  source 1..28: mol_type = other RNA
ttccctagttagccagagagctcccagg

SEQ ID NO: 248 - DNA, 60 bp, synthetic construct
  source 1..60: mol_type = other DNA
gggtctctctggttagaccagatctgagcctgggagctctctggctaactagggaaccca

SEQ ID NO: 249 - RNA, 23 nt, synthetic construct
  source 1..23: mol_type = other RNA
gttagaccagatctgagcctggg

SEQ ID NO: 250 - RNA, 23 nt, synthetic construct
  source 1..23: mol_type = other RNA
tagttagccagagagctcccagg

SEQ ID NO: 251 - RNA, 23 nt, synthetic construct
  source 1..23: mol_type = other RNA
gggagctctctggctaactaggg

SEQ ID NO: 252 - RNA, 23 nt, synthetic construct
  source 1..23: mol_type = other RNA
ggttagaccagatctgagcctgg

SEQ ID NO: 253 - RNA, 23 nt, synthetic construct
  source 1..23: mol_type = other RNA
agagctcccaggctcagatctgg

SEQ ID NO: 254 - RNA, 23 nt, synthetic construct
  source 1..23: mol_type = other RNA
atctgagcctgggagctctctgg

SEQ ID NO: 255 - RNA, 23 nt, synthetic construct
  source 1..23: mol_type = other RNA
tgggagctctctggctaactagg

SEQ ID NO: 256 - RNA, 28 nt, synthetic construct
  source 1..28: mol_type = other RNA
ttccctagttagccagagagctcccagg

(19) United States
(12) Patent Application Publication
(10) Pub. No.: US 2025/0387515 A1
(43) Pub. Date: Dec. 25, 2025

(54) HYPOIMMUNOGENIC BIOMIMETIC NANOVESICLE GENE EDITING SYSTEM FOR HIV INFECTION

(71) Applicant: Thomas MALCOLM, Andover, NJ (US)

(72) Inventor: Thomas MALCOLM, Andover, NJ (US)

(21) Appl. No.: 18/879,371

(22) PCT Filed: Jun. 28, 2023
(86) PCT No.: PCT/US2023/069287

§ 371 Date: Dec. 27, 2024

Related U.S. Application Data
(60) Provisional application No. 63/356,245, filed on Jun. 28, 2022.
(60) Provisional application No. 63/410,861, filed on Sep. 28, 2022.

(51) Int. Cl. A61K 48/00; C12N 9/22; C12N 15/11

(52) U.S. Cl. CPC A61K 48/0058; A61K 48/0041; C12N 9/226; C12N 15/11; C12N 2310/20

(57) ABSTRACT

Disclosed herein are compositions comprising allogeneic, hypoimmunogenic cell-targetable biomimetic nanovesicles (BioNVs) and methods of using the same for the treatment, prevention, and/or amelioration of HIV.

### FIELD

The present disclosure provides, in part, compositions and methods comprising allogeneic, hypoimmunogenic biomimetic nanovesicles and methods of using the same for the treatment or prevention of HIV infection, e.g., in a mammalian subject, such as a human.

### CROSS-REFERENCE TO RELATED APPLICATIONS

This application claims the benefit of and priority to U.S. Provisional Application No. 63/356,245, filed Jun. 28, 2022, and U.S. Provisional Application No. 63/410,861, filed Sep. 28, 2022, the contents of which are herein incorporated by reference in their entirety.

### DESCRIPTION OF THE TEXT FILE SUBMITTED ELECTRONICALLY

The contents of the text file submitted electronically herewith are incorporated herein by reference in their entirety: a computer-readable XML format copy of the Sequence Listing (filename: “CAR-007PC_Sequence_Listing.xml”; date recorded Jun. 27, 2023; file size: 227,743 bytes).

### BACKGROUND

In people living with viral infection such as Human Immunodeficiency Virus (HIV) (PLWH) on antiretroviral therapy (ART), the virus persists in a latent form where there is minimal transcription or protein expression. Latently infected cells are a major barrier to curing HIV, among curing other viruses with latency phases.

Current methods for the elimination of the HIV genome focus on removing the fully translated genomic region of the integrated virus, however this leads to very limited success in the elimination of latent virus due to several impassable challenges. For example, non-elimination issues: the number of latently infected cells must be reduced to levels that require saturating conditions by the gene editor and the gene editor delivery system. The number of cells required for the re-establishment of infection and the resultant latent pools is ultra-low (e.g., on the order of 10¹-10² cells). Therefore, current therapeutic delivery approaches are inadequate to effectively reach all infected cells and are potentially mathematically limited to an extremely small group of HIV controller (HIC) patients that are functionally cured of HIV-1, with no long-term efficacious value to the broader patient population.

The use of AAV9 (among other AAVs) as delivery systems for CRISPR editors introduce packaging size constraints that limit the editor to smaller and fewer endonucleases. This is generally associated with: i) off-target effects and indel introduction, ii) severely delimited number of targeting gRNAs which is inadequate to address multi-mutational sequences that are associated with HIV-1 strains, iii) are predisposed to existing immune reactivity in the majority of patients (therefore not usable), iv) are limited in redosing (therefore patients that rebound are untreatable with the same or identical vectors), v) are costly to manufacture at scales that make off-the-shelf treatment possible, and vi) represent only a temporary treatment that halts the virus in an approach synonymous to ART.

Newer approaches such a LNPs address some shortcomings of AAVs for the delivery of CRISPR gene editors with larger payloads and selective tropism but still exhibit several disadvantages, such as: i) inefficient tissue penetration, ii) difficulties engineering barrier-crossing properties, iii) tissue tropism issues, iv) subject to potential opsonization, v) can cause inflammation and direct or indirect cellular damage, vi) can cause a release of exosomes from targeted cells leading to inflammation, and vii) liver and kidney sinking (Herskovitz, et al. “CRISPR-Cas9 Mediated Exonic Disruption for HIV-1 Elimination.” EBioMedicine Vol. 73, 2021: 103678), to name a few. Although the LNP-based CRISPR approaches have advantages from a delivery perspective, they fail to consider the high mutation rates of HIV across all clades (Herskovitz et al.).

Exosomes have yet to be applied in practice as a delivery vehicle for CRISPR gene editing endonucleases to treat HIV. Exosome systems have several caveats for use in treating HIV, such as: i) engineering of targeting receptors in a consistent manner, ii) constrained by manufacturing challenges, iii) limited to cytoplasmic membrane transmembrane receptor ligand densities from which they are derived, iv) standardization and consistency among batch protocols (high heterogeneity), and v) poor cell and tissue penetration properties compared to other types of cell-derived vesides (CDVs).

Although there are infection management options for HIV, there is presently a paucity of effective curative treatments for HIV that also eliminate latently infected cells. Several caveats exist in developing treatment options which can fully eliminate latently infected cells, for example, targeting of therapeutics to diseased cells lacking viral antigens. Cell-based therapies, such as chimeric antigen receptor engineered T cells (CAR-T cells) are currently in development for treatment of many infectious diseases, owning to the enhanced targeting of specific cell subsets and the ability to delivery cytotoxic payloads, gene editing payloads, etc. However, in developing cell therapies, there are inherent obstacles in the way of immune incompatibilities, immune and cellular exhaustion, poor cell-directed penetrance, manufacturing costs, autologous cell sources, regulation of cytokine production, to name a few.

Therefore, there remains a need for therapies that are useful for treating HIV (among other chronic diseases caused by viruses) that take advantage of packaging and targeting advantages of cell-based therapies while overcoming their shortcomings.

### SUMMARY

Accordingly, in aspects, the present invention relates to a biomimetic nanovesicle (BioNV) for treating HIV (and with some adjustments, other retroviruses or latent viruses), wherein the BioNV comprises a targeting agent that is targeted against at least a first cell surface marker of a latently HIV-infected cell, and wherein the BioNV is configured to deliver a gene editing payload comprising at least two guide RNAs (gRNAs).

In aspects, the present invention relates to a biomimetic nanovesicle (BioNV) for treating HIV, wherein the BioNV comprises a targeting agent that is targeted against at least a first cell surface marker of a latently HIV-infected cell, and wherein the BioNV is configured to deliver: a) a gene editing payload comprising at least two gRNAs, and b) a small RNA.

In embodiments, the BioNV is derived from a hypoimmunogenic modified cell. In embodiments, the hypoimmunogenic modified cell is a stem cell, an induced pluripotent stem cell (iPSC), a reprogrammed pluripotent or multipotent cell, an embryonic stem cell, a mesenchymal stem cell, or a differentiated cell which originates from any modified cell thereof. In embodiments, the hypoimmunogenic modified cell is an iPSC. In embodiments, the hypoimmunogenic modified cell is a T cell, helper T cell, T-memory cell, γδ T cell, NK cell, monocyte, or macrophage.

In embodiments, the BioNV substantially lacks one or more MHC class I proteins, MHC class II proteins, T cell receptor (TCR) proteins, and/or cytokine release syndrome (CRS) proteins. In embodiments, the BioNV has reduced or ablated β2-macroglobulin (B2M) protein expression and/or activity and/or MHC class I protein expression and/or activity. In embodiments, the BioNV has reduced or ablated CIITA protein expression and/or activity and/or MHC class II protein expression and/or activity.

In embodiments, the BioNV has reduced or ablated human leukocyte antigen A (HLA-A) protein expression and/or activity. In embodiments, the BioNV has reduced or ablated human leukocyte antigen B (HLA-B) protein expression and/or activity. In embodiments, the BioNV has reduced or ablated human leukocyte antigen C (HLA-C) protein expression and/or activity. In embodiments, the BioNV has reduced or ablated human leukocyte antigen E (HLA-E) or human leukocyte antigen G (HLA-G) protein expression and/or activity. In embodiments, the BioNV has reduced or ablated human leukocyte antigen F (HLA-F) protein expression and/or activity.

In embodiments, the BioNV has reduced or ablated T cell alpha constant (TRAC) protein expression and/or activity. In embodiments, the BioNV has reduced or ablated T cell beta constant (TRBC) protein expression and/or activity. In embodiments, the BioNV has reduced or ablated programmed cell death 1 protein (PD-1) expression and/or activity; or wherein the BioNV has PD-1 protein expression and/or activity. In embodiments, the BioNV has reduced or ablated interleukin-4 (IL-4) protein expression and/or activity. In embodiments, the BioNV has reduced or ablated interleukin-6 (IL-6) protein expression and/or activity. In embodiments, the BioNV has reduced or ablated interleukin-10 (IL-10) protein expression and/or activity. In embodiments, the BioNV has reduced or ablated interleukin-16 (IL-16) protein expression and/or activity.

In embodiments, the BioNV has SerpinB9 protein expression and/or activity. In embodiments, the BioNV has CD34 protein expression and/or activity. In embodiments, the BioNV has CCL2 protein expression and/or activity. In embodiments, the BioNV has PD-L1 protein expression and/or activity. In embodiments, wherein the BioNV has H2-M3 protein expression and/or activity. In embodiments, the BioNV has CD47 protein expression and/or activity. In embodiments, the BioNV has CD24 protein expression and/or activity. In embodiments, the BioNV has chimeric CD24/CD47 protein expression and/or activity. In embodiments, the BioNV has CD200 protein expression and/or activity. In embodiments, the BioNV has chimeric CD24/CD200 protein expression and/or activity or chimeric CD47/CD200 protein and/or activity. In embodiments, the BioNV has CTLA-4 protein expression and/or activity. In embodiments, the BioNV has MFG-E8 protein expression and/or activity. In embodiments, the BioNV has NCAM protein expression and/or activity. In embodiments, the BioNV has a-phagocytic integrin protein expression and/or activity. In embodiments, the BioNV has FasL protein expression and/or activity.

In embodiments, the BioNV has reduced or ablated protein expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins. In embodiments, the BioNV has expression and/or activity of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, the BioNV is allogeneic. In embodiments, the BioNV does not cause an adverse immune reaction in subjects to which it is administered.

In embodiments, the BioNV substantially lacks protein and/or activity of HLA-A, HLA-B, HLA-C, HLA-F, CIITA, IL-6, TRAC, TRBC, and one of either HLA-E or HLA-G.

In embodiments, the BioNV substantially lacks protein and/or activity of HLA-A, HLA-B, HLA-C, HLA-F, CIITA, IL-6, TRAC, TRBC, PD-1, and one of either HLA-E or HLA-G.

In embodiments, the BioNV substantially lacks protein and/or activity of HLA-A, HLA-B, HLA-C, HLA-F, CIITA, IL-6, TRAC, TRBC, PD-1, one of either HLA-E or HLA-G, and one or more of IL4, IL-10, and IL-16.

In embodiments, the BioNV has membrane-embedded a-phagocytic integrin, CCL2, H2-M3, FasL, MFEG8, and PD-L1 and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV has membrane-embedded a-phagocytic integrin, CCL2, H2-M3, FasL, MFEG8, SerpinB9, and PD-L1 and/or CTLA-4, and one of either CD24, CD47, CD200, chimeric CD24/CD47, chimeric CD24/CD200, and chimeric CD47/CD200, or two of either CD24, CD47, and CD200.

In embodiments, the BioNV has membrane-embedded CD200 protein and substantially lacks protein of either CD24 or CD47.

In embodiments, the targeting agent is one or more of a CAR, VERR, viral ligand, viral receptor, or an antibody or antibody format selected from a monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody or antibody format. In embodiments, the VERR or viral ligand is a gp120/gp41 complex. In embodiments, the targeting agent is a scFv.

In embodiments, the targeting agent is a CAR. In embodiments, the CAR comprises a transmembrane domain of or derived from CD28, CD3ζ, CD4, CD8α, ICOS, or fragment and/or combination thereof. In embodiments, the CAR comprises an intracellular domain further comprising an intracellular signaling domain of a CD3ζ chain and/or one or more co-stimulatory molecules, optionally selected from CD28, 4-1BB, ICOS, CD27, and OX40. In embodiments, the CAR is activated, optionally wherein the CAR is activated via its target, through another receptor, and/or a virus.

In embodiments, the first cell surface marker is or comprises a sialic acid-binding immunoglobulin-like lectin (Siglec) molecule. In embodiments, the Siglec molecule is Siglec-1. In embodiments, the first cell surface marker is or comprises CD2, CD3, CD4, CCR5, CD20, CD25, CD30, CD32a, CD69, CD91, CD160, CD257, LAG-3, CD147, CD231, CEACAM1, PLXNB2, HLA-DR, PD-1, CTLA-4, TIGIT, LAG-3, TIM-3, BTK, Cyclophilin B, Sec62, Rab10, or SPCS.

In embodiments, the gene editing payload comprises one or more gene editors. In embodiments, the one or more gene editors forms a complex with at least one of the two gRNAs. In embodiments, the one or more gene editors is a site-directed endonuclease, CRISPR/Cas nuclease, SpCas9-HF1, Cpf1, CasX, C201, C2c2, C2c3, Cas9, TevCas9, Archaea Cas9, CasY.1, CasY.2, CasY.3, CasY.4, CasY.5, CasY.6, Cas omega, transposase, and/or an ortholog or homolog thereof. In embodiments, the gene editor is a CRISPR/Cas nuclease. In embodiments, the gene editor is a site-directed endonuclease.

In embodiments, the one or more gene editors excises the HIV proviral genomic sequence between the at least two gRNA target sequences in the latently HIV-infected cell. In embodiments, the excision retains in the latently HIV-infected cell an integrated 5′ LTR sequence, TAR Loop sequence, and an at least a portion of a Gag coding sequence. In embodiments, the excision retains in the latently HIV-infected cell an integrated 3′ LTR sequence and at least a portion of a Nef coding sequence.

In embodiments, the excision retains in the latently HIV-infected cell the integrated 5′ LTR sequence, TAR Loop sequence, at least a portion of the Gag coding sequence, at least a portion of the Nef coding sequence, the 3′ LTR sequence, and at least one stop codon downstream of the Gag coding sequence and upstream of the Nef coding sequence and at least one start codon downstream of the stop codon and upstream of the Nef coding sequence.

In embodiments, the excision retains in the latently HIV-infected cell the integrated 5′ LTR sequence, TAR Loop sequence, at least a portion of the Gag coding sequence, and 3′ LTR sequence.

In embodiments, the excision disrupts an integrated HIV TAR loop sequence. In embodiments, the excision retains in the latently HIV-infected cell at least a portion of an integrated 5′ LTR, at least a portion of an HIV Env sequence, and at least a portion of an HIV Nef sequence.

In embodiments, at least two gRNAs comprise a first gRNA complementary to a first coding sequence and/or a first non-coding sequence, and a second gRNA complementary to a second coding sequence and/or a second non-coding sequence.

In embodiments, the first non-coding sequence is an integrated HIV TAR loop sequence. In embodiments, the first non-coding sequence is 5′ of an integrated HIV TAR loop sequence and/or within a 5′ LTR sequence.

In embodiments, the first coding sequence is an HIV Gag proviral genomic sequence. In embodiments, an excision site corresponding to the first coding sequence is at least about 21 base pairs (bp) downstream of the Gag protein start codon.

In embodiments, the second coding sequence and/or the second non-coding sequence is downstream of the first coding sequence and/or the first non-coding sequence and upstream of at least portion of an integrated HIV Env sequence.

In embodiments, the second coding sequence is an HIV Nef proviral genomic sequence, or wherein the second non-coding sequence is an HIV 3′ LTR genomic sequence. In embodiments, an excision site corresponding to the second coding sequence is at least about 21 base pairs (bp) upstream of the Nef protein stop codon.

In embodiments, the at least two gRNAs comprise; one or more nucleic sequences selected from SEQ ID NOs: 1-20, or one or more nucleic acids comprising one or more substitutions, insertions, or deletions thereof, that is optionally compatible with Cas9 and optionally targets Nef, one or more nucleic sequences selected from SEQ ID NOs: 21-31, or one or more nucleic acids comprising one or more substitutions, insertions, or deletions thereof, that is optionally compatible with Cpf1/CasX and optionally targets Nef, one or more nucleic sequences selected from SEQ ID NOs: 32-51, or one or more nucleic acids comprising one or more substitutions, insertions, or deletions thereof, that is optionally compatible with Cas9 and optionally targets Pol, one or more nucleic sequences selected from SEQ ID NOs: 52-71, or one or more nucleic acids comprising one or more substitutions, insertions, or deletions thereof, that is optionally compatible with Cpf1/CasC and optionally targets Pol, one or more nucleic sequences selected from SEQ ID NOs: 72-91, or one or more nucleic acids comprising one or more substitutions, insertions, or deletions thereof, that is optionally compatible with Cas9 and optionally targets Rev, one or more nucleic sequences selected from SEQ ID NOs: 92-107, or one or more nucleic acids comprising one or more substitutions, insertions, or deletions thereof, that is optionally compatible with Cpf1/CasX and optionally targets Rev, one or more nucleic sequences selected from SEQ ID NOs: 108-127, or one or more nucleic acids comprising one or more substitutions, insertions, or deletions thereof, that is optionally compatible with Cas9 and optionally targets Gag, one or more nucleic sequences selected from SEQ ID NOs: 128-147, or one or more nucleic acids comprising one or more substitutions, insertions, or deletions thereof, that is optionally compatible with Cpf1/CasX and optionally targets Gag, one or more nucleic sequences selected from SEQ ID NOs: 148-166, or one or more nucleic acids comprising one or more substitutions, insertions, or deletions thereof, that is optionally compatible with Cas9 and optionally targets Tat, one or more nucleic sequences selected from SEQ ID NOs: 167-193, or one or more nucleic acids comprising one or more substitutions, insertions, or deletions thereof, that is optionally compatible with Cas9 and optionally targets a 5′LTR sequence of HXB2, one or more nucleic sequences selected from SEQ ID NOs: 194-210, or one or more nucleic acids comprising one or more substitutions, insertions, or deletions thereof, that is optionally compatible with Cpf1/CasX and optionally targets a 5′LTR sequence of HXB2, one or more nucleic sequences selected from SEQ ID NOs: 211-230, or one or more nucleic acids comprising one or more substitutions, insertions, or deletions thereof, that is optionally compatible with Cas9 and optionally targets a 3′LTR sequence of HXB2, one or more nucleic sequences selected from SEQ ID NOs: 231-247, or one or more nucleic acids comprising one or more substitutions, insertions, or deletions thereof, that is optionally compatible with Cpf1/CasX and optionally targets a 3′LTR sequence of HXB2, one or more nucleic sequences selected from SEQ ID NOs: 249-254, or one or more nucleic acids comprising one or more substitutions, insertions, or deletions thereof, that is optionally compatible with Cas9 and optionally targets HXB2 TAR, and/or a nucleic sequence of SEQ ID NOs: 255, or a nucleic acid comprising one or more substitutions, insertions, or deletions thereof, that is optionally compatible with Cpf1/CasX and optionally targets HXB2 TAR.

In embodiments, the small RNA targets an HIV coding sequence. In embodiments, the small RNA targets an HIV Gag coding sequence or an HIV Nef coding sequence. In embodiments, the HIV Nef coding sequence is a portion of a messenger RNA encoding a Nef protein.

In embodiments, the small RNA is configured to interact with an RNA-induced silencing complex (RISC) reservoirs in the latently HIV-infected cell. In embodiments, the interaction with the RISC complex induces silencing of the HIV Nef RNA sequence. In embodiments, the interaction with the RISC complex induces amplification of the small RNA. In embodiments, the small RNA is configured to be trafficked from the latently HIV-infected cell to at least a second HIV-infected cell. In embodiments, the small RNA functions to prevent and/or reverse Nef-mediated multiple histocompatibility complex (MHC) sequestration in the latently HIV-infected cell and/or the second HIV-infected cell. In embodiments, the trafficking is through a SIDT-1/2 cell surface protein complex.

In embodiments, the small RNA is one or more of a tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense digonucleotide (ASO), locked nucleic acid (LNA), splice switching oligonucleotide (SSO), tRNA, complementary messenger RNA, repeat associated small interfering RNA (rasiRNA), and small non-coding RNA. In embodiments, the small RNA is a RNAi. In embodiments, the small RNA is a shRNA.

In embodiments, the BioNV encapsulates one or more additional nucleic acids. In embodiments, the one or more additional nucleic acids encodes one or more gene editing payloads. In embodiments, the one or more gene editing payloads is a CRISPR endonuclease and/or site-directed endonuclease. In embodiments, the one or more additional nucleic acids encodes one or more gRNA sequences. In embodiments, the one or more additional nucleic acids encodes one or more small RNAs that targets an HIV sequence. In embodiments, the one or more additional nucleic acids encodes an HIV Tat sequence. In embodiments, the one or more additional nucleic acids encodes one or more aptamers configured to bind an HIV TAR sequence. In embodiments, the one or more additional nucleic acids comprises one or more low-level expression constitutively active promoters, tissue-specific promoters, cell-specific promoters, and/or suicide promoters.

In embodiments, the BioNV encapsulates one or more latency reversal agents (LRAs). In embodiments, the BioNV encapsulates one or more antiretroviral therapies. In embodiments, the antiretroviral therapy is a non-nucleoside reverse transcriptase inhibitor (NNRTI), optionally selected from efavirenz (SUSTIVA), rilpivirine (EDURANT) and doravirine (PIFELTRO). In embodiments, the antiretroviral therapy is a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY). In embodiments, the antiretroviral therapy is a protease inhibitor (PI), optionally selected from atazanavir (REYATAZ), darunavir (PREZISTA), and lopinavir/ritonavir (KALETRA). In embodiments, the antiretroviral therapy is an integrase inhibitor, optionally selected from bictegravir sodium/emtricitabine/tenofovir alafenamide fumar (BIKTARVY), raltegravir (ISENTRESS), and dolutegravir (TIVICAY). In embodiments, the antiretroviral therapy is an entry or fusion inhibitor, optionally selected from enfuvirtide (FUZEON) and maraviroc (SELZENTRY).

In embodiments, the BioNV is about 10 nm to about 1200 nm in size. In embodiments, the BioNV is about 10 nm to about 100 nm in size. In embodiments, the BioNV is about 100 nm to about 200 nm in size. In embodiments, the BioNV is about 200 nm to about 500 nm in size. In embodiments, the BioNV is about 500 nm to about 1200 in size.

In embodiments, the BioNV is stored at about −80° C. or suitable for storage at about −80° C. In embodiments, the BioNV is lyophilized or suitable for lyophilization.

In aspects, the present invention relates to methods of treating an HIV infection comprising administering to a subject in need thereof a BioNV, as described herein.

In embodiments, the HIV is HIV-1 or HIV-2. In embodiments, the HIV-1 is one of Group M, Group N, Group O, and Group P. In embodiments, the HIV-1 is Group M and a subtype selected from subtype A, B, C, D, F, G, H, I, J, K, and L.

In embodiments, administering the BioNV results in excision by the gene editing payload of the HIV proviral genome between the target sequences of the at least two gRNAs.

In embodiments, the excision by the one or more gene editors retains in the latently HIV-infected cell an integrated 5′ LTR sequence, TAR Loop sequence, and an at least a portion of a Gag coding sequence. In embodiments, the excision by the one or more gene editors retains in the latently HIV-infected cell an integrated 3′ LTR sequence and at least a portion of a Nef coding sequence.

In embodiments, the excision by the one or more gene editors retains the integrated 5′ LTR sequence, TAR Loop sequence, at least a portion of the Gag coding sequence, at least a portion of the Nef coding sequence, the 3′ LTR sequence, and at least one stop codon downstream the Gag coding sequence and upstream the Nef coding sequence and at least one start codon downstream the stop codon and upstream the Nef coding sequence.

In embodiments, the excision by the one or more gene editors retains the integrated 5′ LTR sequence, TAR Loop sequence, at least a portion of the Gag coding sequence, and 3′ LTR sequence.

In embodiments, the excision disrupts an integrated HIV TAR loop sequence. In embodiments, the excision retains in the latently HIV-infected cell at least a portion of an integrated 5′ LTR sequence, at least a portion of an HIV Env sequence, and at least a portion of an HIV Nef sequence.

In embodiments, administering the BioNV results in the expression of one or more HIV proteins or one or more portions thereof in an HIV-infected cell. In embodiments, the one or more HIV proteins is p1, p2, p6, p7, p17, p24, gp41, gp120, or an HIV polymerase. In embodiments, the one or more HIV proteins is processed and presented via a multiple histocompatibility complex (MHC) in the latently HIV-infected cell.

In embodiments, the BioNV delivers a small RNA. In embodiments, administering the BioNV results in expression of a partial Nef RNA (pNef). In embodiments, the pNef is targeted by the small RNA. In embodiments, the small RNA functions to prevent and/or reverse Nef-mediated MHC sequestration in the latently HIV-infected cell. In embodiments, the small RNA interacts with an RNA-induced silencing complex (RISC) in the latently HIV-infected cell. In embodiments, the interaction between the small RNA and the RISC in the latently HIV-infected cell results in amplification of the small RNA.

In embodiments, the small RNA is configured to be trafficked from the latently HIV-infected cell to at least a second HIV-infected cell. In embodiments, the trafficking is through a SIDT-1/2 cell surface protein complex in the latently HIV-infected cell.

In embodiments, administering the BioNV results in γδ T cell costimulation of NK cells in the subject. In embodiments, the BioNV results in restoration of the γδ T cell population in the subject in relation to the subject prior to the administration, and/or in relation to a subject not administered the BioNV. In embodiments, the γδ T cell is of a Vγ9JP repertoire. In embodiments, administering the BioNV results in T cell-mediated cytotoxicity against one or more HIV-infected cells.

In embodiments, administering the BioNV results in expression of a Tat sequence. In embodiments, administering the BioNV results in aptamer binding of a TAR sequence. In embodiments, administering the BioNV results in delivery of one or more latency reversal agents (LRAs).

In embodiments, the BioNV encapsulates one or more antiretroviral therapies. In embodiments, the methods herein further comprise administering one or more antiretroviral therapies.

In embodiments, the antiretroviral therapy is a non-nucleoside reverse transcriptase inhibitor (NNRTI), optionally selected from efavirenz (SUSTIVA), rilpivirine (EDURANT) and doravirine (PIFELTRO). In embodiments, the antiretroviral therapy is a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY). In embodiments, the antiretroviral therapy is a protease inhibitor (PI), optionally selected from atazanavir (REYATAZ), darunavir (PREZISTA), and lopinavir/ritonavir (KALETRA). In embodiments, the antiretroviral therapy is an integrase inhibitor, optionally selected from bictegravir sodium/emtricitabine/tenofovir alafenamide fumar (BIKTARVY), raltegravir (ISENTRESS), and dolutegravir (TIVICAY). In embodiments, the antiretroviral therapy is an entry or fusion inhibitor, optionally selected from enfuvirtide (FUZEON) and maraviroc (SELZENTRY).

In embodiments, the antiretroviral therapy comprises at least two, or at least three antiretroviral therapies, or at least four of the antiretroviral therapies in combination. In embodiments, administering comprises providing one or more doses of at least about 1 ng/kg to at least about 10 mg/kg of BioNVs. In embodiments, the administration is intravenous, intramuscular, or parenteral.

In aspects, the present invention relates to methods of treating an HIV infection in a subject comprising administering to the subject a biomimetic nanovesicle (BioNV), wherein the BioNV comprises a targeting moiety that is targeted against at least a first cell surface marker of a latently HIV-infected cell, and wherein the BioNV is configured to deliver: a) a gene editing payload comprising at least two gRNAs, wherein the at least two gRNAs comprise: i) a first gRNA targeted against an HIV Gag proviral genomic sequence, and ii) a second gRNA targeted against an HIV proviral 3′ LTR genomic sequence, wherein the gene editing payload is capable of excising the HIV proviral genomic sequence between the HIV Gag proviral genomic sequence and the HIV 3′ LTR proviral genomic sequence, and wherein an HIV 5′ LTR sequence, TAR Loop sequence, at least a portion of the HIV Gag proviral genomic sequence, and at least a portion of the HIV 3′ LTR proviral genomic sequence is not excised from the latently HIV-infected cell.

In aspects, the present invention relates to methods of treating an HIV infection in a subject comprising administering to the subject a biomimetic nanovesicle (BioNV), wherein the BioNV comprises a targeting moiety that is targeted against at least a first cell surface marker of a latently HIV-infected cell, and wherein the BioNV is configured to deliver: a) a gene editing payload comprising at least two gRNAs, wherein the at least two gRNAs comprise: i) a first gRNA targeted against an HIV Gag proviral genomic sequence, and ii) a second gRNA targeted against an HIV proviral Nef genomic sequence, wherein the gene editing payload is capable of excising the HIV proviral genomic sequence between the HIV Gag proviral genomic sequence and the HIV Nef proviral genomic sequence, and wherein an HIV 5′ LTR sequence, TAR Loop sequence, at least a portion of the HIV Gag proviral genomic sequence, and at least a portion of the HIV Nef proviral genomic sequence is not excised from the latently HIV-infected cell; and b) an interference RNA (RNAi), wherein the RNAi is targeted against a messenger RNA sequence complementary to the portion of the HIV Nef proviral genomic sequence that is not excised.

In aspects, the present invention relates to methods of treating an HIV infection in a subject comprising administering to the subject a biomimetic nanovesicle (BioNV), wherein the BioNV comprises a targeting moiety that is targeted against at least a first cell surface marker of a latently HIV-infected cell, and wherein the BioNV is configured to deliver: a) a gene editing payload comprising at least two gRNAs, wherein the at least two gRNAs comprise: i) a first gRNA targeted against an HIV Gag proviral genomic sequence, and ii) a second gRNA targeted against an HIV Nef proviral genomic sequence, wherein the gene editing payload is capable of excising the HIV proviral genomic sequence between the HIV Gag proviral genomic sequence and the HIV Nef proviral genomic sequence, and wherein at least a portion of a 5′ end of the HIV proviral genomic sequence and a portion of a 3′ end of the HIV proviral genomic sequence is not excised from the latently HIV-infected cell; and b) an interference RNA (RNAi), wherein the RNAi is targeted against a messenger RNA sequence complementary to the portion of the HIV Nef proviral genomic sequence that is not excised, wherein the latently HIV-infected cell expresses a partial Nef RNA sequence (pNef) from the portion of the HIV Nef proviral genomic sequence which reduces Nef-mediated MHC complex sequestration in the latently HIV-infected cell, wherein the latently HIV-infected cell expresses a partial Gag protein (pGag) from the portion of the portion of the HIV Gag proviral genomic sequence which is recognized by the MHC complex and results in T-cell stimulation against the latently HIV-infected cell, and wherein the pNef and pGag expression in the latently HIV-infected cell results in: a) processing and presentation of the pGag via an MHC, b) cell-to-cell spread of the RNAi via the SIDT-1/2 complex, and c) T-cell mediated cytotoxicity against one or more latently HIV-infected cells.

In aspects, the present invention relates to methods of treating an HIV infection in a subject comprising administering to the subject a biomimetic nanovesicle (BioNV), wherein the BioNV comprises a targeting moiety that is targeted against at least a first cell surface marker of a latently HIV-infected cell, and wherein the BioNV is configured to deliver: a) a gene editing payload comprising at least two gRNAs, wherein the at least two gRNAs comprise: i) a first gRNA targeted against an HIV proviral genomic sequence within the TAR loop, 5′ of the TAR loop, e.g. immediately 5′ of the TAR loop; and ii) a second gRNA targeted against upstream of at least a portion of an HIV Env proviral genomic sequence, wherein the gene editing payload is capable of excising the HIV proviral genomic sequence between the first gRNA target sequence and the second gRNA target sequence, and wherein at least a portion of a 5′ end of the HIV proviral genomic sequence and a portion of the HIV Env proviral genomic sequence is not excised from the latently HIV-infected cell; and b) an interference RNA (RNAi), wherein the RNAi is targeted against a messenger RNA sequence complementary to a portion of an HIV Nef proviral genomic sequence that is not excised, wherein the latently HIV-infected cell expresses a partial Nef RNA sequence (pNef) from the portion of the HIV Nef proviral genomic sequence which reduces Nef-mediated MHC complex sequestration in the latently HIV-infected cell, wherein the latently HIV-infected cell expresses a partial gp120 and/or gp41 peptide from the portion of the HIV Env proviral genomic sequence that is not excised, which is recognized by the MHC complex and results in T-cell stimulation against the latently HIV-infected cell, and wherein the pNef and gp120/gp41 expression in the latently HIV-infected cell results in: processing and presentation of gp120/gp41 peptides via an MHC; cell-to-cell spread of the RNAi via the SIDT-1/2 complex; and T-cell mediated cytotoxicity against one or more latently HIV-infected cells.

In aspects, the present invention relates to methods of treating an HIV infection in a subject comprising administering to the subject a biomimetic nanovesicle (BioNV), wherein the BioNV comprises a targeting moiety that is targeted against at least a first cell surface marker of a latently HIV-infected cell, and wherein the BioNV is configured to deliver: a) a gene editing payload comprising at least two gRNAs, wherein the at least two gRNAs comprise: i) a first gRNA targeted against an HIV Gag proviral genomic sequence; and ii) a second gRNA targeted against upstream of at least a portion of an HIV Env proviral genomic sequence, wherein the gene editing payload is capable of excising the HIV proviral genomic sequence between the first gRNA target sequence and the second gRNA target sequence, and wherein at least a portion of an HIV TAR loop and 5′ LTR proviral genomic sequence and at least a portion of the HIV Env proviral genomic sequence is not excised from the latently HIV-infected cell; and b) an interference RNA (RNAi), wherein the RNAi is targeted against a messenger RNA sequence complementary to a portion of an HIV Nef proviral genomic sequence that is not excised, wherein the latently HIV-infected cell expresses a partial Nef RNA sequence (pNef) from the portion of the HIV Nef proviral genomic sequence which reduces Nef-mediated MHC complex sequestration in the latently HIV-infected cell, wherein the latently HIV-infected cell expresses a partial gp120 and/or gp41 peptide from the portion of the HIV Env proviral genomic sequence that is not excised, which is recognized by the MHC complex and results in T-cell stimulation against the latently HIV-infected cell, and wherein the pNef and gp120/gp41 expression in the latently HIV-infected cell results in: a) processing and presentation of gp120/gp41 peptides via an MHC; b) cell-to-cell spread of the RNAi via the SIDT-1/2 complex; and c) T-cell mediated cytotoxicity against one or more latently HIV-infected cells.

### BRIEF DESCRIPTION OF THE DRAWINGS

FIGS. 1A and 1B depict non-limiting diagrammatic representations illustrating an exemplary HIV-1 proviral genome gene editing strategy for T cell stimulation with an interference RNA approach. The HIV-1 integrated proviral genome is ˜9200 bp in length, flanked by long terminal repeats (LTRs). A BioNV-delivered gene editing payload and gRNAs target Gag (gRNA CRV1) and Nef (gRNA CRV2), resulting in excision of the spanning sequence between the gRNA targets (FIG. 1A). The retained genomic construct results in expression of a partial Gag (pGag) protein (p17 and/or p24) and a partial Nef mRNA transcript (pNef) (FIG. 1A). A BioNV can deliver a plasmid (e.g., containing the mechanistic properties to direct and pass it into the nucleus in a cell) encoding one or more gene editing payloads (e.g., a CRISPR endonuclease), gRNA(s), shRNA, a Tat expression cassette, and/or a TAR loop binding aptamer (FIG. 1B). Expression of pGag and/or pNef can be driven by the TAR loop and initiator sequence (Inr) via Tat expression from a promoter on the same plasmid as the gene editing payload and/or by expression of an activating TAR loop-binding aptamer (FIG. 1B).

FIGS. 2A and 2B depict non-limiting diagrammatic representations illustrating an exemplary HIV-1 proviral genome gene editing strategy for T cell stimulation without an interference RNA approach. The HIV-1 integrated proviral genome is ˜9200 bp in length, flanked by long terminal repeats (LTRs). A BioNV-delivered gene editing payload and gRNAs target Gag (gRNA CRV Gag Pol) and 3′ LTR (gRNA CRV 3′UTR), resulting in excision of the spanning sequence between the gRNA targets (FIG. 2A). The retained genomic construct results in expression of a partial Gag (pGag) protein (p1, p2, p6, p7, p17, and/or p24) and/or partial polymerase peptides (FIG. 2A). A BioNV can deliver a plasmid (e.g., containing the mechanistic properties to direct and pass it into the nucleus in a cell) encoding one or more gene editing payloads (e.g., a CRISPR endonuclease), gRNA(s), a Tat expression cassette, and/or a TAR loop binding aptamer (FIG. 2B). Expression of pGag can be driven by the TAR loop and initiator sequence (Inr) via Tat expression from a promoter on the same plasmid as the gene editing payload and/or by expression of an activating TAR loop-binding aptamer (FIG. 2B).

FIG. 3 depicts a non-limiting diagrammatic representation illustrating an exemplary HIV-1 proviral genome gene editing strategy for T cell stimulation with an interference RNA approach. The HIV-1 integrated proviral genome is ˜9200 bp in length, flanked by long terminal repeats (LTRs). A BioNV-delivered gene editing payload and gRNAs target the 5′ end of the HIV proviral genome (gRNA CRV1) and 5′ end of the Env proviral sequence (gRNA CRV2), resulting in excision of the spanning sequence between the gRNA targets. The gRNA CRV1 can disrupt the TAR loop and Inr sequence by guiding endonuclease activity within the sequence, or immediately 5′ of the sequence for excision. Alternatively, the gRNA CRV1 can guide endonuclease activity downstream of the TAR loop region so the cell can retain the sequence. The retained genomic construct results in expression of at least a portion of Env (gp120/gp41 protein) and Nef. A BioNV can deliver a plasmid (e.g., containing the mechanistic properties to direct and pass it into the nucleus in a cell) encoding one or more gene editing payloads (e.g., a CRISPR endonuclease), gRNA(s), shRNA, a Tat expression cassette, and/or a TAR loop binding aptamer. Expression of Env and Nef can be driven by the TAR loop and initiator sequence (Inr) via Tat expression from a promoter on the same plasmid as the gene editing payload and/or by expression of an activating TAR loop-binding aptamer. Alternatively, low levels of background expression can occur without the 5′ TAR loop and Inr sequences.

FIG. 4 depicts a non-limiting diagrammatic representation of T cell populations after HIV infection.

FIGS. 5A and 58 depict non-limiting diagrammatic representations illustrating an exemplary T cell stimulation strategy for CRISPR/Cas-mediated HIV genome excision and partial disruption of HIV Nef leading to the inhibition of MHC suppression and HIV peptide presentation for T cell-mediated response (FIG. 5A). MHC-to-TLR mediated activation initiates a bidirectional signaling cascade (FIG. 58). T cell-MHC interaction triggers T cell activation against the antigen, as well as and host cell activation via NFκB signaling in the infected cell which results in a feedback loop of remnant proviral genes and increased expression of antigenic peptides to increase the likelihood of recognition by activated T cells (FIG. 5B).

FIG. 6 depicts a non-limiting diagrammatic representation illustrating an exemplary T cell stimulation strategy for CRISPR/Cas-mediated HIV genome excision and interference RNA-based disruption of HIV Nef leading to the inhibition of MHC suppression and HIV peptide presentation for T cell-mediated response and cell-to-cell interference RNA spread.

### DETAILED DESCRIPTION

The present disclosure relates to, in part, allogeneic, hypoimmunogenic modified cell-derived biomimetic nanovesicles (BioNVs) that find use in treating HIV infection. In embodiments, BioNVs are targeted to HIV-infected cells with a surface-oriented targeting agent (e.g., viral epitope recognition receptors (VERRs), viral ligand/receptor complex, CAR, etc.), where the targeting agent can recognize a single target or multiple targets through a binding moiety for a desired/specific biomarker. The binding moiety can comprise all variations of an antibody construct, including for example, Fab, Fab′, Fab′-SH, F(ab′)2, scFv, diabody, nanobody, linear antibody, bispecific antibody, multi-specific antibody, chimeric antibody, humanized antibody, human antibody, and fusion proteins comprising the antigen-binding portion of an antibody, V_{H}H nanobodies, V_{NARS}, among other antibody formats, allowing BioNVs to target of any cell of interest (targeting any type of cell surface biomarker). In embodiments, the BioNV generated from the hypoimmunogenic modified cell is on the order of 20-1200 nm in size, far smaller in size than a traditional cell-based T-cell/NK-cell therapy. In embodiments, the plasma membrane-derived BioNVs retain the hypoimmunogenic properties of the modified cell (e.g., iPSC), which are conferred by genetic engineering focused on knock-out of specific immunogenic cell surface markers (e.g., MHC class I/II proteins, T cell receptor (TCR) proteins, cytokine release syndrome (CRS) proteins, etc.) and/or expression/overexpression of immunoprotective cell surface markers (e.g., CD47, CD34, CD24, CD200, α-phagocytic intagrins, etc.). In embodiments, the BioNVs can deliver one or more gene editing payloads by encapsulating, for example, one or more guide RNAs (gRNAs), site-specific endonucleases, and interference RNAs (RNAi).

Moreover, the present disclosure relates to, in part, methods of treatment, prevention, or amelioration of an HIV infection, especially in instances of latent viral infection, using the present BioNV compositions. For example, in embodiments, the method includes administering a BioNV that is targeted against immune cell surface receptor, Siglec-1, in a subset of latently HIV-infected immune cell subsets in PLWH, regardless of ART or checkpoint inhibitor status. In embodiments, the BioNV encapsulates a gene editing payload that includes at least two gRNAs, a first gRNA targeted against an HIV Gag proviral genomic sequence, and a second gRNA targeted against an HIV Nef proviral genomic sequence. In embodiments, the BioNV-delivered gene editing payload is capable of excising the HIV proviral genomic sequence between the HIV Gag proviral genomic sequence and the HIV Nef proviral genomic sequence, where an HIV 5′ LTR sequence, TAR Loop sequence, at least a portion of the HIV Gag proviral genomic sequence, and at least a portion of the HIV Nef proviral genomic sequence is not excised from the latently HIV-infected cell. In embodiments, the method of treatment, prevention, or ameliorating the HIV infection also includes administering an interference RNA (RNAi), wherein the RNAi is targeted against a mRNA sequence complementary to the portion of the HIV Nef proviral genomic sequence that is not excised (e.g., the RNAi is targeted again the HIV Nef mRNA sequence that was not excised). In embodiments, the RNAi is delivered by the BioNV. In embodiments, the delivery of the gene editing payload results in the HIV-infected cell expressing a partial Nef RNA sequence (pNef) from the portion of the HIV Nef proviral genomic sequence which reduces Nef-mediated MHC complex sequestration in the latently HIV-infected cell. In embodiments, the delivery of the gene editing payload also results in the HIV-infected cell expressing a partial Gag protein (pGag) from the portion of the portion of the HIV Gag proviral genomic sequence. In embodiments, the pGag is recognized by an MHC complex, resulting in T-cell stimulation against the pGag antigen expressed by the HIV-infected cell. In embodiments, pNef and pGag expression in the HIV-infected cell results in cell-to-cell spread of the RNAi via the SIDT-1/2 complex, and T-cell mediated cytotoxicity against one or more latently HIV-infected cells.

Those skilled in the art, upon review of this disclosure in its entirety, will appreciate that the methods of treatment, prevention, or amelioration of an HIV infection, can be performed without the interference RNA. In embodiments, the methods include administering a BioNV which delivers at least two gRNAs, where a first gRNA is targeted against an HIV Gag proviral genomic sequence, and a second gRNA is targeted against an HIV proviral 3′ LTR genomic sequence. In embodiments, the gene editing payload is capable of excising the HIV proviral genomic sequence between the HIV Gag proviral genomic sequence and the HIV 3′ LTR proviral genomic sequence. This can result, in embodiments, in an HIV 5′ LTR sequence, TAR Loop sequence, at least a portion of the HIV Gag proviral genomic sequence, and at least a portion of the HIV 3′ LTR proviral genomic sequence that is not excised from the HIV-infected cell. In embodiments, this method results in the HIV-infected cell expressing a partial Gag protein (pGag) from the portion of the HIV Gag proviral genomic sequence that was not excised. In embodiments, the pGag is recognized by an MHC complex, resulting in T-cell stimulation against the pGag antigen expressed by the HIV-infected cell.

In embodiments, BioNVs and methods of using them described herein are a delivery mechanism that overcomes the shortcomings of AAVs, LNPs, and exosome-based delivery systems in treating HIV. Current gene editor approaches focus on excising the entire translatable genome of HIV from the host cell, and requiring delivery of a gene editing therapeutic to all potentially infective (i.e., activatable) latent HIV-infected cells in the patient. This necessitates close monitoring over the course of treatment, further requiring robust diagnostics to narrow the detection window of the virus beyond that of ART suppression. When the virus rebounds, the patient quickly relapses to original viral loads, leading to inevitable redosing. If the patient is a human immunodeficiency virus controller (HIC), the immunologic mechanisms that originally contributed to viral equilibrium will likely be lost upon rebound from the treatment. In the least, the LNP method potentially allows for superior redosing over AAV delivery methods, but tropism, tissue penetration (biodistribution), and stability challenges remain.

In embodiments, methods described herein have advantages over current methods of direct treatment of integrated, latent virus. Current methods are inadequate to effectively clear (or guarantee clearance) of the virus, outside of a very limited HIC group that makes up 0.25%-0.5% of the patients that have been on ART therapy for more than 10 years (˜1250-2500 patients only in the US). Identifying these patients for effective and eligible clinical outcomes is challenging and expensive. In embodiments, co-treatment methods (alongside the gene editor excision), which stimulate the immune system to the site(s) of infection, as well as protect immune cells from re-infection, can be used with BioNVs.

In embodiments, co-therapeutic approaches include protecting immune cells from re-infection by alteration of the CCR5 receptor in the delta region of the protein receptor complex, using gene editing (e.g., CRISPR-based) approaches. In embodiments, the gRNAs can be designed based on mathematical models that can identify HIV sequence variants, for example as described in Chung et al., 2021, which is incorporated by reference in its entirety (Chung, et al. “Computational Design of gRNAs Targeting Genetic Variants Across HIV-1 Subtypes for CRISPR-Mediated Antiviral Therapy.” Front. Cell. Infect. Microbiol. Vol. 11, No. 593077, 2021:1-14). In embodiments, altering the CCR5 receptor on immune cells (e.g., T-cells and macrophages) can be performed by engineering the cells ex vivo and then injecting them into the patient in vivo by delivering (e.g., as a co-delivered gRNA) the CRISPR editors and gRNAs to the cell systemically in the patient.

In embodiments, co-therapeutic approaches include stimulation of the immune system by delivering a gene that expresses a co-stimulatory cytokine such as IL-15, or the addition of immune stimulating small molecule compounds, or altering the immune cells in manner that causes them to over-express certain proteins and receptors. In embodiments, co-therapeutic approaches include co-therapeutic gene excision using a gene editor with a cell-based therapy. In embodiments, co-therapeutic approaches include latency reactivating agents (LRAs) and antiretroviral therapy (ART).

### Biomimetic Nanovesicle (BioNV) for Treating HIV

In aspects, the present disclosure includes a biomimetic nanovesicle (BioNV) for treating HIV, wherein the BioNV comprises a targeting agent that is targeted against at least a first cell surface marker of a latently HIV-infected cell, and wherein the BioNV is configured to deliver a gene editing payload comprising at least two gRNAs.

In aspects, the present disclosure includes a biomimetic nanovesicle (BioNV) for treating HIV wherein the BioNV comprises a targeting agent that is targeted against at least a first cell surface marker of a latently HIV-infected cell, and wherein the BioNV is configured to deliver: a) a gene editing payload comprising at least two gRNAs; and b) a small RNA.

BioNVs used in methods herein are, in embodiments, derived from a hypoimmunogenic modified cell, such as a stem cell, an induced pluripotent stem cell (iPSC), a reprogrammed pluripotent or multipotent cell, an embryonic stem cell, a mesenchymal stem cell, or a differentiated cell which originates from any modified cell thereof. In embodiments, the modified cell is an iPSC. In embodiments, the iPSC is differentiated into a particular cell. In embodiments, the differentiated cell is a myeloid lineage cell (e.g., a macrophage, monocyte, neutrophil, etc.). In embodiments, the differentiated cell is a lymphoid lineage cell (e.g., a T cell, helper T cell, T-memory cell, NK cell, etc.) (Wang, et al. “3D-organoid culture supports differentiation of human CAR+ iPSCs into highly functional CAR T cells.” Cell Stem Cell. Vol. 29, 2022: pp. 515-527). In embodiments, the BioNV is produced from a γδ T call. In embodiments, BioNVs inherit barrier-crossing functionality of the cells from which they are derived, for example and without limitation, blood-brain barrier crossing functionality from macrophages or monocytes. This allows for, in embodiments, BioNVs to penetrate tissues to target HIV-infected cells.

In embodiments, the hypoimmunogenic modified cell is created by knocking-out, silencing, inactivating, blocking or otherwise negating the expression, transcriptional efficiencies, and/or activity of one or more immunogenic molecules. In embodiments, the hypoimmunogenic modified cell, and thus the BioNV derived therefrom, substantially lacks one or more MHC class I proteins, MHC class II proteins, HLA proteins, TCR proteins, and/or CRS proteins.

In embodiments, the BioNV has reduced or ablated β2-macroglobuin (B2M) protein expression and/or activity and/or MHC class I protein expression and/or activity, such as in the case for CD8+ T cell lineages. In embodiments, the BioNV has reduced or ablated CIITA protein expression and/or activity and/or MHC class II protein expression and/or activity, such as in the case of CD4+ T cell lineages. Without wishing to be bound by theory, these proteins contribute to the human leukocyte antigen (HLA) immunogenicity that requires HLA allele matching in the donor-recipient for treatment by cell-based therapies. In embodiments, allogeneic and/or hypoimmunogenic properties are achieved by reducing or ablating the expression and/or activity of the genes encoding the T cell receptor (TCR) proteins including, for example, the α and β chains (as in the case of αβ T cells) or the γ and δ chains (as in the case of γδ T cells) forming the ligand-binding site and the signaling modules CD3δ, CD3γ, CD3ε, and CD3ζ. In embodiments, this is performed to reduce extraneous T cell receptor types other than those of the targeting agent cassette and to further improve the homogeneity of the targeting agent of interest and reduce off-target effects from BioNV administration.

In embodiments, the presence of B2M reduces the number of potential doses to be administered due to the risk of preventing long term acceptance of the BioNVs by the recipient, such as what has been observed in the whole cell-based therapeutics. To overcome this issue, in embodiments, the HLA-E or HLA-G gene remains intact, allowing the immune system to adapt to the resulting BioNV. In embodiments, the HLA-A, HLA-B, HLA-C, HLA-F, HLA-E or HLA-G (but not both) are knocked out sequentially.

In embodiments, the BioNV has reduced or ablated HLA-A protein expression and/or activity. In embodiments, the BioNV has reduced or ablated HLA-B protein expression and/or activity. In embodiments, the BioNV has reduced or ablated HLA-C protein expression and/or activity. In embodiments, the BioNV has reduced or ablated HLA-E or HLA-G protein expression and/or activity. In embodiments, the BioNV has reduced or ablated HLA-F protein expression and/or activity.

In embodiments, BioNVs lack MHC class I and MHC class II complexes by knocking out (in the modified cell) of critical proteins involved in their expression, for example, β2-macroglobulin (B2M) which is a serum protein found in association with the MHC class I heavy chain on the surface of nearly all nucleated cells that is involved in the presentation of peptide antigens to the immune system. In embodiments, the allogeneic iPSCs have their CIITA gene disrupted, which is the Master Control Transcription Factor that regulates the expression of all MHC II genes. In embodiments, allogeneic iPSCs have their CIITA gene disrupted, which is the master control transcription factor that regulates the expression of all MHC II genes so that a resulting differentiated cell line (e.g., DCs, mononuclear phagocytes, endothelial cells, thymic epithelial cells, B cells, etc.) does not express or has reduced expression of MHC class II proteins.

In embodiments, the BioNV has reduced or ablated T cell alpha constant (TRAC) protein expression and/or activity. In embodiments, the BioNV has reduced or ablated T cell beta constant (TRBC) protein expression and/or activity. In embodiments, the BioNV has reduced or ablated PD-1 protein expression and/or activity; or wherein the BioNV has PD-1 protein expression and/or activity.

CRS is a major concern with whole cell therapies, where despite engineered hypoimmunogenicity, effector functions and other consequences of interaction with cells post-infusion can result in the release of biomolecules that result in a systemic inflammatory syndrome characterized by fever, multiple organ dysfunction, etc. In embodiments, the BioNV lacks one or more proteins that contribute to CRS. In embodiments, the BioNV has reduced or ablated protein expression and/or activity of CRS-related cytokines.

In embodiments, the BioNV has reduced or ablated IL-4 protein expression and/or activity. In embodiments, the BioNV has reduced or ablated IL-6 protein expression and/or activity. In embodiments, undesirable IL-6 packaging into the BioNV can result in the BioNV's contribution to a localized (and concentrated due to biomarker targeting) and/or potentially systemic CRS events. In embodiments, the BioNV has reduced or ablated IL-10 protein expression and/or activity. In embodiments, the BioNV has reduced or ablated IL-16 protein expression and/or activity. In embodiments, the reduction or ablation of interleukins decreases the likelihood of CRS.

In embodiments, BioNVs originate from cells modified to be hypoimmunogenic due to expression and/or activity of one or more immunoprotective proteins. In embodiments, the one or more immunoprotective proteins prevent or reduce an immune response in the subject, prevent or reduce premature clearance of the BioNV in the subject, prevent or reduce phagocytosis, confer barrier-crossing functionality, and the like, such as without limitation CD47, CD24, CD200, CD34, CCL2, H2-M3, MFG-E8, PD-L1 (in non-activated cell sources), CTLA-4, etc.

Serine proteinase inhibitor B9 (SerpinB9) is a member of the serine protease inhibitor superfamily. In embodiments, SerpinB9 protects cells from the immune killing effects of granzyme B. In embodiments, the BioNV is not designed to deliver granzyme and the BioNV has SerpinB9 expression and/or activity. In embodiments, the expression of SerpinB9 sequesters the function of granzyme B which is related to immunostimulatory responses, such as apoptosis of a targeted and/or infected cell.

In embodiments, the BioNV has reduced and/or ablated protein expression and/or activity of SerpinB9 (e.g., in BioNVs intended to deliver granzyme and/or perorin). In embodiments, the BioNV encapsulates one or more perorin molecules. In embodiments, the BioNV encapsulates one or more granzyme molecules. In embodiments, the granzyme molecules are selected from granzyme A, B, H, K, and M. In embodiments, the BioNV encapsulates one or more perforin molecules and/or one or more granzyme molecules derived from a cell from which the BioNV is derived. In embodiments, the BioNV encapsulates one or more perforin molecules and one and/or more granzyme molecules exogenously added to the BioNV.

In embodiments, the BioNV has CD34 protein expression and/or activity. In embodiments, the BioNV has CCL2 protein expression and/or activity. In embodiments, the BioNV has PD-L1 protein expression and/or activity. In embodiments, the BioNV has H2-M3 protein expression and/or activity.

In embodiments, the BioNV has CD47 protein expression and/or activity. Exosomes and cell-derived vesicles (CDVs) are readily cleared from the body by macrophages through phagocytosis. Phagocytosis greatly impacts the therapeutic value and efficacy of CDVs. To prevent macrophage depletion of BioNVs, but without wishing to be bound by theory, in embodiments, the BioNV has CD47 tagged on the surface. A CD47tg (tag) provides a “do not eat me” signal which, in embodiments, increases the half-life and serum stability of the BioNV in the subject. In embodiments, the molecular CD47 isoform 2 (the isoform that interacts with the SIRPα receptor on a macrophage) is engineered into the modified cell (e.g., iPSC cell line). Without the CD47tg, the BioNV half-life would be diminished due to phagocytosis inhibition, resulting in the need for higher and/or more frequent doses. In embodiments, prevention of the potential inhibitory phenotypes of CD47 expression across cells is done via interference with the inhibitory mechanism of action of the series of microRNAs on the 3′UTR of the CD47 gene by deleting this region in stable constructs or by eliminating/inhibiting the expression of the microRNAs. In embodiments, this can resolve inhibitory issues caused by the microRNAs across differentiated cell subsets.

In embodiments, the BioNV has CD24 protein expression and/or activity. CD24 is a sialoglycoprotein expressed on mature granulocytes and B-cells and is also an anti-phagocytic protein. CD24 prevents phagocytosis through interactions with Siglec-G/10 on macrophages. In embodiments, the BioNV has a chimeric CD24/CD47 protein expression and/or activity. In embodiments, the BioNV expresses a chimeric CD24/CD47 with a tethered transmembrane domain. In embodiments, the domains of CD47 isoform 2 and CD24 can be either separately expressed or tethered to form a bilobed, chimeric protein. In embodiments, the BioNVs are derived from iPSCs originating from fibroblasts, not ABO cells.

In embodiments, the BioNV has CD200 protein expression and/or activity. In embodiments, CD200 tags minimize phagocytosis by macrophages and also prevent the activation of granulocytes. In embodiments, if a CD47 or CD24 tag is used, or a CD24/CD47 chimeric, bilobed protein tag (each prevents phagocytosis) in combination with overexpressed H2-M3 (which dampens the NK response), stability can be achieved without CD200, while allowing adequate BioNV clearance. In embodiments, CD200 can be expressed to prevent the activation of granulocytes, while eliminating a CD47 tag or a CD24 tag, but not both tags. In embodiments, the BioNV has chimeric CD24/CD200 protein expression and/or activity or chimeric CD47/CD200 protein and/or activity.

In embodiments, the BioNV does not express and/or possess activity of all three of CD47, CD24, and CD200. In embodiments, the BioNV is engineered such it is stabilized in the subject, but not to a degree where the BioNVs are resistant to being cleared from the body. A BioNV that is too stable could eventually trigger a humoral response, resulting in limiting the number of doses or treatments that can be administered.

In embodiments, the BioNV has CTLA-4 protein expression and/or activity. In embodiments, the BioNV has MFG-E8 protein expression and/or activity. In embodiments, the BioNV has NCAM protein expression and/or activity. In embodiments, the BioNV has a-phagocytic integrin protein expression and/or activity. In embodiments, the BioNV the BioNV has FasL protein expression and/or activity.

In embodiments, the BioNV has reduced or ablated expression and/or activity of one or more immunogenic proteins, such as, proteins that result in an immune response in the subject, donor-recipient mismatch, HLA alloimmunity, inflammation, CRS, and the like, for example and without limitation, MHC class I proteins, MHC class II proteins, HLA proteins, TCR proteins, CRS proteins, etc. Persons skilled in the art, with the benefit of this disclosure in its entirety, will understand the various assays that can be used to determine if a protein (or BioNV) is immunogenic and/or will be immunogenic if provided to a subject. For example, in non-limiting embodiments, immunogenic proteins (and BioNVs herein) can be determined for their ability to be immunogenic by using immunogenicity assessment assays to assess the ability to bigger cytokine release in cells, T cell and/or B cell response assays, detection of anti-drug antibody (ADA) generation, antigen-induced PBMC proliferation assay, etc. In embodiments, the BioNV has reduced or ablated protein expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

In embodiments, the BioNV has expression and/or activity of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, the BioNV is allogeneic. In embodiments, the BioNV does not cause an adverse immune reaction in subjects to which it is administered.

In embodiments, the BioNV substantially lacks protein and/or activity of HLA-A, HLA-B, HLA-C, HLA-F, CIITA, IL-6, TRAC, TRBC, and one of either HLA-E or HLA-G.

In embodiments, the BioNV substantially lacks protein and/or activity of HLA-A, HLA-B, HLA-C, HLA-F, CIITA, IL-6, TRAC, TRBC, PD-1, and one of either HLA-E or HLA-G.

In embodiments, the BioNV substantially lacks protein and/or activity of HLA-A, HLA-B, HLA-C, HLA-F, CIITA, IL-6, TRAC, TRBC, PD-1, one of either HLA-E or HLA-G, and one or more of IL-4, IL-10, and IL-16.

In embodiments, the BioNV has membrane-embedded a-phagocytic integrin, CCL2, H2-M3, FasL, MFEG8, and PD-L1 and/or CTLA-4; and one of either CD24, CD47, CD200, chimeric CD24/CD47, chimeric CD24/CD200, and chimeric CD47/CD200, or two of either CD24, CD47, and CD200.

In embodiments, the BioNV has membrane-embedded a-phagocytic integrin, CCL2, H2-M3, FasL, MFEG8, SerpinB9, and PD-L1 and/or CTLA-4; and one of either CD24, CD47, CD200, chimeric CD24/CD47, chimeric CD24/CD200, and chimeric CD47/CD200, or two of either CD24, CD47, and CD200.

In embodiments, the BioNV has membrane-embedded CD200 protein and substantially lacks protein of either CD24 or CD47.

In embodiments, the BioNVs comprise one or more targeting agents directed to one or more surface markers of a latently HIV-infected cell. In embodiments, the targeting agent is one or more of a CAR, VERR, viral ligand, viral receptor, or an antibody or antibody format selected from a monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody. In embodiments, the VERR or viral ligand is a gp120/gp41 complex (e.g., including a detoxified gp120/gp41 complex). In embodiments, the targeting agent is the scFv. In embodiments, the targeting agent is the CAR.

In embodiments, the CAR comprises a transmembrane domain derived from CD28, CD3ζ, CD4, CD8α, ICOS, or fragment and/or combination thereof. In embodiments, the CAR comprises an intracellular domain further comprising an intracellular signaling domain of a CD3ζ-chain and/or one or more co-stimulatory molecules, optionally selected from CD28, 4-1BB, ICOS, CD27, and OX40. In embodiments, the CAR is activated, optionally wherein the CAR is activated via its target, through another receptor, and/or a virus.

In embodiments, the BioNV has a VERR ligand-binding ectodomain or viral ligand (cell receptor ligand), CD8 or IgG4-derived hinge, transmembrane domain, co-stimulatory molecule(s) (e.g., CD27, CD28, ICOS, 4-1BB, and/or OX40), and/or stimulatory molecule (e.g., CD3ζ chain or FcRγ chain).

In embodiments, the BioNVs has a VERR and/or viral ligand fused with Vp1 AAV protein ectodomain, transmembrane domain, and PLA2 domain from AAV (primed). The PLA2 domain can be present on the external, solvent-exposed surface of the BioNV in a non-activated form. Alternatively, or additionally, the PLA2 domain can be engineered to become active from the inside of the BioNV.

In embodiments, the BioNV has a surface-exposed transmembrane anchored membrane fusion protein(s) fused to internally oriented non-active (or activated) lipid fusion domains (e.g., PLA2).

In embodiments, the BioNV has an externally-oriented transmembrane anchored membrane lipid fusion protein and/or lipid protein complexes. In embodiments, the BioNVs are capable of fusion with the plasma membrane of target cells, resulting in the direct injection or deposit of the payload into the cytoplasm of the cell. In embodiments, the VERR or viral ligand can have a generic internal coiled trimer which is linked to a PLA2 protein of the lipid protein complex. Upon binding of the VERR/viral ligand to the target, the coiled trimer can undergo a conformational change which activates the fusion protein (or protein complex) and initiates the internalization mechanism(s) of the target cell.

In embodiments, the BioNV has a surface expressed, detoxified HIV gp120/gp41. In embodiments, BioNVs expressing detoxified gp120/gp41 complexes can also facilitate fusion with the plasma membrane of a target cell. WT gp120/gp41 complexes on BioNVs can target CD4/CXCR5 receptors on latently HIV-infected lymphocytes to deliver therapeutic payloads, for example CRISPR/Cas-based gene editing machinery, gRNA, and RNAi into the cytoplasm. In embodiments, the “detoxified HIV gp120/gp41” retains the delivery function of the WT, but has one or more mutations which reduce its cellular toxicity. Without wishing to be bound by theory, in embodinest, this delivery mechanism has the advantage of evading the canonical endosomal processing pathways. In embodiments, surface epitopes of the gp120 receptor ligand can be mutated to target cellular markers other than CD4/CXCR5, increasing the treatment repertoire.

In embodiments, the BioNV includes a detoxified gp120/gp41 complex to deliver the gene editing payload. In embodiments, the complex can be expressed on the surface of the cell (transmembrane) and be surface-exposed in the resulting BioNV (post-processing). In embodiments, the gp120/gp41 complex recognizes CD4/CCR5 receptors on target cells and can be used to deliver a gene editor to cells that expresses these receptors. In embodiments, the detoxified gp120/gp41 receptor complex is used to treat immune cells with viral infection because the complex combines a mechanism to recognize the target cells (via gp120 interacting with CD4 receptors) and a mechanism for injecting the gene editor into the cytoplasm, thereby avoiding the less efficient endosomal pathway, via the gp41 interacting with the CCR5 receptor. In embodiments, gp120 is the targeting portion of the complex and gp41 is the harpooning/fusogen portion of the complex. In embodiments, the gp120/gp41 complex is highly precise and there is reduced off-target delivery to unintentional cells.

In embodiments, the gp120/gp41 complex BioNVs are administered at higher doses for latently infected cells. In embodiments, latently infected cells are targeted with Siglec, PD-1, CD4, CCR5, CD32a, CD91, CD257, LAG-3, CD147, CD231, cell adhesion molecule 1 (CEACAM1), and/or plexin B2 (PLXNB2) (or a combination thereof) as transmembrane proteins in a construct in the BioNV to target a broader cell population.

In embodiments, the fusion of BioNVs to a cell is mediated by the proteins as described herein.

In embodiments, the BioNV has a targeting agent that is targeted against at least a first cell surface marker of a latently HIV-infected cell. In embodiments, the BioNV can have more than one targeting agent and/or a targeting agent, such as a bispecific CAR which can target a first cell surface marker and a second cell surface marker of a latently HIV-infected cell. In embodiments, the marker is expressed on one or more of T cells, dendritic cells, and macrophages, or any cell type in an HIV-infected cell reservoir. The T cells can be CD4+ T cells or CD8+ cytotoxic T cells (CTLs). The marker can be expressed by immune cells, including any lymphoid progenitor lineage (e.g., T cells subsets such as Tregs, Th17, Th2, Th1, Th0, and Th22), or any myeloid progenitor lineage (e.g., mast cells, myoblast, monocytes, eosinophils, basophils, neutrophils, DCs, and macrophages).

In embodiments, the first cell surface marker is or comprises a sialic acid-binding immunoglobulin-like lectin (Siglec) molecule. In embodiments, the Siglec is or comprises Siglec-1. In embodiments, in the context of targeting latently HIV-infected immune cells, sialic acid-binding immunoglobulin-like lectins (Siglecs), such as Siglec-1, can be the effective target for the methods of treatment. Siglec targets can be divided into subsets based on sequence and structure similarity, such as CD33-related Siglecs (e.g., Siglec-H, Siglec-5, and Siglec-14) and CD22-related Siglecs.

In embodiments, the first cell surface marker is or comprises CD32a (also known as FcγRIIa). In embodiments, targeting CD32a cell surface expression of the low affinity Fc receptor CD32a can allow targeting of the replication-competent HIV-1 reservoir in CD4+ T cells of HIV-1-infected participants receiving suppressive antiretroviral therapy (ART). In embodiments, targeting CD32a biomarker is used to deliver a payload, e.g., a gene editor to non-infected cells. In embodiments, BioNVs (e.g., CD32a-targeted BioNVs) carry a gene editing payload which is not active, or does not function, in non-HIV-infected cells. In embodiments, the BioNV is targeted against at least a portion of CD32a (surface receptor glycoprotein also known as FcγRIIa). In embodiments, methods herein target HIV infected cells and/or reservoirs of HIV proviruses in cells that express CD32a. CD32a is a cell surface marker whose expression has been associated with latent HIV-1 infection (BELSHAN et al. 2021) and (DESCOURS et al., “CD32a is a marker of a CD4 T-cell HIV reservoir harbouring replication-competent proviruses,” Nature, Vol. 543, 2017:564-7). In embodiments, the BioNV is targeted against at least a portion of CD32a and at least one other cell surface marker (e.g., via a bispecific CAR).

In embodiments, the marker for methods herein is, or comprises, HLA-DR. In embodiments, methods targeting HLA-DR allow targeting of the replication-competent HIV-1 reservoir in participants receiving suppressive anti-retroviral therapy (ART). In embodiments, methods targeting a HLA-DR biomarker are used to deliver a payload, e.g., a gene editor to non-infected cells. In embodiments, HLA-DR-targeted BioNVs carry a gene editing payload which is not active in non-virally infected cells. In embodiments, methods herein target HIV infected cells and/or reservoirs of HIV proviruses in cells that express a canonical marker of activation, HLA-DR (MHC class II). HLA-DR expression has been shown to be associated with cells latently harboring HIV proviral sequences, for example as described in (COHN L B, et al., “The Biology of the HIV-1 Latent Reservoir and Implications for Cure Strategies,” Cell Hos Microbe, Vol. 27, No. 4, 2020: pp. 519-30), (HORSBURGH B A, et al., “High levels of genetically intact HIV in HLA-DR+ memory T cells indicates their value for reservoir studies,” AIDS. Vol. 34, No. 5, 2020: 659-668) and (LEE E, et al., “Memory CD4+ T-Cells Expressing HLA-DR Contribute to HIV Persistence During Prolonged Antiretroviral Therapy,” Front Microbiol, Vol. 10, No. 2214, 2019: pp. 1-19). In embodiments, the BioNV is targeted against at least a portion of HLA-DR and at least one other cell surface marker (e.g., via a bispecific CAR).

In embodiments, the marker for methods herein is, or comprises, CD25 (the alpha chain of the IL-2 receptor and a constitutive marker of regulatory T cells). In embodiments, methods targeting CD25 allow targeting of the replication-competent HIV-1 reservoir in participants receiving suppressive anti-retroviral therapy (ART). In embodiments, methods targeting a CD25 biomarker are used to deliver a payload, e.g., a gene editor to non-infected cells. In embodiments, CD25-targeted BioNVs carry a gene editing payload which is not active in non-virally infected cells. In embodiments, methods herein target HIV infected cells and/or reservoirs of HIV proviruses in cells that express CD25. CD25 expression has been shown to be associated with cells latently harboring HIV proviral sequences, for example as described in (COHN, et al. 2020) and (TRAN T A, et al., “Resting regulatory CD4 T cells: a site of HIV persistence in patients on long-term effective antiretroviral therapy,” PLoS One. Vol. 3, No. 10, 2008: pp. 1-11). In embodiments, the BioNV is targeted against at least a portion of CD25 and at least one other cell surface marker (e.g., via a bispecific CAR).

In embodiments, the marker for methods herein is, or comprises, CD69 (a marker of tissue-resident memory T cells). In embodiments, methods targeting CD69 allow targeting of the replication-competent HIV-1 reservoir in participants receiving suppressive anti-retroviral therapy (ART). In embodiments, methods targeting a CD69 biomarker are used to deliver a payload, e.g., a gene editor to non-infected cells. In embodiments, CD69-targeted BioNVs carry a gene editing payload which is not active in non-virally infected cells. In embodiments, methods herein target HIV infected cells and/or reservoirs of HIV proviruses in cells that express CD69. CD69 expression has been shown to be associated with cells latently harboring HIV proviral sequences, for example as described in (COHN, et al. 2020) and (CANTERO-PÉREZ J, et al., “Resident memory T cells are a cellular reservoir for HIV in the cervical mucosa,” Nat Commun. Vol. 10, No. 1, 2019: pp. 1-16). In embodiments, the BioNV is targeted against at least a portion of CD69 and at least one other cell surface marker (e.g., via a bispecific CAR).

Methods herein, in embodiments, target subsets of cells that express HLA-DR, CD25, and/or CD69. Methods herein, in embodiments, target subsets of cells that do not fulfill the classical definition of resting CD4+ T cells which can contribute to the long-term persistence of HIV during ART.

In embodiments, the marker for methods herein is, or comprises, one or more of programmed cell death-1 (PD-1), cytotoxic T-lymphocyte-associated protein 4 (CTLA-4), T cell immunoglobulin and ITIM domain (TIGIT), lymphocyte activation gene 3 (LAG-3), T cell immunoglobulin and mucin 3 (TIM-3) and/or CD160. In embodiments, methods targeting one or more of PD-1, CTLA-4, TIGIT, LAG-3, TIM-3 and/or CD160 allow targeting of the replication-competent HIV-1 reservoir in participants receiving suppressive anti-retroviral therapy (ART). In embodiments, methods target one or more of a PD-1, CTLA-4, TIGIT, LAG-3, TIM-3 and/or CD160 biomarker to deliver a payload, e.g., a gene editor to non-infected cells. In embodiments, PD-1, CTLA-4, TIGIT, LAG-3, TIM-3 and/or CD160-targeted BioNVs carry a gene editing payload which is not active in non-virally infected cells. In embodiments, methods herein target HIV infected cells and/or reservoirs of HIV proviruses in cells that express PD1, CTLA-4, TIGIT, LAG-3, TIM-3, and/or CD160. PD-1, CTLA-4, TIGIT, LAG-3, TIM-3, and/or CD160 expression has been shown to be associated with cells latently harboring HIV proviral sequences, for example as described in (COHN, et al. 2020), (CHEW G M, et al., “TIGIT Marks Exhausted T Cells, Correlates with Disease Progression, and Serves as a Target for Immune Restoration in HIV and SIV Infection,” PLoS Pathog, Vol. 12, No. 1, 2016: 1-28), (FROMENTIN R, et al., “CD4+ T Cells Expressing PD-1, TIGIT and LAG-3 Contribute to HIV Persistence during ART,” PLoS Pathog, Vol. 12, No. 7, 2016: 1-19), and (PARDONS M, et al., “Single-cell characterization and quantification of translation-competent viral reservoirs in treated and untreated HIV infection,” PLoS Pathog, Vol. 15, No. 2, 2019: pp. 1-28). In embodiments, the BioNV is targeted against at least a portion of PD-1, CTLA-4, TIGIT, LAG-3, TIM-3, and/or CD160 and at least one other cell surface marker (e.g., via a bispecific CAR).

In embodiments, the marker for methods herein is, or comprises, Cyclophilin B (CypB, also known as peptidyl prolyl isomerase B (PPIB), is a member of the cyclophilin family of immunophilins, which a cytosolic enzyme that can be enriched on the plasma membrane). In embodiments, methods targeting CypB allow targeting of the replication-competent HIV-1 reservoir in participants receiving suppressive anti-retroviral therapy (ART). In embodiments, methods targeting a CypB biomarker are used to deliver a payload, e.g., a gene editor to non-infected cells. In embodiments, CypB-targeted BioNVs carry a gene editing payload which is not active in non-virally infected cells. In embodiments, methods herein target HIV infected cells and/or reservoirs of HIV proviruses in cells that express CypB. CypB expression has been shown to upregulated during HIV replication, for example as described in (BELSHAN M, et al., “Discovery of candidate HIV-1 latency biomarkers using an OMICs approach,” Virology, Vol. 558, 2021: pp. 86-95), (DEBOER J, et al., “Alterations in the nuclear proteome of HIV-1 infected T-cells,” Virology, 468-470, 2014: pp. 409-420), and (DEBOER J, et al., “Cyclophilin B enhances HIV-1 infection,” Virology, Vol. 489, 2016: pp. 282-91). In embodiments, the BioNV is targeted against at least a portion of CypB and at least one other cell surface marker (e.g., via a bispecific CAR).

In embodiments, the marker for methods herein is, or comprises, Sec62 (an intracellular component of the SEC61 complex that functions in protein translocation in the endoplasmic reticulum). In embodiments, methods targeting Sec62 allow targeting of the replication-competent HIV-1 reservoir in participants receiving suppressive anti-retroviral therapy (ART). In embodiments, methods targeting a Sec62 biomarker are used to deliver a payload, e.g., a gene editor to non-infected cells. In embodiments, Sec62-targeted BioNVs carry a gene editing payload which is not active in non-virally infected cells. In embodiments, methods herein target HIV infected cells and/or reservoirs of HIV proviruses in cells that express Sec62. Sec62 expression has been shown to be upregulated during HIV replication and may exist in the cytoplasmic membrane of latent cells and/or interact with HIV-1 gp41, for example as described in (BELSHAN, et al. 2021) and (JAGER S, et al., “Global landscape of HIV-human protein complexes,” Nature, Vol. 481, 2012: pp. 365-370). In embodiments, the BioNV is targeted against at least a portion of Sec62 and at least one other cell surface marker (e.g., via a bispecific CAR).

In embodiments, the marker for methods herein is, or comprises, Rab10 (a member of the RAS superfamily of small GTPases involved in vesicular transport). In embodiments, methods targeting Rab10 allow targeting of the replication-competent HIV-1 reservoir in participants receiving suppressive anti-retroviral therapy (ART). In embodiments, methods targeting a Rab10 biomarker are used to deliver a payload, e.g., a gene editor to non-infected cells. In embodiments, Rab10-targeted BioNVs carry a gene editing payload which is not active in non-virally infected cells. In embodiments, methods herein target HIV infected cells and/or reservoirs of HIV proviruses in cells that express Rab10 in the cytoplasmic membrane. Rab10 overexpression is consistent with its presence in Staufen1-containing ribonucleoprotein complexes found in HIV-1 infected cells, for example as described in (BELSHAN, et al. 2021) and (MILEV M P, et al., “Characterization of staufen1 ribonucleoproteins by mass spectrometry and biochemical analyses reveal the presence of diverse host proteins associated with human immunodeficiency virus type 1,” Front Microbiol. Vol. 3, No. 367, 2012:1-21). In embodiments, the BioNV is targeted against at least a portion of Rab10 and at least one other cell surface marker (e.g., via a bispecific CAR).

In embodiments, the marker for methods herein is, or comprises, signal peptidase complex subunit1 (SPCS1) (SPCS1 is a cytoplasmic component of the microsomal signal peptidase complex that participates in protein processing and transport in the endoplasmic reticulum). In embodiments, methods targeting SPCS1 allow targeting of the replication-competent HIV-1 reservoir in participants receiving suppressive anti-retroviral therapy (ART). In embodiments, methods targeting a SPCS1 biomarker are used to deliver a payload, e.g., a gene editor to non-infected cells. In embodiments, SPCS1-targeted BioNVs carry a gene editing payload which is not active in non-virally infected cells. In embodiments, methods herein target HIV infected cells and/or reservoirs of HIV proviruses in cells that express SPCS1 in the cytoplasmic membrane. SPCS1 is involved in the assembly and budding of flaviviruses and its cellular expression has been associated with HIV-1 infection (BELSHAN et al. 2021). In embodiments, the BioNV is targeted against at least a portion of SPCS1 and at least one other cell surface marker (e.g., via a bispecific CAR).

In embodiments, the marker for methods herein is, or comprises, Bruton tyrosine kinase (BTK) (BTK is a tyrosine kinase involved in B cell development). In embodiments, methods targeting BTK allow targeting of the replication-competent HIV-1 reservoir in participants receiving suppressive anti-retroviral therapy (ART). In embodiments, methods targeting a BTK biomarker are used to deliver a payload, e.g., a gene editor to non-infected cells. In embodiments, BTK-targeted BioNVs carry a gene editing payload which is not active in non-virally infected cells. In embodiments, methods herein target HIV infected cells and/or reservoirs of HIV proviruses in cells that express BTK. BTK is a cell surface marker whose expression has been associated with latent HIV-1 infection (BELSHAN et al. 2021) and (BERRO et al., “Identifying the membrane proteome of HIV-1 latently infected cells,” J. Biol. Chem., Vol. 282, No. 11, 2007: 8207-18). In embodiments, the BioNV is targeted against at least a portion of BTK and at least one other cell surface marker (e.g., via a bispecific CAR).

In embodiments, the marker for methods herein is, or comprises, CD3 (T cell surface receptor complex). In embodiments, methods targeting CD3 allow targeting of the replication-competent HIV-1 reservoir in participants receiving suppressive anti-retroviral therapy (ART). In embodiments, methods targeting a CD3 biomarker are used to deliver a payload, e.g., a gene editor to non-infected cells. In embodiments, CD3-targeted BioNVs carry a gene editing payload which is not active in non-virally infected cells. In embodiments, methods herein target HIV infected cells and/or reservoirs of HIV proviruses in cells that express CD3. CD3 is a cell surface marker whose expression has been associated with latent HIV-1 infection (BELSHAN et al. 2021) and (IGLESIAS-USSEL, et al., “High levels of CD2 expression identify HIV-1 latently infected resting memory CD4+ T cells in virally suppressed subjects,” J Viral, Vol. 87, No. 16, 2013: pp. 9148-58). In embodiments, the BioNV is targeted against at least a portion of CD3 and at least one other cell surface marker (e.g., via a bispecific CAR).

In embodiments, the marker for methods herein is, or comprises, CD2 (T cell and NK cell surface receptor complex). In embodiments, methods targeting CD2 allow targeting of the replication-competent HIV-1 reservoir in participants receiving suppressive anti-retroviral therapy (ART). In embodiments, methods targeting a CD2 biomarker are used to deliver a payload, e.g., a gene editor to non-infected cells. In embodiments, CD2-targeted BioNVs carry a gene editing payload which is not active in non-virally infected cells. In embodiments, methods herein target HIV infected cells and/or reservoirs of HIV proviruses in cells that express CD2. CD2 is a cell surface marker whose expression has been associated with latent HIV-1 infection (DARCIS, et al., “The Quest for Cellular Markers of HIV Reservoirs: Any Color You Uke,” Front Immunol, Vol. 10, No. 2251, 2019: pp. 1-9) and (IGLESIAS-USSEL, et al., “High levels of CD2 expression identify HIV-1 latently infected resting memory CD4+ T cells in virally suppressed subjects,” J Virol, Vol. 87, No. 16, 2013: pp. 9148-58). In embodiments, the BioNV is targeted against at least a portion of CD2 and at least one other cell surface marker (e.g., via a bispecific CAR).

In embodiments, the marker for methods herein is, or comprises, CD20 (B cell surface receptor complex). In embodiments, methods targeting CD20 allow targeting of the replication-competent HIV-1 reservoir in participants receiving suppressive anti-retroviral therapy (ART). In embodiments, methods targeting a CD20 biomarker are used to deliver a payload, e.g., a gene editor to non-infected cells. In embodiments, CD20-targeted BioNVs carry a gene editing payload which is not active in non-virally infected cells. In embodiments, methods herein target HIV infected cells and/or reservoirs of HIV proviruses in cells that express CD20. CD20 is a cell surface marker whose expression has been associated with HIV RNA⁺ cells (DARCIS et al. 2019) and (SERRA-PEINADO et al., “Expression of CD20 after viral reactivation renders HIV-reservoir cells susceptible to Rituximab,” Nat Commun, Vol. 10, No. 3705, 2019: pp. 1-15). In embodiments, the BioNV is targeted against at least a portion of CD20 and at least one other cell surface marker (e.g., via a bispecific CAR).

In embodiments, the marker for methods herein is, or comprises, CD30 (overexpression associated with lymphomas; CD30 expression is triggered by viral infection). In embodiments, methods targeting CD30 allow targeting of the replication-competent HIV-1 reservoir in participants receiving suppressive anti-retroviral therapy (ART). In embodiments, methods targeting a CD30 biomarker are used to deliver a payload, e.g., a gene editor to non-infected cells. In embodiments, CD30-targeted BioNVs carry a gene editing payload which is not active in non-virally infected cells. In embodiments, methods herein target HIV infected cells and/or reservoirs of HIV proviruses in cells that express CD30. CD30 is a cell surface marker whose expression has been associated with HIV-infected CD4+ T cells (DARCIS et al. 2019), (BISWAS P, et al., “CD30 ligation differentially affects CXCR4-dependent HIV-1 replication and soluble CD30 secretion in non-Hodgkin cell lines and in gamma delta T lymphocytes,” Eur J Immunol. Vol. 33, No. 11, 2003: pp. 3136-45), (ROMAGNANI S, et al., “Role for CD30 in HIV expression,” Immunol Lett. Vol. 51, No. 1-2, 1996: pp. 83-8), (HOGAN L E, et al., “Increased HIV-1 transcriptional activity and infectious burden in peripheral blood and gut-associated CD4+ T cells expressing CD30,” PLoS Pathog, Vol. 14, No. 2, 2018: pp. 1-19), and (WANG C C, et al., “Transient loss of detectable HIV-1 RNA following brentuximab vedotin anti-CD30 therapy for Hodgkin lymphoma,” Blood Adv, Vol. 2, No. 23, 2018: pp. 3479-82). In embodiments, the BioNV is targeted against at least a portion of CD30 and at least one other cell surface marker (e.g., via a bispecific CAR).

In embodiments, the first cell surface marker is or comprises a Siglec, CD2, CD3, CD4, CCR5, CD20, CD25, CD30, CD32a, CD69, CD91, CD160, CD257, LAG-3, CD147, CD231, CEACAMI, PLXNB2, HLA-DR, PD-1, CTLA-4, TIGIT, LAG-3, TIM-3, BTK, Cyclophilin B, Sec62, Rab10, SPCS. In embodiments, the BioNV targets a combination thereof. In embodiments, the BioNV targeted one or more cell surface markers of latent infection.

In embodiments, the BioNV comprises a bispecific chimeric receptor, such as, without limitation, one or more antibody or antibody formats described herein, targeted against two or more of a Siglec, CD2, CD3, CD4, CCR5, CD20, CD25, CD30, CD32a, CD69, CD91, CD160, CD257, LAG-3, CD147, CD231, CEACAMI, PLXNB2, HLA-DR, PD-1, CTLA-4, TIGIT, LAG-3, TIM-3, BTK, Cydophilin B, Sec62, Rab10, SPCS. In embodiments, the BioNV comprises a bispecific chimeric receptor that targets a Siglec and CD32a. In embodiments, the BioNV comprises a bispecific chimeric receptor that targets CD4 and a co-receptor.

In embodiments, the BioNV encapsulates a gene editing payload; e.g., “lumen-loading”, or the ability of the BioNV to have a gene editing payload loaded into the lumen (space in the biomimetic nanovesicle). In embodiments, the payload is a gene editing payload comprising one or more gene editors. In embodiments, the one or more gene editors forms a complex with at least one of the two gRNAs.

In embodiments, the one or more gene editors is a gene editing nucleic acid and/or protein, such as for example, a site-directed endonudease, CRISPR/Cas nuclease, SpCas9-HF1, Cpf1, CasX, C2c1, C2c2, C2c3, Cas9, TevCas9, Archaea Cas9, CasY.1, CasY.2, CasY.3, CasY.4, CasY.5, CasY.6, Cas omega, transposase, and/or an ortholog or homolog thereof. In embodiments, the gene editor is a CRISPR/Cas nuclease. In embodiments, the gene editor is a site-directed endonudease. In embodiments, the one or more gene editors is a gene editing nucleic acid and/or protein including SpCas9-HF1, which is a high-fidelity Streptococcus pyogenes Cas9 (SpCas9) variant that has one or more alterations relative to the wild-type Cas9 designed to reduce non-specific DNA contacts, for example as described in Kleinstiver B P, et al. “High-fidelity CRISPR-Cas9 nucleases with no detectable genome-wide off-target effects,” Nature. 2016 Vol. 529, No. 7587, 2016: pp. 490-5, the entire contents of which are hereby incorporated by reference in its entirety.

In embodiments, the gene editors can also include gRNA, which, as used herein, refers to guide RNA. In embodiments, the guide RNA ranges in size between about 16 nucleotides (or mer) to about 100 nucleotides. In embodiments, the small RNA is at least about 16 mer, at least about 17 mer, at least about 18 mer, at least about 19 mer, at least about 20 mer, at least about 21 mer, at least about 22 mer, at least about 23 mer, at least about 24 mer, at least about 25 mer, at least about 26 mer, at least about 27 mer, at least about 28 mer, at least about 29 mer, at least about 30 mer, at least about 35 mer, at least about 40 mer, at least about 45 mer, at least about 50 mer, at least about 55 mer, at least about 60 mer, at least about 65 mer, at least about 75 mer, at least about 80 mer, at least about 85 mer, at least about 90 mer, at least about 95 mer, or at least about 100 mer or more in length.

In embodiments, the gRNA is a sequence complimentary to a coding or a non-coding sequence and can be tailored to the particular sequence to be targeted, including mutant sequences. In embodiments, the gRNA can be a sequence complimentary to a protein coding sequence, for example, a sequence encoding one or more viral structural proteins, e.g., gag, pdl, env, tat, or a portion of p1, p2, p6, p7, p17, p24, gp41, and/or gp120. In embodiments, the gRNA sequence can be a sense or anti-sense sequence. In embodiments, when a gene editor composition is administered herein, preferably without limitation, including two or more gRNAs; however, a single gRNA can also be used.

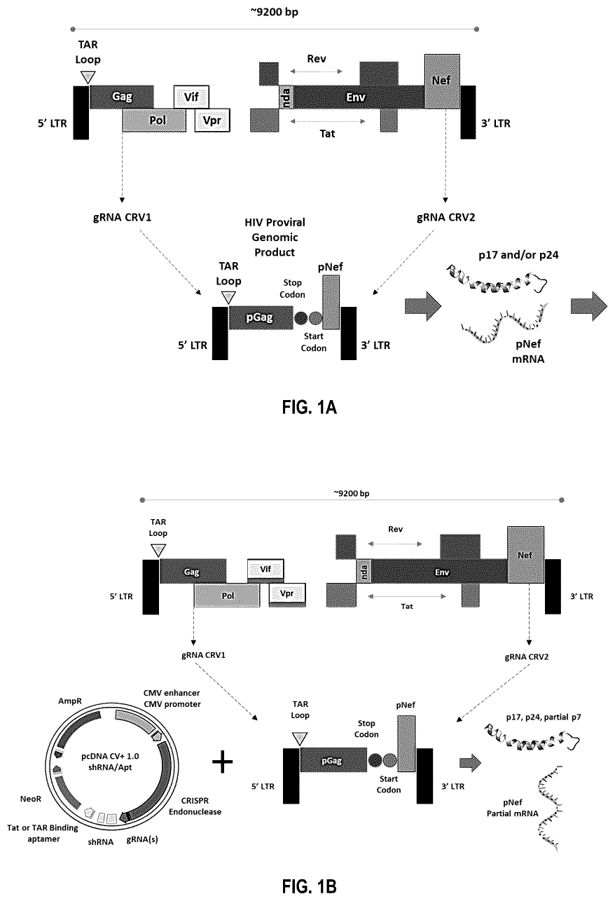

In embodiments, the one or more gene editors excises the HIV proviral genomic sequence between the at least two gRNA target sequences in the latently HIV-infected cell. For example, in embodiments, a first gRNA targeted against Gag forms a complex with a site-specific endonuclease (e.g., Cas enzyme) and catalyzes a double-stranded break in the DNA of an HIV infected cell with an integrated HIV genome (see e.g., FIG. 1A-B, gRNA CRV1); a second gRNA targeted against Nef forms a complex with a site-specific endonuclease (e.g., Cas enzyme) and catalyzes a double-stranded break in the DNA of an HIV infected cell with an integrated HIV genome (see e.g., FIG. 1A-B, gRNA CRV2). In embodiments, the resultant excision by the one or more gene editors retains in the latently HIV-infected cell an integrated 5′ LTR sequence, TAR Loop sequence, and an at least a portion of a Gag coding sequence (see e.g., FIG. 1A-B). Additionally, in embodiments, the resultant excision by the one or more gene editors retains in the latently HIV-infected cell an integrated 3′ LTR sequence and at least a portion of a Nef coding sequence (see e.g., FIG. 1A-B). In embodiments, excision by the one or more gene editors retains in the latently HIV-infected cell (e.g., after subsequent non-homologous end joining (NHEJ) or another DNA repair mechanism) the integrated 5′ LTR sequence, TAR Loop sequence, at least a portion of the Gag coding sequence, at least a portion of the Nef coding sequence, the 3′ LTR sequence, and at least one stop codon downstream said Gag coding sequence and upstream said Nef coding sequence and at least one start codon downstream said stop codon and upstream said Nef coding sequence (see e.g., FIG. 1A-B, HIV Proviral Genomic Product).

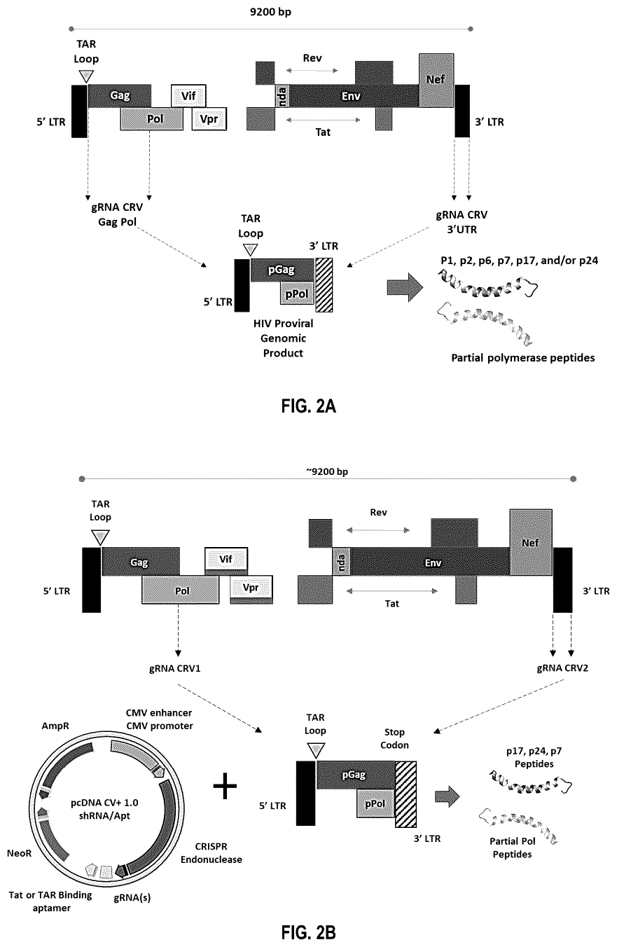

Alternatively, in embodiments, the first gRNA targeted against Gag forms a complex with a site-specific endonuclease (e.g., Cas enzyme) and initiates cleavage of the double stranded DNA of an HIV infected cell with an integrated HIV genome (see e.g., FIG. 2A-B, gRNA CRV Gag Pol); a second gRNA targeted against the 3′ LTR forms a form a complex with a site-specific endonuclease (e.g., Cas enzyme) and initiates cleavage of the double stranded DNA of an HIV infected cell with an integrated HIV genome (see e.g., FIG. 2A-B, gRNA CRV 3′UTR). In embodiments, the resultant excision by the one or more gene editors retains in the latently HIV-infected cell the integrated 5′ LTR sequence, TAR Loop sequence, at least a portion of the Gag coding sequence, and 3′ LTR sequence (see e.g., FIG. 2A-B, HIV Proviral Genomic Product).

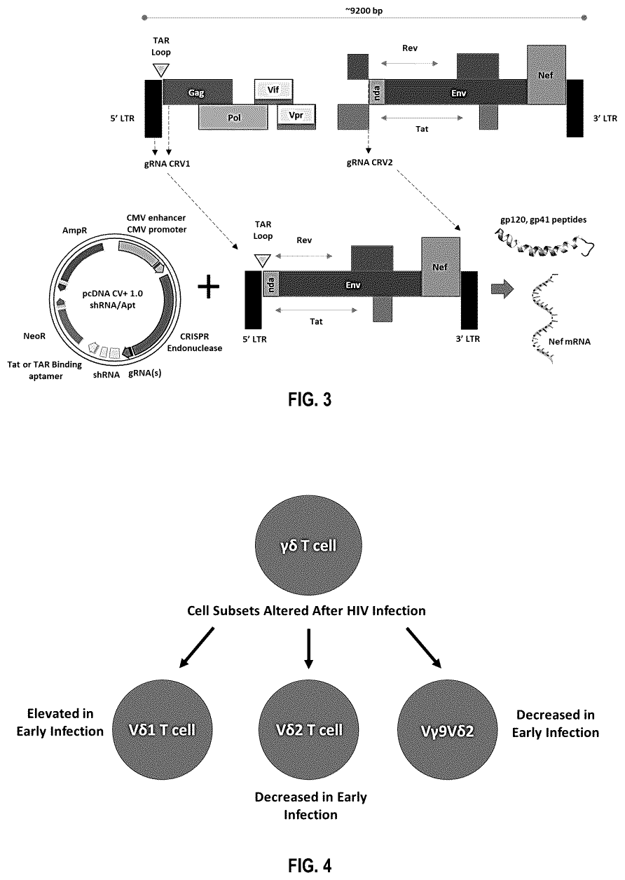

In embodiments, the BioNV-delivered gene editing payload and gRNAs target the 5′ end of the HIV proviral genome (see, e.g., FIG. 3 gRNA CRV1) and 5′ end of the Env proviral sequence (see, e.g., FIG. 3 gRNA CRV2), resulting in excision of the spanning sequence between the gRNA targets. In embodiments, the gRNA can target the TAR loop and/or initiation (Inr) sequence by guiding endonuclease activity within the sequence, or immediately 5′ of the sequence for excision. In embodiments, excision with such a gRNA disrupts an integrated HIV TAR loop sequence. Alternatively, in embodiments, the gRNA can guide endonuclease activity downstream of the TAR loop region so the cell can retain the sequence. In embodiments, excision with such a gRNA retains in the latently HIV-infected cell at least a portion of an integrated 5′ LTR, at least a portion of an HIV Env sequence, and at least a portion of an HIV Nef sequence. Either retained genomic construct, in embodiments, results in retaining at least a portion of Env (i.e., Rev/tat and/or gp120/gp41) and Nef.

In embodiments, the at least two gRNAs comprise: a first gRNA complementary to a first coding sequence and/or a first non-coding sequence; and a second gRNA complementary to a second coding sequence and/or a second non-coding sequence.

In embodiments, the first non-coding sequence is an integrated HIV TAR loop sequence. Targeting such a location by a gene editor, in embodiments, results in disruption of the HIV TAR loop structure. In embodiments, the first non-coding sequence is 5′ (i.e., upstream) of an integrated HIV TAR loop sequence and/or within a 5′ LTR sequence. Targeting such a location by a gene editor, in embodiments, results in excision of the TAR loop structure and Inr sequence.

In embodiments, the first coding sequence is an HIV Gag proviral genomic sequence. In embodiments, an excision site corresponding to the first coding sequence is about or at least about 21 base pairs (bp), about or at least about 24 bp, about or at least about 27 bp, about or at least about 30 bp, about or at least about 33 bp, about or at least about 36 bp, about or at least about 39 bp, about or at least about 50 bp, about or at least about 60 bp, about or at least about 70 bp, about or at least about 80 bp, about or at least about 100 bp, about or at least about 150 bp, about or at least about 200 bp, about or at least about 300 bp, about or at least about 400 bp, or about or at least about 500 bp or more downstream of the Gag protein start codon. In embodiments, the excision site corresponding to the first coding sequence is within the Gag genomic coding sequence sufficient to result in a Gag transcript that is translated into a peptide that can be expressed, intracellularly processed, and presented by an MHC complex.

In embodiments, the second coding sequence and/or a second non-coding sequence is downstream of the first coding sequence and/or the first non-coding sequence and upstream of at least portion of an integrated HIV Env sequence. Targeting such a location by a gene editor, in embodiments, results in retaining an integrated HIV Env sequence (e.g., retaining Rev and Tat), Nef sequence, and the 3′ LTR sequence.

In embodiments, the second coding sequence is an HIV Nef proviral genomic sequence, or wherein the second non-coding sequence is an HIV 3′ LTR genomic sequence. In embodiments, an excision site corresponding to the second coding sequence is about or at least about 21 base pairs (bp), about or at least about 24 bp, about or at least about 27 bp, about or at least about 30 bp, about or at least about 33 bp, about or at least about 36 bp, about or at least about 39 bp, about or at least about 50 bp, about or at least about 60 bp, about or at least about 70 bp, about or at least about 80 bp, about or at least about 100 bp, about or at least about 150 bp, about or at least about 200 bp, about or at least about 300 bp, about or at least about 400 bp, or about or at least about 500 bp or more upstream of the Nef coding sequence stop codon. In embodiments, the excision site corresponding to the second coding sequence is within the Nef genomic coding sequence sufficient to result in a Nef RNA transcript that can be effectively targeted by a small RNA that can bind, silence, or otherwise form a complex with the Nef RNA at the post-transcriptional stage. In embodiments, the excision site corresponding to the second coding sequence is within the Nef genomic coding sequence sufficient to result in a Nef transcript that is translated into a peptide that can be expressed, intracellularly processed, and presented by an MHC complex.

In embodiments, the gRNA is compatible with Cas9 and targets Nef and has a nucleic acid sequence of any one of SEQ ID NOs: 1-20 (e.g., an RNA-version of the DNA sequence, or an RNA sequence complementary to the DNA sequence, as shown in Table 5). In embodiments, the gRNA is compatible with Cas9 and targets Nef and has a nucleic acid sequence of any one of SEQ ID NOs: 1-20 with one or more substitutions, insertions, and/or deletions therein. In embodiments, the gRNA is compatible with Cas9 and targets Nef and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 1-20.

In embodiments, the gRNA is compatible with Cpf1/CasX and targets Nef and has a nucleic acid sequence of any one of SEQ ID NOs: 21-31 (e.g., an RNA-version of the DNA sequence, or an RNA sequence complementary to the DNA sequence, as shown in Table 5). In embodiments, the gRNA is compatible with Cpf1/CasX and targets Nef and has a nucleic acid sequence of any one of SEQ ID NOs: 21-31 with one or more substitutions, insertions, and/or deletions therein. In embodiments, the gRNA is compatible with Cpf1/CasX and targets Nef and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 21-31.

In embodiments, the gRNA is compatible with Cas9 and targets Pol and has a nucleic acid sequence of any one of SEQ ID NOs: 32-51 (e.g., an RNA-version of the DNA sequence, or an RNA sequence complementary to the DNA sequence, as shown in Table 5). In embodiments, the gRNA is compatible with Cas9 and targets Pol and has a nucleic acid sequence of any one of SEQ ID NOs: 32-51 with one or more substitutions, insertions, and/or deletions therein. In embodiments, the gRNA is compatible with Cas9 and targets Pol and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 32-51.

In embodiments, the gRNA is compatible with Cpf1/CasX and targets Pal and has a nucleic acid sequence of any one of SEQ ID NOs: 52-71 (e.g., an RNA-version of the DNA sequence, or an RNA sequence complementary to the DNA sequence, as shown in Table 5). In embodiments, the gRNA is compatible with Cpf1/CasX and targets Pod and has a nucleic acid sequence of any one of SEQ ID NOs: 52-71 with one or more substitutions, insertions, and/or deletions therein. In embodiments, the gRNA is compatible with Cpf1/CasX and targets Pdl and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 52-71.

In embodiments, the gRNA is compatible with Cas9 and targets Rev and has a nucleic acid sequence of any one of SEQ ID NOs: 72-91 (e.g., an RNA-version of the DNA sequence, or an RNA sequence complementary to the DNA sequence, as shown in Table 5). In embodiments, the gRNA is compatible with Cas9 and targets Rev and has a nucleic acid sequence of any one of SEQ ID NOs: 72-91 with one or more substitutions, insertions, and/or deletions therein. In embodiments, the gRNA is compatible with Cas9 and targets Rev and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 72-91.

In embodiments, the gRNA is compatible with Cpf1/CasX and targets Rev and has a nucleic acid sequence of any one of SEQ ID NOs: 92-107 (e.g., an RNA-version of the DNA sequence, or an RNA sequence complementary to the DNA sequence, as shown in Table 5). In embodiments, the gRNA is compatible with Cpf1/CasX and targets Rev and has a nucleic acid sequence of any one of SEQ ID NOs: 92-107 with one or more substitutions, insertions, and/or deletions therein. In embodiments, the gRNA is compatible with Cpf1/CasX and targets Rev and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 92-107.

In embodiments, the gRNA is compatible with Cas9 and targets Gag and has a nucleic acid sequence of any one of SEQ ID NOs: 108-127 (e.g., an RNA-version of the DNA sequence, or an RNA sequence complementary to the DNA sequence, as shown in Table 5). In embodiments, the gRNA is compatible with Cas9 and targets Gag and has a nucleic acid sequence of any one of SEQ ID NOs: 108-127 with one or more substitutions, insertions, and/or deletions therein. In embodiments, the gRNA is compatible with Cas9 and targets Gag and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 108-127.

In embodiments, the gRNA is compatible with Cpf1/CasX and targets Gag and has a nucleic acid sequence of any one of SEQ ID NOs: 128-147 (e.g., an RNA-version of the DNA sequence, or an RNA sequence complementary to the DNA sequence, as shown in Table 5). In embodiments, the gRNA is compatible with Cpf1/CasX and targets Gag and has a nucleic acid sequence of any one of SEQ ID NOs: 128-147 with one or more substitutions, insertions, and/or deletions therein. In embodiments, the gRNA is compatible with Cpf1/CasX and targets Gag and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 128-147.

In embodiments, the gRNA is compatible with Cas9 and targets Tat and has a nucleic acid sequence of any one of SEQ ID NOs: 148-166 (e.g., an RNA-version of the DNA sequence, or an RNA sequence complementary to the DNA sequence, as shown in Table 5). In embodiments, the gRNA is compatible with Cas9 and targets Tat and has a nucleic acid sequence of any one of SEQ ID NOs: 148-166 with one or more substitutions, insertions, and/or deletions therein. In embodiments, the gRNA is compatible with Cas9 and targets Tat and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 148-166.

In embodiments, the gRNA is compatible with Cpf1/CasX and targets Tat and has a nucleic acid sequence of any one of SEQ ID NOs: 167-173 (e.g., an RNA-version of the DNA sequence, or an RNA sequence complementary to the DNA sequence, as shown in Table 5). In embodiments, the gRNA is compatible with Cpf1/CasX and targets Tat and has a nucleic acid sequence of any one of SEQ ID NOs: 167-173 with one or more substitutions, insertions, and/or deletions therein. In embodiments, the gRNA is compatible with Cpf1/CasX and targets Tat and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 167-173.

In embodiments, the gRNA is compatible with Cas9 and targets 5′LTR Sequence of HXB2 and has a nucleic acid sequence of any one of SEQ ID NOs: 174-193 (e.g., an RNA-version of the DNA sequence, or an RNA sequence complementary to the DNA sequence, as shown in Table 5). In embodiments, the gRNA is compatible with Cas9 and targets 5′LTR Sequence of HXB2 and has a nucleic acid sequence of any one of SEQ ID NOs: 174-193 with one or more substitutions, insertions, and/or deletions therein. In embodiments, the gRNA is compatible with Cas9 and targets 5′LTR Sequence of HXB2 and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 174-193.

In embodiments, the gRNA is compatible with Cpf1/CasX and targets 5′LTR Sequence of HXB2 and has a nucleic acid sequence of any one of SEQ ID NOs: 194-210 (e.g., an RNA-version of the DNA sequence, or an RNA sequence complementary to the DNA sequence, as shown in Table 5). In embodiments, the gRNA is compatible with Cpf1/CasX and targets 5′LTR Sequence of HXB2 and has a nucleic acid sequence of any one of SEQ ID NOs: 194-210 with one or more substitutions, insertions, and/or deletions therein. In embodiments, the gRNA is compatible with Cpf1/CasX and targets 5′LTR Sequence of HXB2 and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 194-210.

In embodiments, the gRNA is compatible with Cas9 and targets 3′LTR Sequence of HXB2 and has a nucleic acid sequence of any one of SEQ ID NOs: 211-230 (e.g., an RNA-version of the DNA sequence, or an RNA sequence complementary to the DNA sequence, as shown in Table 5). In embodiments, the gRNA is compatible with Cas9 and targets 3′LTR Sequence of HXB2 and has a nucleic acid sequence of any one of SEQ ID NOs: 211-230 with one or more substitutions, insertions, and/or deletions therein. In embodiments, the gRNA is compatible with Cas9 and targets 3′LTR Sequence of HXB2 and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 211-230.

In embodiments, the gRNA is compatible with Cpf1/CasX and targets 3′LTR Sequence of HXB2 and has a nucleic acid sequence of any one of SEQ ID NOs: 231-247 (e.g., an RNA-version of the DNA sequence, or an RNA sequence complementary to the DNA sequence, as shown in Table 5). In embodiments, the gRNA is compatible with Cpf1/CasX and targets 3LTR Sequence of HXB2 and has a nucleic acid sequence of any one of SEQ ID NOs: 231-247 with one or more substitutions, insertions, and/or deletions therein. In embodiments, the gRNA is compatible with Cpf1/CasX and targets 3′LTR Sequence of HXB2 and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 231-247.

In embodiments, the gRNA is compatible with Cas9 and targets HXB2 TAR (SEQ ID NO: 248) and has a nucleic acid sequence of any one of SEQ ID NOs: 249-254 (e.g., an RNA-version of the DNA sequence, or an RNA sequence complementary to the DNA sequence, as shown in Table 5). In embodiments, the gRNA is compatible with Cas9 and targets HXB2 TAR (SEQ ID NO: 248) and has a nucleic acid sequence of any one of SEQ ID NOs: 249-254 with one or more substitutions, insertions, and/or deletions therein. In embodiments, the gRNA is compatible with Cas9 and targets HXB2 TAR (SEQ ID NO: 248) and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of any one of SEQ ID NOs: 249-254.

In embodiments, the gRNA is compatible with Cpf1/CasX and targets HXB2 TAR (SEQ ID NO: 248) and has a nucleic acid sequence of SEQ ID NO: 255 (e.g., an RNA-version of the DNA sequence, or an RNA sequence complementary to the DNA sequence, as shown in Table 5). In embodiments, the gRNA is compatible with Cpf1/CasX and targets HXB2 TAR (SEQ ID NO: 248) and has a nucleic acid sequence of SEQ ID NO: 255 with one or more substitutions, insertions, and/or deletions therein. In embodiments, the gRNA is compatible with Cpf1/CasX and targets HXB2 TAR (SEQ ID NO: 248) and has a nucleic acid sequence having about or at least about 80%, about or at least about 81%, about or at least about 82%, about or at least about 83%, about or at least about 84%, about or at least about 85%, about or at least about 86%, about or at least about 87%, about or at least about 88%, about or at least about 89%, about or at least about 90%, about or at least about 91%, about or at least about 92%, about or at least about 93%, about or at least about 94%, about or at least about 95%, about or at least about 96%, about or at least about 97%, about or at least about 98%, about or at least about 99% sequence identity of SEQ ID NO: 255.

In embodiments, the small RNA targets an HIV coding sequence. In embodiments, the small RNA targets an HIV coding and/or non-coding sequence and has a nucleic has a nucleic acid sequence of any one of SEQ ID NOs: 1-247 or 249-255 (e.g., an RNA-version of the DNA sequence, or an RNA sequence complementary to the DNA sequence, as shown in Table 5), including nucleic acid sequences having one or more substitutions, insertions, and/or deletions therein.

In embodiments, the small RNA targets an HIV Gag coding sequence or an HIV Nef coding sequence. In embodiments, the HIV Nef coding sequence is a portion of a messenger RNA encoding a Nef protein.

In embodiments, the small RNA is configured to interact with an RNA-induced silencing complex (RISC) in the latently HIV-infected cell. In embodiments, Argonaute-bound miRNAs can exist mainly in high molecular weight RNA-induced silencing complexes (HMW-RISC) associated with target mRNA. It has been shown that most adult tissues contain reservoirs of miRNAs in low molecular might RISC (LMW-RISC) not bound to mRNA, and that the majority of individual miRNAs for example in primary T cells are enriched in LMW-RISC, for example as described in La Rocca G, et al. “in vivo, Argonaute-bound microRNAs exist predominantly in a reservoir of low molecular weight complexes not associated with mRNA,” Proc Natl Acad Sci USA. Vol. 112, No. 3, 2015: pp. 767-72, the entire contents of which are hereby incorporated by reference in its entirety.

In embodiments, during T cell activation (e.g., during T cell-MHC interaction due to the expression and presentation of the one or more HIV antigens as a result of the methods herein), signal transduction through the phosphoinositide-3 kinase-RAC-alpha serine/threonine-protein kinase-mechanistic target of rapamycin pathway can increase the assembly of miRNAs into HMW-RISC, which initiates a positive feedback loop of enhanced expression of the glycine-tryptophan protein of 182 kDa, an essential component of HMW-RISC, and improves the ability of miRNAs to repress partially complementary reporters, even when expression of targeting miRNAs does not increase (La Rocca, et al. 2015).

In embodiments, the interaction with the RISC complex induces silencing of the HIV Nef RNA sequence. In embodiments, the interaction with the RISC complex induces amplification of the small RNA. In embodiments, the small RNA is configured to be trafficked from the latently HIV-infected cell to at least a second HIV-infected cell. In embodiments, the small RNA functions to prevent and/or reverse Nef-mediated multiple histocompatibility complex (MHC) sequestration in the latently HIV-infected cell and/or the second HIV-infected cell. In embodiments, the trafficking is through a SIDT-1/2 cell surface protein complex.

In embodiments, the small RNA is one or more of a tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), and small non-coding RNA. In embodiments, the small RNA is a RNAi. In embodiments, the small RNA is a shRNA.

In embodiments, the small RNA ranges in size between about 16 nucleotides (or mer) to about 100 nucleotides. In embodiments, the small RNA is at least about 16 mer, at least about 17 mer, at least about 18 mer, at least about 19 mer, at least about 20 mer, at least about 21 mer, at least about 22 mer, at least about 23 mer, at least about 24 mer, at least about 25 mer, at least about 26 mer, at least about 27 mer, at least about 28 mer, at least about 29 mer, at least about 30 mer, at least about 35 mer, at least about 40 mer, at least about 45 mer, at least about 50 mer, at least about 55 mer, at least about 60 mer, at least about 65 mer, at least about 75 mer, at least about 80 mer, at least about 85 mer, at least about 90 mer, at least about 95 mer, or at least about 100 mer or more in length.

BioNVs, in embodiments, encapsulate one or more additional nucleic acids. In embodiments, the one or more additional nucleic acids encodes one or more gene editing payloads (e.g., a CRISPR endonuclease and/or site-directed nuclease). In embodiments, the one or more additional nucleic acids encodes one or more gRNA sequences. In embodiments, the one or more additional nucleic acids encodes the one or more additional nucleic acids encodes one or more small RNAs that targets an HIV sequence. In embodiments, the one or more additional nucleic acids comprise one or more low-level expression constitutively active promoters, tissue-specific promoters, cell-specific promoters, and/or suicide promoters.

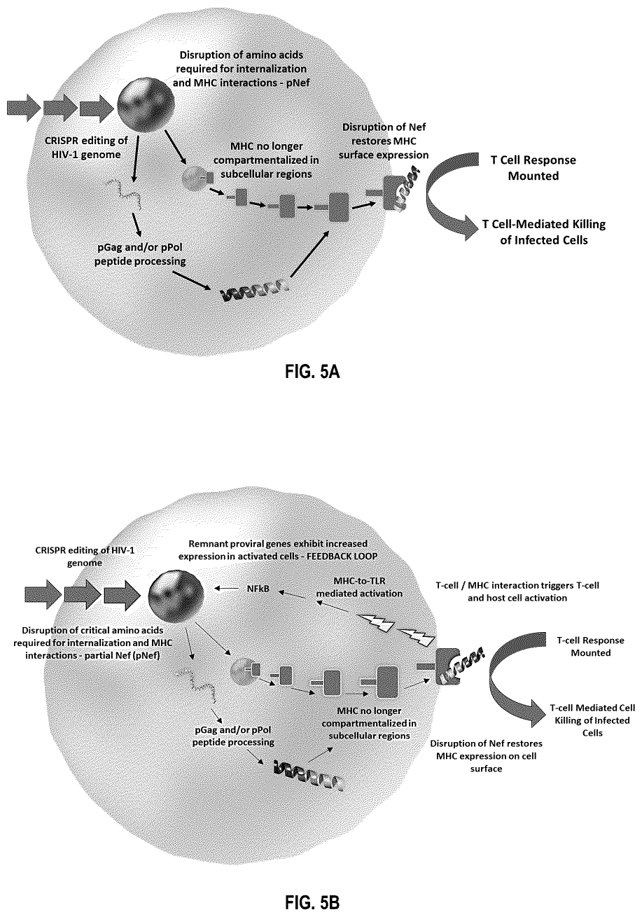

In embodiments, the BioNV encapsulates one or more additional nucleic acids that encodes an HIV Tat sequence. Tat expression, in embodiments, is used to bind the TAR loop structure in the 5′ region of the retained proviral sequence to enable transcription of proviral sequences. In embodiments, NFκB expression in the infected cell results in a positive feedback loop that drives expression from upstream of the TAR loop (e.g., as shown in FIG. 5B). In embodiments, the Tat construct encoded in the additional nucleic acid is mutated to selectively bind and activate the TAR loop with minimized toxicity in comparison to a wild-type Tat construct. In embodiments, the Tat construct is operably linked to an expression control element such that Tat expression is confined only to cells that are infected by HIV and/or only cells that uptake the BioNV. In embodiments, Tat constructs are operably linked to low-level expression constitutively active promoters, tissue-specific promoters, cell-specific promoters, and/or suicide promoters.

In embodiments, the BioNV encapsulates one or more additional nucleic acids that encodes one or more aptamers configured to bind an HIV TAR sequence. In embodiments, aptamers are designed to be an “activating aptamer” which binds the HIV TAR loop region and enable transcription of retained downstream sequences.

In embodiments, the BioNV can deliver a plasmid (e.g., containing the mechanistic properties to direct and pass it into the nucleus in a cell) encoding one or more gene editing payloads (e.g., a CRISPR endonuclease), gRNA(s), shRNA, a Tat expression cassette, and/or a TAR loop binding aptamer (e.g., as shown in FIGS. 1B, 2B, and 3). In embodiments, expression of Env and Nef can be driven by the TAR loop and initiator sequence (Inr) via Tat expression from a promoter on the same plasmid as the gene editing payload and/or by expression of an activating TAR loop-binding aptamer. Alternatively, and without wishing to be bound by theory, in embodiments, the TAR loop region is disrupted and low levels of background expression of retained HIV proviral genes occurs without an intact 5′ TAR loop and Inr sequences.

BioNVs, in embodiments, encapsulate one or more latency reversal agents (LRAs). LRAs, in embodiments, are compounds that function to initiate a reversal from latency (i.e., viral dormancy) by coercing infected cells to express viral antigens, no longer allowing viral immune evasion. LRAs, in embodiments, include PKC agonists (e.g., Prostratin Bryostatin-1 Ingenols: Ingenol-B, Ingenol 3, 20-dibenzoate (Ingenol-db), Ingenol-3-angelate (Ingenol mebutate, PEP005); MAPK agonists (e.g., Procyanidin trimer Cl); CCR5 antagonists (e.g., Maraviroc); Tat vaccine components (e.g., Tat Oyi vaccine, Tat-R5M4 protein); SMAC mimetics (e.g., SBI-0637142, Birinapant); Inducers of P-TEFb release (e.g., BETis: JQ1, I-BET, 1-BET151, OTX015, UMB-136, MMQO, CPI-203, RVX-208, PFI-1, BI-2536, and BI6727, HMBA); Activators of Akt Pathway (e.g., Disulfiram); Beonzotriazole derivatives (e.g., 1-hydroxybenzotraizol (HOBt)); Epigenetic modifiers (e.g., HDACis: TSA, trapoxin, SAHA, romidepsin, panobinostat, entinostat, givinostat, valproic acid, MRK-1/11, AR, fimepinostat, chidamide, HMTis: chaetocin, EPZ-6438, GSK-343, DZNEP, BIX-01294, UNC-0638); Immunomodulatory LRAs (e.g., TLR agonists: TLR2 (Pam3CSK4), TLR7 (GS-9620), TLR8, TLR9 (MGN 1703) agonists, IL-15 agonists (ALT-803), immune checkpoint inhibitors: anti-PD-1 (nivolumab, pembrolizumab), anti-CTLA-4 (ipilimumab). In embodiments, methods of treatment herein include administering LRAs (e.g., as a co-administration with a BioNV and/or encapsulated in a BioNV), for example as described in (AIT-AMMAR, et al., “Current Status of Latency Reversing Agents Facing the Heterogeneity of HIV-1 Cellular and Tissue Reservoirs,” Front Microbiol, Vol. 10, No. 3060, 2020: pp. 1-23).

BioNVs, in embodiments, encapsulate one or more antiretroviral therapies. In embodiments, the antiretroviral therapy includes a non-nucleoside reverse transcriptase inhibitor (NNRTI), optionally selected from efavirenz (SUSTIVA), rilpivirine (EDURANT) and doravirine (PIFELTRO). In embodiments, the antiretroviral therapy includes a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY). In embodiments, the antiretroviral therapy includes a protease inhibitor (PI), optionally selected from atazanavir (REYATAZ), darunavir (PREZISTA), and lopinavir/ritonavir (KALETRA). In embodiments, the antiretroviral therapy includes an integrase inhibitor, optionally selected from bictegravir sodium/emtricitabine/tenofovir alafenamide fumar (BIKTARVY), raltegravir (ISENTRESS), and dolutegravir (TIVICAY). In embodiments, the antiretroviral therapy includes an entry or fusion inhibitor, optionally selected from enfuvirtide (FUZEON) and maraviroc (SELZENTRY).

BioNVs are, in embodiments, about 10-1200 nm in diameter. In embodiments, BioNVs are about 10 nm in size, about 20 nm in size, about 30 nm in size, about 40 nm in size, about 50 nm in size, about 60 nm in size, about 70 nm in size, about 80 nm in size, about 90 nm in size, about 100 nm in size, about 120 nm in size, about 140 nm in size, about 160 nm in size, about 180 nm in size, about 200 nm in size, about 300 nm in size, about 400 nm in size, about 500 nm in size, about 600 nm in size, about 700 nm in size, about 800 nm in size, about 900 nm in size, about 1000 nm in size, about 1100 nm in size, or about 1200 nm in size. In embodiments, BioNVs range in size from about 10 nm to 20 nm in size, about 20 nm to 30 nm in size, about 30 nm to 40 nm in size, about 40 nm to 50 nm in size, about 50 nm to 60 nm in size, about 60 nm to 70 nm in size, about 70 nm to 80 nm in size, about 80 nm to 90 nm in size, about 90 nm to 100 nm in size, about 10 nm to 100 nm in size, about 100 nm to 200 nm in size, about 200 nm to 400 nm in size, about 400 nm to 600 nm in size, about 600 nm to 800 nm in size, about 800 nm to 1000 nm in size, about 1000 nm to 1200 nm in size, or about 10 nm to 1200 nm in size.

In embodiments, the BioNV is stored at about −80° C. or suitable for storage at about −80° C. In embodiments, the BioNV is lyophilized (e.g., for reconstitution in buffer) or suitable for lyophilization. In embodiments, BioNVs can be stable at about ambient temperature, at about −20° C., at about 4° C., at about 25° C., or at about 37° C. for at least about 1 hour, at least about 2 hours, at least about 4 hours, at least about 6 hours, at least about 12 hours, at least about 24 hours, at least about 2 days, at least about one week, or at least about one month or longer.

### Methods of Treating, Preventing, or Ameliorating HIV

In aspects, the present disclosure includes methods for treating, preventing, and/or ameliorating an HIV infection in a subject. In embodiments, the method includes treating an HIV infection by administering to a subject in need thereof a BioNV, as described herein.

In aspects, the present disclosure includes methods for treating, preventing, and/or ameliorating an HIV infection in a subject by administering to the subject a BioNV that has a targeting agent targeted against at least a first cell surface marker of a latently HIV-infected cell, and is configured to deliver: a) a gene editing payload comprising at least two gRNAs, including i) a first gRNA targeted against an HIV Gag proviral genomic sequence, and ii) a second gRNA targeted against an HIV proviral 3′ LTR genomic sequence, wherein the gene editing payload is capable of excising the HIV proviral genomic sequence between the HIV Gag proviral genomic sequence and the HIV 3′ LTR proviral genomic sequence, and wherein an HIV 5′ LTR sequence, TAR Loop sequence, at least a portion of the HIV Gag proviral genomic sequence, and at least a portion of the HIV 3′ LTR proviral genomic sequence is not excised from the latently HIV-infected cell (see e.g., as shown in FIGS. 2A and 2B).

In aspects, the present disclosure includes methods for treating, preventing, and/or ameliorating an HIV infection in a subject by administering to the subject a BioNV that has a targeting agent targeted against at least a first cell surface marker of a latently HIV-infected cell, and is configured to deliver: a) a gene editing payload comprising at least two gRNAs, including: i) a first gRNA targeted against an HIV Gag proviral genomic sequence, and ii) a second gRNA targeted against an HIV proviral Nef genomic sequence, wherein the gene editing payload is capable of excising the HIV proviral genomic sequence between the HIV Gag proviral genomic sequence and the HIV Nef proviral genomic sequence, and wherein an HIV 5′ LTR sequence, TAR Loop sequence, at least a portion of the HIV Gag proviral genomic sequence, and at least a portion of the HIV Nef proviral genomic sequence is not excised from the latently HIV-infected cell, and b) an RNAi that is targeted against the mRNA sequence of the HIV Nef proviral genomic sequence that is not excised (see e.g., as shown in FIGS. 1A and 1B).

In aspects, the present disclosure includes methods for treating, preventing, and/or ameliorating an HIV infection in a subject by administering to the subject a BioNV that is targeted against at least a first cell surface marker of a latently HIV-infected cell, and is configured to deliver: a) a gene editing payload comprising at least two gRNAs, wherein the at least two gRNAs comprise: i) a first gRNA targeted against an HIV proviral genomic sequence within the TAR loop, 5′ of the TAR loop, e.g. immediately 5′ of the TAR loop; and ii) a second gRNA targeted against upstream of at least a portion of an HIV Env proviral genomic sequence, wherein the gene editing payload is capable of excising the HIV proviral genomic sequence between the first gRNA target sequence and the second gRNA target sequence, and wherein at least a portion of a 5′ end of the HIV proviral genomic sequence and a portion of the HIV Env proviral genomic sequence is not excised from the latently HIV-infected cell; and b) an interference RNA (RNAi), wherein the RNAi is targeted against a messenger RNA sequence complementary to a portion of an HIV Nef proviral genomic sequence that is not excised, wherein the latently HIV-infected cell expresses a partial Nef RNA sequence (pNef) from the portion of the HIV Nef proviral genomic sequence which reduces Nef-mediated MHC complex sequestration in the latently HIV-infected cell, wherein the latently HIV-infected cell expresses a partial gp120 and/or gp41 peptide from the portion of the HIV Env proviral genomic sequence that is not excised, which is recognized by the MHC complex and results in T-cell stimulation against the latently HIV-infected cell, and wherein the pNef and gp120/gp41 expression in the latently HIV-infected cell results in: a) processing and presentation of gp120/gp41 peptides via an MHC; b) cell-to-cell spread of the RNAi via the SIDT-1/2 complex; and c) T-cell mediated cytotoxicity against one or more latently HIV-infected cells (see e.g., as shown in FIG. 3).

In aspects, the present disclosure includes methods for treating, preventing, and/or ameliorating an HIV infection in a subject by administering to the subject a BioNV that is targeted against at least a first cell surface marker of a latently HIV-infected cell and is configured to deliver: a) a gene editing payload comprising at least two gRNAs, wherein the at least two gRNAs comprise: i) a first gRNA targeted against an HIV Gag proviral genomic sequence; and ii) a second gRNA targeted against upstream of at least a portion of an HIV Env proviral genomic sequence, wherein the gene editing payload is capable of excising the HIV proviral genomic sequence between the first gRNA target sequence and the second gRNA target sequence, and wherein at least a portion of an HIV TAR loop and 5′ LTR proviral genomic sequence and at least a portion of the HIV Env proviral genomic sequence is not excised from the latently HIV-infected cell; and b) an interference RNA (RNAi), wherein the RNAi is targeted against a messenger RNA sequence complementary to a portion of an HIV Nef proviral genomic sequence that is not excised, wherein the latently HIV-infected cell expresses a partial Nef RNA sequence (pNef) from the portion of the HIV Nef proviral genomic sequence which reduces Nef-mediated MHC complex sequestration in the latently HIV-infected cell, wherein the latently HIV-infected cell expresses a partial gp120 and/or gp41 peptide from the portion of the HIV Env proviral genomic sequence that is not excised, which is recognized by the MHC complex and results in T-cell stimulation against the latently HIV-infected cell, and wherein the pNef and gp120/gp41 expression in the latently HIV-infected cell results in: a) processing and presentation of gp120/gp41 peptides via an MHC; b) cell-to-cell spread of the RNAi via the SIDT-1/2 complex; and c) T-cell mediated cytotoxicity against one or more latently HIV-infected cells (see e.g., as shown in FIG. 3).

Methods of treating, preventing, and/or ameliorating an HIV infection described herein, in embodiments, eliminate the HIV Nef gene and other critical genes, which permanently disables the HIV viral replication cycle in both lytic cycle cells and latently infected cells. By eliminating Nef, in embodiments, HIV can no longer sequester MHC receptors, thus disabling its immune evasion. In embodiments, methods of treating, preventing, and/or ameliorating an HIV infection herein leave intact one or more proviral genes. In embodiments, methods of treating, preventing, and/or ameliorating an HIV infection herein leave intact one or more protein-encoding proviral genes. Upon expression, in embodiments, these proteins are processed into peptides by cellular machinery and exposed on the newly replenished MHC receptors for T cell engagement. Upon T cell engagement with the MHC, in embodiments, T cell repertoires can build immunological memory to recognize other HIV-infected cells.

Methods of treating, preventing, and/or ameliorating an HIV infection described herein, in embodiments, improves on methods that eliminate the entire HIV genome because eliminating or disabling the entire proviral genome does not enable expression of viral peptides for MHC exposure.

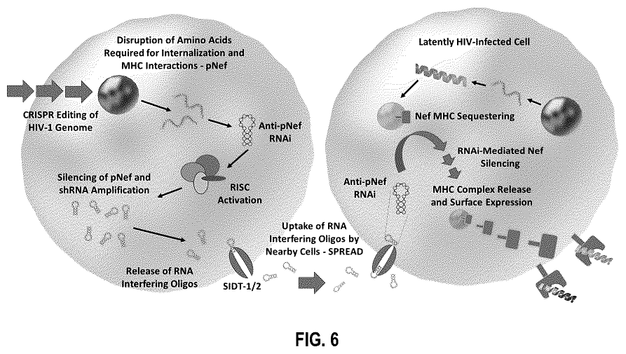

In embodiments, the present disclosure includes methods for treating, preventing, or ameliorating an HIV infection by administering a BioNV which results in the latently HIV-infected cell expressing: a partial Gag protein (pGag) from a portion of the portion of the HIV Gag proviral genomic sequence that is not excised, which is recognized by a MHC complex and results in T-cell stimulation against the latently HIV-infected cell (see e.g., as shown in FIGS. 1A-1B, 2A-2B, 5A-5B) and a partial Nef RNA sequence (pNef) from a portion of the HIV Nef proviral genomic sequence that is not excised (see e.g., as shown in FIGS. 1A-1B and 3), which reduces Nef-mediated MHC complex sequestration in the latently HIV-infected cell, and wherein expression of pNef and pGag in the latently HIV-infected cell results in: a) processing and presentation of the pGag via an MHC, b) cell-to-cell spread of the RNAi via the SIDT-1/2 complex, and c) T-cell mediated cytotoxicity against one or more latently HIV-infected cells (see e.g., as shown in FIGS. 5A-5B and 6).

In embodiments, the gene editor targets HIV Negative Regulatory Factor (Nef). In embodiments, the HIV Nef is chromosomally integrated. Nef is an HIV-1 protein that is expressed from the 3′ region of the integrated genome near the 3′ LTR. Nef is a small 27-35 kDa myristoylated protein that is involved in the downregulation of MHC class I transmembrane antigen presenting complexes by the compartmentalization into lysosomal organelles (Lenassi, et al. “HIV Nef is secreted in exosomes and triggers apoptosis in bystander CD4+ T cells.” Traffic. Vol. 11, No. 1, 2010: pp. 110-122), (Dirk, et al. “HIV-1 Nef sequesters MHC-1 intracellularly by targeting early stages of endocytosis and recycling.” Vol. 6, No. 37021, 2016: pp. 1-13). Mutations in Nef have been found to restore MHC expression and consequent T-cell responses.

In embodiments, methods herein do not excise the entire HIV-1 genome; rather the methods disrupt the HIV genome at two designated regions (e.g., one at the Gag region and one at the Nef region or 3′ LTR region), which results in a residual, benign, and non-infective partial HIV genome that expresses partial and low-toxicity-associated viral transcripts/peptides. In embodiments, these partial viral transcripts/peptides are capable of being processed into the cleft for exposure to and stimulation of T-cell responses (see e.g., as shown in FIG. 4). In embodiments, this approach results in: 1) superior gene editor delivery using the BioNV compared to AAV and LNP approaches; 2) partial disruption of the HIV genome such that it is no longer infectious; 3) a remnant of the HIV genome that produces partial HIV transcripts/peptides for MHC presentation; 4) partial disruption of the Nef gene that prevents and/or reduces Nef-mediated MHC compartmentalization; 5) T-cell stimulation through the MHC presentation of the residual peptides (e.g., pGag); and 6) elimination of HIV-infected cells.

In embodiments, methods herein include delivery of a gene editing payload for excision of the HIV genome and introducing mutations in the CCR5 delta region of the host cell.

In embodiments, methods include excision of an internal section of the HIV genome (see e.g., as shown in FIGS. 1A-1B, 2A-2B, and 3). In embodiments, the excision leaves a gp17 Gag N-terminal portion and a Nef C-terminal portion to generate MHC-loadable immunogenic peptides (Boucau and Gall. “Antigen processing and presentation in HIV infection.” Mol. Immunol. Vol. 113, 2019: pp. 67-74). In embodiments, these peptides (Gag peptides, e.g., HIV-1 capsid protein for HLA-E, etc.) are selected because they have been shown to induce strong affinity for MHC presentation among HIV peptides, as well as their moderate cellular toxicity in comparison to other HIV proteins (e.g., Pol, Tat, Env) (Mattson, et al. “Cell Death in HIV dementia.” Cell Death and Differentiation. Vol. 12, 2005: pp. 893-904), (Davis, et al. “A Conserved HIV-1-Derived Peptide Presented by HLA-E Renders Infected T-cells Highly Susceptible to Attack by NKG2A/CD94-bearing Natural Killer Cells. PLOS Pathogens. Vol. 12, No. 2, 2016: pp. 1-22), (Zeinolabediny, et al. “HIV-1 matrix protein p17 misfolding forms toxic amyloidogenic assemblies that induce neurocognitive disorders.” Sci. Rep. Vol. 7, No. 10313, 2017: pp. 1-18). Without wishing to be bound by theory, in embodiments, without the Gag peptides the T-cell response may not be as robust (strong and maintained) if they are excised. In embodiments, the MHC-loadable peptides stimulate a localized immune response while the functional proteins necessary for HIV replication are eliminated.

In embodiments, methods include delivery of an RNAi (e.g., a 21-27 mer hairpin RNA) that targets pNef (partial mRNA), which rescues Nef-mediated MHC compartmentalization. Without wishing to be bound by theory, in embodiments, the RNAi can spread cell-to-cell via a pore protein complex in T cells (SIDT-1/2), where the pore opens up during MHC engagement and subsequent T cell activation. In embodiments, at least 2 or more co-treatments, at least 3 or more co-treatments, or at least 4 or more co-treatments are delivered as a single payload.

Methods of treating, preventing, and/or ameliorating an HIV infection described herein, in embodiments, use gene editing to eliminate the Gag, Pol, Vif and Vpr genes of the HIV provirus, thereby permanently blocking its replication cycle. In embodiments, methods herein leave intact, or at least partially intact, the HIV Tat, Rev, Env and Nef genes. In embodiments, Nef is silenced with an shRNA that is expressed from the same plasmid (e.g., containing the mechanistic properties to direct and pass it into the nucleus in a cell) as the gene editor and gRNA(s). In embodiments, the shRNA activates the RISC silencing pathway, leading to the silencing of Nef and the subsequent release of sequestered MHC receptors. Once the receptors are expressed on the surface of the infected cell, in embodiments, T cell engagement trains the immune system to recognize other infected cells. In embodiments, upon TCR engagement with the MHC, the cells become activated against HIV-infected cells providing longer-term immunity. Upon activation, in embodiments, the SIDT1/T2 RNA interfering transfer pores become active to spread the Nef-silencing signal to other immune cells.

Methods of treating, preventing, and/or ameliorating an HIV infection described herein, in embodiments, use multi-optional dosing capable AAVs (e.g., AAV DJ/DJ8 system). In embodiments, AAVs (e.g., AAV DJ/DJ8 systems) deliver a nucleic acid payload that encodes one or more of: i) one or more site-directed endonucleases, CRISPR/Cas endonucleases, or another gRNA-compatible nuclease, ii) two or more gRNAs, iii) one or more small RNAs, iv) one or more Tat expression elements, v) one or more TAR loop binding aptamers, and vi) gene expression control element (e.g., as shown in FIGS. 1B, 2B, and 3). AAV delivery systems, in embodiments, can be used to delivery nucleic acid payloads, as described herein, to specific cell subsets and/or tissues in eliminating latent HIV.

In embodiments, methods herein operate by a mechanism where the infected cells are not rescued from HIV infection by genome excision, but rather they are killed by T cell-mediated cytotoxicity. Cataloguing of cellular “generals,” or deep memory T-cells (also called γδ T-cells), in HIV patients reveals that T cell reservoir populations are altered (see e.g., FIG. 4), with some populations being severely diminished (Pauza, et al. “γδ T cells in HIV disease: past, present, and future.” Front. Immunol. Vol. 5, No. 687, 2015:1-11). The receptor ligand repertoire that is presented on the γδ T cell subset, Vδ2 T cells, and subsets that present via the Vγ9Vδ2 receptor, are eliminated in HIV infection, especially those of Gag-specific CD4+ T cells (Hebbeler, et al. “Failure to Restore the Vγ2-Jγ1.2 Repertoire in HIV-infected Men Receiving Highly Active Antiretroviral Therapy (HAART).” Clin. Immunol. Vol. 128, No. 3, 2008: pp. 349-357). The population of “General” cells in T cell repertoires recovers in patients that have been on ART for greater than 7 years, but the newly recovered cells are not derived from the original pre-infection repertoire (Chaudhry, et al. “The γδ T-cell repertoire is reconstituted in HIV patients after prolonged antiretroviral therapy.” AIDS. Vol. 27, No. 10, 2013: pp. 1557-1562). Therefore, any HIV-specific T cell memory is lost. Without wishing to be bound by theory, in embodiments, any new clonal populations of Vγ9Vδ2 receptor cell subsets will be largely uninfected due to expansion under the persistence of ART, and therefore will remain unaffected by the approaches described herein. Further, without wishing to be bound by theory, in embodiments, methods described herein eliminate dysfunctional CD4+ T cells outside of the Vγ9Vδ2 receptor subsets that are epigenetically damaged by HIV protein expression perseverance.

In embodiments, treatment methods are based on the subject's time spent on ART and/or the subjects detectable T cell populations. For example, in embodiments, subjects with greater than 7 years on ART who exhibit recovered Vγ9Vδ2 T cell counts, and subjects with less than 7 years on ART without recovered Vγ9Vδ2 T cel counts receive different BioNV treatments. As shown in FIG. 4, Vδ1 T cells are elevated in early HIV infection (and may have antiviral functions through killing of infected cells using the NKp30 or NKG2C recognition receptors), whereas Vδ2 T cells are decreased. Although, a small fraction of HIV patients exhibit normal Vδ2 T cell level and/or function; these are elite controllers or natural virus suppressors, defined as HIV patients with undetectable viremia and no history of ART (except for occasional flair-up periods of detectable viremia). Among these patients (making up approximately 0.5% of all persons with HIV infection), Vδ2 cell levels are equivalent to age, gender, and race-matched uninfected controls, but there are significant differences in the Vγ9 chain repertoire. The lingering defect in certain Vδ2 T cell populations and the linked failure to co-stimulate NK cells for DC killing likely increases the risk for chronic activation/inflammation as potent antigen-presenting cells accumulate in the absence of normal control mechanisms.

Patients with more than 7 years of ART show extensive reconstitution of the TCR cell repertoire (based on Vγ9 chain sequencing) and exhibit a population of Vγ9JP chains having nearly the complexity found in healthy, HIV-negative controls (Chaudhry et al., 2013). Patients undergoing treatment generally show nadir CD4 counts below 100 cells/mm³ and are not expected to have circulating Vγ9JP cells (Chaudhry et al., 2013). Therefore, without wishing to be bound by theory, in embodiments, BioNVs derived from CAR-NK cells (or derived from γδ T cells) can replace the lost γδ T cell costimulation of NK cells in HIV infected individuals and aid in immune system boosting during treatment.

In embodiments, the HIV infection is a latent infection. In embodiments, the HIV can be HIV-1 or HIV-2. In embodiments, HIV-1 can refer to one of Group M, Group N, Group O, Group P, or any combination thereof. In embodiments, the HIV-1 is Group M and a subtype selected from subtype A, B, C, D, F, G, H, I, J, K, and L.

In embodiments, administering the BioNV results in excision by the gene editing payload of the HIV proviral genome between the target sequences of the at least two gRNAs (see e.g., FIGS. 1A-1B, 2A-2B, and 3). In embodiments, the excision by the one or more gene editors retains in the latently HIV-infected cell an integrated 5′ LTR sequence, TAR Loop sequence, and an at least a portion of a Gag coding sequence. In embodiments, the excision by the one or more gene editors retains in the latently HIV-infected cell an integrated 3′ LTR sequence and at least a portion of a Nef coding sequence. In embodiments, the excision by the one or more gene editors retains the integrated 5′ LTR sequence, TAR Loop sequence, at least a portion of the Gag coding sequence, at least a portion of the Nef coding sequence, the 3′ LTR sequence, and at least one stop codon downstream the Gag coding sequence and upstream the Nef coding sequence and at least one start codon downstream the stop codon and upstream the Nef coding sequence. In embodiments, the excision by the one or more gene editors retains the integrated 5′ LTR sequence, TAR Loop sequence, at least a portion of the Gag coding sequence, and 3′ LTR sequence.

In embodiments, administering the BioNV results in disruption of an integrated HIV TAR loop sequence. Without wishing to be bound by theory, in non-limiting embodiments, targeting sequences within the TAR loop, or 5′ of the TAR loop, e.g., immediately 5′ of the TAR loop, result in eliminating this structure while retaining low levels of activation that still allow expression of the downstream proviral sequences (i.e., viral peptides) necessary for MHC presentation (e.g., once Nef has been either eliminated (i.e., via excision) or suppressed (i.e., via shRNA)).

In embodiments, the excision retains in the latently HIV-infected cell at least a portion of an integrated 5′ LTR sequence, at least a portion of an HIV Env sequence, and at least a portion of an HIV Nef sequence.

Methods herein that maintain the TAR loop structure, in embodiments, include one or more of a latency reversal agent (LRA), expression of Tat, or a TAR activating aptamer. The TAR loop structure, in embodiments, is maintained to assist with expression of proviral HIV genes that are not excised. In embodiments, one or more of a LRA, Tat expression, or a TAR aptamer functions to activate the TAR loop and Inr sequence to express one or more HIV proviral sequences and/or viral peptides.

Methods herein that maintain the TAR loop structure, in embodiments, can allow for canonical NFκB-induced HIV proviral expression (e.g., as shown in FIG. 5B). Without wishing to be bound by theory, in embodiments, NFκB signaling due to MHC-to-TLR mediated activation in HIV-infected cells that have been targeted by the BioNV can result in a positive feedback loop where the remnant proviral genes that are not excised are expressed.

Expression of Tat in the HIV-infected cell, in embodiments, can be due, in whole or in part, to retaining the Tat sequence after excision (e.g., as shown in FIG. 3) and/or due, in whole or in part, to expression of Tat from a nucleic acid payload encapsulated within the BioNV. The Tat sequence encapsulated in the BioNV, in embodiments, is mutated for reduced toxicity when expressed in the cell. In embodiments, the Tat sequence is operably linked to a regulatable gene expression element, for example and without limitation, a cell-specific promoter, tissue-specific promoter, suicide promoter, etc. In embodiments, Tat expression is limited to HIV-infected cells. Without wishing to be bound by theory, Tat expression, in embodiments, is tightly confined to BioNV-targeted cells as a TAR loop activating measure to ensure expression of one or more HIV proviral sequences that are not excised from the cell.

A TAR “activating” aptamer, in embodiments, is a nucleic acid that does not repress transcriptional activation from the TAR loop, but rather, in whole or in part, functions to bind the TAR loop structure to enable transcription of one or more HIV proviral genes. In embodiments, one or more TAR activating aptamers is encoded in a nucleic acid payload encapsulated in the BioNV. In embodiments, the one or more TAR loop aptamer sequences is operably linked to a regulatable gene expression element, for example and without limitation, a cell-specific promoter, tissue-specific promoter, suicide promoter, etc. In embodiments, aptamer expression is limited to HIV-infected cells. Without wishing to be bound by theory, aptamer expression, in embodiments, is tightly confined to BioNV-targeted cells as a TAR loop activating measure to ensure expression of one or more HIV proviral sequences that are not excised from the cell.

In embodiments, administering the BioNV results in the expression of one or more HIV proteins or one or more portions thereof in an HIV-infected cell (see e.g., FIG. 5A-58). The one or more HIV proteins, in embodiments, is p1, p2, p6, p7, p17, p24, gp41, gp120, and/or an HIV polymerase. In embodiments, the one or more HIV proteins is processed and presented via a multiple histocompatibility complex (MHC) in the latently HIV-infected cell.

Methods herein, in embodiments, utilize a Nef-decoupling gene editing strategy. In embodiments, the BioNV delivers a small RNA. Administering the BioNV, in embodiments, results in expression of a partial Nef RNA (pNef). In embodiments, the pNef transcript is targeted by the small RNA, as shown, without limitation, in FIG. 6. In embodiments, conserved regions of Nef are targeted, minimizing evasion due to mutations in the Nef mRNA sequence. Alternatively, in embodiments, variant-specific Nef mutant sequences are targeted, with individual patient subpopulation specificity.

Within the context of HIV infection, Nef functions to compartmentalize MHCs in subcellular regions. In embodiments, disruption of amino acids required for internalization and MHC interactions in Nef will result in the prevention of MHC compartmentalization in subcellular regions, for example and without limitation, as described in (ALI, et al. “A Novel HIV-1 Nef Mutation in a Primary Pediatric Isolate Impairs MHC-Class I Downregulation and Cytopathicity.” AIDS Res. Hum. Retrovir. Vol. 36, No. 2, 2020: pp. 122-130) and (JOHNSON, et al., “A Highly Conserved Residue in HIV-1 Nef Alpha Helix 2 Modulates Protein Expression,” mSphere, Vol. 1, No. 6, 2016: pp. 1-17). Thus, in embodiments, the small RNA functions to prevent and/or reverse Nef-mediated MHC sequestration in the latently HIV-infected cell (i.e., disruption of Nef restores MHC expression on the cell surface). In embodiments, without wishing to be bound by theory, the inhibition of Nef-mediated MHC sequestration and the expression of HIV antigens will enable T cell responses to be mounted for building immunological memory in the subject, as well as continued T cell-mediated killing of HIV infected cells and reduction of persistence (Duette, et al. “The HIV-1 proviral landscape reveals that Nef contribute to HIV-1 persistence in effector memory CD4+ T cells.” J. Clin. Investig. Vol. 132, No. 7, 2022: pp. 1-17).

HIV-1 Nef protein turns over quickly as observed by pulse chase (JOHNSON et al., 2016). In embodiments, small RNAs target conserved regions within the Nef sequence, such as regions that are necessary for modulation of protein expression and/or MHC sequestration. Generally, Nef mRNA expression is spatiotemporally regulated to be synchronous with basal levels of latent viral activation, building up like a reservoir to replenish low-levels of Nef protein to maintain MHC sequestration. Without wishing to be bound by theory, in embodiments, because the Nef protein has not been demonstrated to exhibit a long half-life, the small RNA functions to target the Nef mRNA pool to prevent MHC sequestration. In embodiments, the dsRNA product functions to spread to other HIV-infected cells and similarly increase and/or restore cell surface MHC levels.

In embodiments, the small RNA interacts with an RNA-induced silencing complex (RISC) in the latently HIV-infected cell. RISC is a multiprotein complex that incorporates one strand of a small RNA (e.g., siRNA, miRNA, etc.) (see e.g., FIG. 6). RISC uses the small RNA as a template for recognizing complementary mRNA. In embodiments, when the small RNA-loaded RISC finds a complementary strand, it activates RNase and cleaves the mRNA. In embodiments, the interaction between the small RNA and the RISC in the latently HIV-infected cell results in amplification of the small RNA. In embodiments, the amplification of the small RNA results in further silencing of pNef and shRNA amplification.

In embodiments, the small RNA is configured to be trafficked from the latently HIV-infected cell to at least a second HIV-infected cell. The trafficking, in embodiments, is through a Systemic RNA Interference-defective Transmembrane Family Member 1 (SIDT-1) or SIDT-2 cell surface protein complex in the latently HIV-infected cell. SIDT-1 and SIDT-2 are RNA-gated cell surface channels that function to bidirectionally traffic double-stranded RNA (dsRNA) into and out of the cell (Elhassan, et al. “Homo sapiens Systemic RNA-Interference-defective-1 Transmembrane Family Member 1 (SIDT1) Protein Mediates Contact-dependent Small RNA Transfer and MicroRNA-21-driven Chemoresistance.” J. Bid. Chem. Vol. 287, No. 8, 2012: pp. 5267-5277) and (Liu, et al. “Honeysuckle-derived microRNA2911 inhibits tumor growth by targeting TGF-β1.” Chinese Medicine. Vol. 16, No. 49, 2021:1-9). SIDT-1/2 are widely expressed among cell types in the body, including immune cells. In embodiments, the RISC-amplified shRNA and/or small RNA-Nef RNA duplex can be exported from the latently HIV-infected cell to at least a second cell HIV-infected cell via the SIDT-1/2 complex. For example and without wishing to be bound by theory, in embodiments, an RNAi template binds RISC in the latently HIV-infected cell, resulting in silencing of pNef and amplification of an shRNA; interfering shRNA oligos are then released via SIDT-1/2 for uptake by nearby HIV-infected cells.

In embodiments, administering the BioNV results in γδ T cell costimulation of NK cells in the subject. In embodiments, the BioNV results in restoration of the γδ T cell population in the subject. In embodiments, the γδ T cell is of a Vγ9JP repertoire.

In embodiments, administering the BioNV results in T cell-mediated cytotoxicity against one or more HIV-infected cells. In embodiments, administering the BioNV results in immunological memory developed against one or more HIV antigens.

Methods herein, in embodiments, reduce or minimize the opportunity for off-target effects in non-HIV infected cells, e.g., in comparison to a therapy that only targets cell surface markers, or a therapy that excises the entire HIV. For example, and without limitation, in embodiments, when a BioNV-delivered nucleic acid (e.g., a plasmid—containing the mechanistic properties to direct and pass it into the nucleus in a cell) is delivered to a non-infected cell, the gene editor will not be activated due to the gRNA specificity and the shRNA will not be activated due to the lack of the Nef mRNA sequence. However, Nef mRNA can be present in the cytosol of uninfected cells due to the uptake of Nef-containing exosomes shed from infected cells (both latent and active); in such a case, in embodiments, methods of treatment herein can target non-infected cells that uptake Nef. In embodiments, methods herein utilize a BioNV that reduces or minimizes the opportunity for off-target effects in non-HIV infected cells in comparison to a drug delivery platform that delivers checkpoint inhibitors, or other small molecules that can react with HIV and/or non-HIV targets.

In embodiments, methods of treatment herein include administering one or more HIV latency reversal agents (LRAs). LRAs, in embodiments, include PKC agonists (e.g., Prostratin Bryostatin-1 Ingenols: Ingenol-B, Ingenol 3, 20-dibenzoate (Ingenol-db), Ingenol-3-angelate (Ingenol mebutate, PEP005); MAPK agonists (e.g., Procyanidin trimer Cl); CCR5 antagonists (e.g., Maraviroc); Tat vaccine components (e.g., Tat Oyi vaccine, Tat-R5M4 protein); SMAC mimetics (e.g., SBI-0637142, Birinapant); Inducers of P-TEFb release (e.g., BETis: JQ1, I-BET, I-BET151, OTX015, UMB-136, MMQO, CPI-203, RVX-208, PFI-1, BI-2536, and B16727, HMBA); Activators of Akt Pathway (e.g., Disulfiram); Beonzotriazole derivatives (e.g., 1-hydroxybenzotraizol (HOBt)); Epigenetic modifiers (e.g., HDACis: TSA, trapoxin, SAHA, romidepsin, panobinostat, entinostat, givinostat, valproic acid, MRK-1/11, AR, fimepinostat, chidamide, HMTis: chaetocin, EPZ-6438, GSK-343, DZNEP, BIX-01294, UNC-0638); Immunomodulatory LRAs (e.g., TLR agonists: TLR2 (Pam3CSK4), TLR7 (GS-9620), TLR8, TLR9 (MGN 1703) agonists, IL-15 agonists (ALT-803), immune checkpoint inhibitors: ant-PD-1 (nivolumab, pembrolizumab), anti-CTLA-4 (ipilimumab). In embodiments, methods of treatment herein include administering LRAs (e.g., as a co-administration with a BioNV and/or encapsulated in a BioNV), for example as described in (AIT-AMMAR, et al., “Current Status of Latency Reversing Agents Facing the Heterogeneity of HIV-1 Cellular and Tissue Reservoirs,” Front Microbiol, Vol. 10, No. 3060, 2020: pp. 1-23).

In embodiments, the methods include administering one or more antiretroviral therapies. In embodiments, the BioNV encapsulates the one or more antiretroviral therapies, as described herein.

In embodiments, the antiretroviral therapy includes at least two, or at least three, or at least four of the antiretroviral therapies in combination.

In embodiments, methods of treatment, prevention, and/or amelioration of HIV include administration of a checkpoint inhibiting agent. In embodiments, the checkpoint inhibiting agent is an agent that modulates one or more of PD-1 (e.g., Cemiplimab, Nivolumab, Pembrolizumab), PD-L1 (e.g., Atezolizumab, Avelumab, Durvalumab), PD-L2, CTLA-4 (e.g., Ipilimumab), TIM-3 (e.g., MBG453, Sym023, TSR-022), LAG-3 (e.g., LAG525 (IMP701), REGN3767 (R3767), BI 754,091, tebotelimab (MGD013), eftilagimod alpha (IMP321), FS118), B7-H3/B7-H4 (e.g., MGC018, FPA150), A2aR (e.g., EOS100850, AB928), CD73 (e.g., CPI-006), NKG2A (e.g., Monalizumab), PVRIG/PVRL2 (e.g., COM701), CEACAMI (e.g., CM24), CEACAM 5/6 (e.g., NEO-201), FAK (e.g., Defactinib), CCL2/CCR2 (e.g., PF-04136309), LIF (e.g., MSC-1), CD47/SIRPα (e.g., Hu5F9-G4 (5F9), ALX148, TTI-662, RRx-001), CSF-1(M-CSF)/CSF-1R (e.g., Lacnotuzumab (MCS110), LY3022855, SNDX-6352, emactuzumab (RG7155), pexidartinib (PLX3397)), IL-1/IL-1R3 (e.g., CAN04, Canakinumab (ACZ885)), IL-8 (e.g., BMS-986253), SEMA4D (e.g., Pepinemab (VX15/2503)), Ang-2 (e.g., Trebananib), CLEVER-1 (e.g., FP-1305), Axl (e.g., Enapotamab vedotin (EnaV), and/or Phosphatidylserine (e.g., Bavituximab) (Marin-Acevedo, et al. “Next generation of immune checkpoint inhibitors and beyond.” J Hematol Oncol. Vol. 14, No. 45, 2021: pp. 1-29).

In embodiments, the methods described herein cause the excision of viral nucleic acids from one or more HIV-infected cells, optionally latently HIV-infected cells. In embodiments, the methods described herein cause a prevention of clonal expansion of HIV infected cells. In embodiments, methods described herein cause eradication, stoppage, halt or end of HIV and/or infection symptoms, or the progression of the symptoms or virus in the subject. In embodiments, methods described herein induce and/or maintain sustained viral control, optionally selected from undetectable levels of plasma viremia and/or maintenance of less than about 1000 copies/mL of HIV viral RNA or cDNA, less than about 500 copies/mL of HIV viral RNA or cDNA, less than about 100 copies/mL of HIV viral RNA or cDNA, less than about 50 copies/mL of HIV viral RNA or cDNA, or less than about 10 copies/mL of HIV viral RNA or cDNA, optionally as assayed by a polymerase chain reaction (PCR) test, a branched chain DNA (bDNA) test or a nucleic acid sequence-based amplification (NASBA) test.

### Dosing and Administration

The dosage of any BioNVs disclosed herein as well as the dosing schedule can depend on various parameters and factors, including, but not limited to, the specific marker-targeted BioNV, the severity of the condition, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the disease being treated, the severity of the disorder, and the anatomical location of the disorder. Some variations in the dosage can be expected.

In embodiments, delivery of BioNVs can be like that of a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989)).

Methods of treatment using BioNVs described herein, in embodiments, include dosage ranges in concentration of BioNVs per kilogram (kg) subject body weight. Suitable dosage ranges for methods of treatment described herein can include from about 103 BioNVs/kg to about 10¹² BioNVs/kg. In embodiments, the BioNVs are present in the composition at a concentration of about 103 BioNVs/mL to about 10¹⁴ BioNVs/mL. Alternatively, in embodiments, BioNV compositions are present in compositions as weight/volume in the range of about 5 ng/mL to about 500 mg/mL, or weight/subject body weight in the range of at least about 1 ng/kg to at least about 10 mg/kg.

Methods of treatment using BioNVs described herein, in embodiments, include intravenous, intramuscular, or parenteral administration, i.e., BioNVs can be infused into a subject via infusion or injection into the subject's blood or tissues.

In embodiments, BioNVs disclosed herein are administered by a controlled-release or a sustained-release means or by delivery of a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled or sustained-release of one or more active ingredients using, for example, hydropropylmethyl cellulose, other polymer matrices, gels, permeable membranes, osmotic systems, multilayer coatings, microparticles, microspheres, or a combination thereof, to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials are used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In embodiments, a controlled-release system is placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

In embodiments, the methods using BioNVs include applying BioNVs to a surface of a device (e.g., a catheter) or contained within a pump, patch, or other drug delivery device. The excipient or carrier can be selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In embodiments, BioNVs can be administered at doses that are congruent to dosages of whole cells, for example, based on CAR concentration. In embodiments, the typical concentration range of CAR protein per microgram of T cells is between 0.20 ng-0.70 ng, whereas a single BioNV may have a total number of CARs that is 5 times to 10,000 times less than the whole cell, resulting in a conversion of BioNV mass to CAR concentration, where the CAR concentration can be assumed equivalent (such as the case in exosomes) or increased (such as the case in BNVs) to the cell from which it originated (e.g., the T cell). In embodiments, the concentration and/or surface density of the targeting agent (e.g., CAR) is increased on the BioNV compared to the whole cell from which is it derived. In embodiments, the concentration and/or surface density of the targeting agent (e.g., CAR) is enriched by serial extrusion processing of the whole cell. In embodiments, the concentration and/or surface density of the targeting agent (e.g., CAR), among other cell surface molecules, on the BioNV is 2-fold to 100-fold increased relative to the whole cell. In embodiments, due to exosomes being naturally shed, their concentration and/or surface density of the targeting agent (e.g., CAR), among other cell surface molecules, is substantially the same as the whole cell.

The dosage regimen utilizing any BioNVs disclosed herein can be selected in accordance with a variety of factors including type, species, age, weight, sex, and medical condition of the subject; the severity of the condition to be treated; the route of administration; the renal or hepatic function of the subject; the pharmacogenomic makeup of the individual; and the specific composition of the invention employed. Any BioNVs disclosed herein can be administered in a single daily dose, or the total daily dosage can be administered in divided doses of two, three, or four times daily. Furthermore, any BioNVs disclosed herein can be administered continuously rather than intermittently throughout the dosage regimen.

In embodiments, BioNVs are administered in consecutive doses about every hour, about every 2 hours, about every 6 hours, about every 12 hours, about every 24 hours, about every 2 days, about every 4 days, about every 7 days, about every 2 weeks, about every 4 weeks, about every month, about every 2 months, about every 6 months, or about every year.

In embodiments, a combined remission or clinical remission of the infection, or non-detection of viral DNA or RNA is achieved within about 1 year, about 6 months, about 24 weeks, about 18 weeks, about 12 weeks, about 8 weeks, about 6 weeks, about 4 weeks, about 2 weeks, or about 1 week from administration of the composition and methods with such compositions.

### Compositions

In aspects, the present disclosure relates to compositions for the treatment of HIV infection comprising an allogeneic, hypoimmunogenic BioNV with a membrane-embedded targeting agent (e.g., CAR, VERR/viral ligand) targeted against a marker of an HIV-infected cell (e.g., Siglec-1, CD2, CD3, CD4, CCR5, CD20, CD25, CD30, CD32a, CD69, CD91, CD160, CD257, LAG-3, CD147, CD231, CEACAMI, PLXNB2, HLA-DR, PD-1, CTLA-4, TIGIT, LAG-3, TIM-3, BTK, Cyclophilin B, Sec62, Rab10, and/or SPCS), which encapsulates a gene editing payload for specifically targeted HIV proviral genomic sequences.

In embodiments, compositions include BioNVs. In embodiments, compositions include a BioNV and at least one antiretroviral therapy. In embodiments, compositions include a BioNV and at least one gene editing payload and/or at least one checkpoint inhibitor encapsulated within the aqueous core. In embodiments, the composition comprises a therapeutically effective amount of the BioNVs and/or a therapeutically effective amount of at least one checkpoint inhibitor. In embodiments, the BioNV and checkpoint inhibitor(s) can be combined in solution or can be in separate solutions to be co-administered.

In embodiments, the composition is allogeneic and/or hypoimmunogenic. In embodiments, the composition is derived from iPSCs (among other cell types) which have been modified to reduce expression of immunogenic molecules and/or increase expression of immunoprotective molecules.

In embodiments, the composition is hypoimmunogenic. For example, in embodiments, the composition does not result in an inflammatory reaction and/or an immune response upon administration. In embodiments, the BioNVs are hypoimmunogenic. In embodiments, upon administration to a subject, the composition, optionally the BioNVs therein, elicits less than about 50%, about 45%, about 40%, about 35%, about 30%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8, about 7%, about 6%, about 5%, about 4%, about 3%, about 2%, or about 1% of an inflammatory or immune response measured as a function of cytokine, chemokine, or immunomodulatory enzyme concentration, such as IL-1, IL-2, IL-3, IL-4, IL-6, IL-7, IL-9, IL-10, IL-11, IL-12, IL-13, IL-14, IL-15, IL-16, IL-17, IL-20, IFN-α/β/γ, TNFα/β, IDO, HLA-G, HGF, PGE2, among others, or any combination thereof, in comparison, e.g. to a cognate whole cell therapy counterpart.

In embodiments, the BioNVs are present in the composition at a concentration of about 103 NVs/mL to about 10¹⁴ NVs/mL. Alternatively, in embodiments, BioNV compositions are present in compositions as weight/volume in the range of about 5 ng/mL to about 500 mg/mL.

In embodiments, the composition is substantially free of one or more bacteria, virus, fungus, spore, mycoplasma, pyrogen, and in more particular embodiments, is substantially free of all the foregoing. In embodiments, the composition is substantially free of whole cells and intracellular cell components including organelles such as nuclei, mitochondria, Golgi, etc., and/or substantially free of non-targeting agent ligand-expressing NVs and/or substantially free of ruptured, damaged NVs. In embodiments, the composition is substantially free of cellular chromatin, nucleosomes, and other genetic material and non-therapeutic gene editing nucleic acids. In embodiments, BioNVs and BioNV compositions are substantial free of cellular genomic DNA.

### Pharmaceutical Compositions and Formulations

In aspects, the composition is a pharmaceutical composition. In embodiments, the pharmaceutical compositions of the present invention are formulated to provide a therapeutically effective amount of BioNVs and/or gene editing payloads as the active ingredient. In embodiments, the pharmaceutical compositions of the present invention are formulated to provide a therapeutically effective amount of one or more checkpoint inhibitors as a payload within a BioNV as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea, and the like. In addition, auxiliary, stabilizing, thickening, lubricating, and coloring agents can be used. Pharmaceutically acceptable excipients are generally sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Any composition disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In embodiments, the composition comprises an excipient or carrier. In embodiments, the diluent is a pharmaceutically acceptable excipient or carrier.

In embodiments, the pharmaceutical composition comprises a pharmaceutically acceptable diluent. Non-limiting example of diluents include liquid diluents such as water, ethanol, propylene glycol, glycerin, and various combinations thereof, and inert solid diluents such as calcium carbonate, calcium phosphate or kaolin. In embodiments, the diluent comprises one or more of saline, phosphate buffered saline, Dulbecco's Modified Eagle Medium (DMEM), alpha modified Minimal Essential Medium (alpha MEM), Roswell Park Memorial Institute Media 1640 (RPMI Media 1640), HBSS, human albumin, Ringer's solution, and the like, or any combination thereof.

In embodiments, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. In embodiments, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. In embodiments, the resulting compositions can include additional agents, such as preservatives, cryopreservatives (e.g., DMSO), and/or lyoprotectants (e.g., polyols, salts). In embodiments, the carrier can be, or can include a lipid-based or polymer-based colloid. In embodiments, the carrier material can be a colloid formulated as a liposome, a hydrogel, a microparticle, a nanoparticle, or a block copolymer micelle. In embodiments, the carrier material can form a capsule, and that material may be a polymer-based colloid.

In embodiments, the pharmaceutical compositions comprising the BioNVs include a solubilizing agent. In embodiments, the pharmaceutical compositions comprising the BioNVs include a cryoprotective agent or an agent to improve thermal stability, such as DMSO or glycerol. The pharmaceutical compositions, in embodiments, can be delivered with a suitable vehicle or delivery device as known in the art.

In embodiments, the composition comprises a scaffold. In embodiments, the scaffold comprises biomaterials. In a non-limiting example, the three-dimensional biomaterials include BioNVs embedded in an extracellular matrix attached to, or dispersed within, or trapped within the scaffold. In embodiments, the biomaterials are biodegradable and/or synthetic.

In embodiments, the scaffold comprises biodegradable biomaterials. Non-limiting examples of biodegradable biomaterials include fibrin, collagen, elastin, gelatin, vitronectin, fibronectin, laminin, reconstituted basement membrane matrix, starch, dextran, alginate, hyaluron, chitin, chitosan, agarose, sugars, hyaluronic acid, poly (lactic acid), poly (glycolic acid), polyethylene glycol, decellularized tissue, self-assembling peptides, polypeptides, glycosaminoglycans, derivatives and mixtures thereof. Other useful biodegradable polymers or polymer species include, but are not limited to, polydioxanone, polycarbonate, polyoxalate, poly (α-ester), polyanhydride, polyacetate, polycaprolactone, poly (ortho Esters), polyamino acids, polyamides, and mixtures and copolymers thereof, L-lactic acid and D-lactic acid stereopolymers, copolymers of bis (para-carboxyphenoxy) propanoic acid and sebacic acid, sebacic acid copolymers, caprolactone Copolymer, poly (lactic acid)/poly (glycolic acid)/polyethylene glycol copolymer, polyurethane and poly (lactic acid) copolymer, polyurethane and poly (lactic acid) copolymer, α-amino acid copolymer, α-amino acid and caproic acid copolymer, A-benzylglutamate and polyethylene glycol copolymers, succinate and poly (glycol) copolymers, polyphosphazenes, polyhydroxy-alkanoates and mixtures thereof. Binary and ternary systems are also contemplated. In embodiments, the scaffold comprises one or more of collagen, various proteoglycans, alginate-based substrates, and chitosan. In embodiments, the scaffold comprises one or more of a hydrogel, silk, Matrigel, acellular and/or decellarized scaffolds, poly-E-caprolactone scaffolds, resorbable scaffolds, and nanofiber-hydrogel composite.

In embodiments, the scaffold comprises synthetic biomaterials. Non-limiting examples of synthetic biomaterials include lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters.

In embodiments, the compositions can be prepared in any manner well known in the pharmaceutical arts, and can be administered by a variety of routes (e.g., subcutaneous, intravenous, etc.) depending upon whether local or systemic treatment is desired and upon the area to be treated. In embodiments, administration can be topical (including ophthalmic and to mucous membranes including intranasal, vaginal, and rectal delivery), pulmonary (e.g., by inhalation or insufflation of powders or aerosols, including by nebulizer; intratracheal, intranasal, epidermal and transdermal), ocular, oral, or parenteral. In embodiments, methods can include ocular delivery, topical administration (eye drops), subconjunctival, periocular or intravitreal injection or introduction by balloon catheter or ophthalmic inserts surgically placed in the conjunctival sac. In embodiments, parenteral administration includes intravenous, intra-arterial, subcutaneous, intraperitoneal or intramuscular injection or infusion; or intracranial, e.g., intrathecal or intraventricular administration. In embodiments, parenteral administration can be in the form of a single bolus dose, or may be, for example, by a continuous perfusion pump.

In embodiments, pharmaceutical compositions and formulations for topical administration may include transdermal patches, ointments, lotions, creams, gels, drops, suppositories, sprays, liquids, powders, and the like. In embodiments, methods of treating and/or preventing cancer include the use of pharmaceutical carriers, aqueous, powder or oily bases, thickeners, and the like.

In embodiments, the pharmaceutical compositions contain, as the active ingredient, nucleic acids and vectors described herein in combination with one or more pharmaceutically acceptable carriers. In embodiments, the terms “pharmaceutically acceptable” (or “pharmacologically acceptable”) refer to molecular entities and compositions that do not produce an adverse, allergic or other untoward reaction, when administered to an animal or a human, as appropriate. The methods and compositions disclosed herein can be applied to a wide range of species, e.g., humans, non-human primates (e.g., monkeys), horses or other livestock, dogs, cats, ferrets or other mammals kept as pets, rats, mice, or other laboratory animals. In embodiments, the term “pharmaceutically acceptable carrier,” includes any and all solvents, dispersion media, coatings, antibacterial, isotonic and absorption delaying agents, buffers, excipients, binders, lubricants, gels, surfactants and the like, that may be used as media for a pharmaceutically acceptable substance.

In embodiments, the compositions can be applied to a surface of a device (e.g., a catheter) or contained within a pump, patch, or other drug delivery device. In embodiments, the compositions can be administered alone, or in a mixture, in the presence of a pharmaceutically acceptable excipient or carrier (e.g., physiological saline). The excipient or carrier is selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In embodiments, the pharmaceutical composition further comprises a therapeutically effective amount of one or more agents for targeting PD-1. In embodiments, the one or more agents for targeting PD-1 is an antibody or antibody format. In embodiments, the one or more agents for targeting PD-1 is selected from nivolumab, pembrolizumab, and pidilizumab.

In embodiments, the pharmaceutical composition further comprises a therapeutically effective amount of one or more agents for targeting PD-L1. In embodiments, the one or more agents for targeting PD-L1 is an antibody or antibody format. In embodiments, the one or more agents for targeting PD-L1 is selected from atezolizumab, avelumab, durvalumab, and BMS-936559.

In embodiments, the pharmaceutical composition further comprises a therapeutically effective amount of one or more agents for targeting CTLA-4. In embodiments, the one or more agents for targeting CTLA-4 is an antibody or antibody format. In embodiments, the one or more agents for targeting CTLA-4 is selected from ipilimumab, tremelimumab, AGEN1884, and RG2077.

In embodiments, the compositions, e.g., pharmaceutical compositions, disclosed herein are suspended in a saline buffer (including, without limitation, TBS, PBS, and the like).

The present technology includes the disclosed BioNVs in various formulations of pharmaceutical compositions. BioNVs disclosed herein, in embodiments, can take the form of solutions, suspensions, emulsion, drops, tablets, pills, pellets, capsules, capsules containing liquids, powders, sustained-release formulations, emulsions, aerosols, sprays, suspensions, or any other form suitable for use.

Pharmaceutical compositions comprising the BioNVs described herein may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any BioNVs disclosed herein are formulated in accordance with routine procedures as a pharmaceutical composition adapted for a mode of administration disclosed herein.

### Additional Therapeutic Agents

In embodiments, the compositions or methods described herein further comprise a therapeutically effective amount of one or more additional therapeutic agents. In embodiments, the therapeutically effective amount of one or more additional therapeutic agents may be in solution with a BioNV, adsorbed onto the surface of the NV, or a payload encapsulated within a BioNV. In embodiments, the additional therapeutic agent is one or more of a checkpoint inhibitor, an analgesic, an antiretroviral therapeutic agent, a latency reversal agent, and/or an anti-infective agent.

In embodiments, the additional therapeutic agent in the composition comprises a therapeutically effective amount of one or more antiretroviral therapeutic agents. In embodiments, the antiretroviral therapy includes a non-nucleoside reverse transcriptase inhibitor (NNRTI), optionally selected from efavirenz (SUSTIVA), rilpivirine (EDURANT) and doravirine (PIFELTRO). In embodiments, the antiretroviral therapy includes a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY). In embodiments, the antiretroviral therapy includes a protease inhibitor (PI), optionally selected from atazanavir (REYATAZ), darunavir (PREZISTA), and lopinavir/ritonavir (KALETRA). In embodiments, the antiretroviral therapy includes an integrase inhibitor, optionally selected from bictegravir sodium/emtricitabine/tenofovir alafenamide fumar (BIKTARVY), raltegravir (ISENTRESS), and dolutegravir (TIVICAY). In embodiments, the antiretroviral therapy includes an entry or fusion inhibitor, optionally selected from enfuvirtide (FUZEON) and maraviroc (SELZENTRY). In embodiments, the antiretroviral therapy comprises at least two, or at least three, or at least four of the antiretroviral therapies in combination. In embodiments, the BioNV encapsulate one or more different antiretroviral therapeutic agents. In embodiments, the BioNV composition includes one or more different ART agents outside of the NV.

In embodiments, the additional therapeutic agent in the composition comprises a therapeutically effective amount of one or more HIV latency reversal agents (LRAs). LRAs, in embodiments, include PKC agonists (e.g., Prostratin Bryostatin-1 Ingends: Ingenol-B, Ingenol 3, 20-dibenzoate (Ingenol-db), Ingenol-3-angelate (Ingenol mebutate, PEP005); MAPK agonists (e.g., Procyanidin trimer C1); CCR5 antagonists (e.g., Maraviroc); Tat vaccine components (e.g., Tat Oyi vaccine, Tat-R5M4 protein); SMAC mimetics (e.g., SBI-0637142, Birinapant); Inducers of P-TEFb release (e.g., BETis: JQ1, I-BET, I-BET151, OTX015, UMB-136, MMQO, CPI-203, RVX-208, PFI-1, BI-2536, and BI6727, HMBA); Activators of Akt Pathway (e.g., Disulfiram); Beonzotriazole derivatives (e.g., 1-hydroxybenzotraizol (HOBt)); Epigenetic modifiers (e.g., HDACis: TSA, trapoxin, SAHA, romidepsin, panobinostat, entinostat, givinostat, valproic acid, MRK-1/11, AR, fimepinostat, chidamide, HMTis: chaetocin, EPZ-6438, GSK-343, DZNEP, BIX-01294, UNC-0638); Immunomodulatory LRAs (e.g., TLR agonists: TLR2 (Pam3CSK4), TLR7 (GS-9620), TLR8, TLR9 (MGN 1703) agonists, IL-15 agonists (ALT-803), immune checkpoint inhibitors: anti-PD-1 (nivolumab, pembrolizumab), anti-CTLA-4 (ipilimumab). In embodiments, methods of treatment herein include administering LRAs (e.g., as a co-administration with a BioNV and/or encapsulated in a BioNV), for example as described in (AIT-AMMAR, et al., 2020).

In embodiments, the present compositions or methods contemplate other additional therapeutic agents, for example, an analgesic, to aid in treating inflammation or pain at the site of the administration, or an anti-infective agent to prevent infection of the site of treatment with the composition. Non-limiting examples of additional therapeutic agents include analgesics, such as nonsteroidal anti-inflammatory drugs, opiate agonists and salicylates; anti-infective agents, such as anthelmintics, antianaerobics, antibiotics, aminoglycoside antibiotics, antifungal antibiotics, cephalosporin antibiotics, macrolide antibiotics, miscellaneous B-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens, immunobiological agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents, minerals; vitamins, such as vitamin A, vitamin B, vitamin C, vitamin D, vitamin E, and vitamin K; and radionuclides such as Yttrium-90, Iodine-131, Samarium-153, Lutetium-177, Astatine-211, Lead-212/bismuth-212, Radium-223, Actinium-225, and Thorium-227.

Additional non-limiting examples of useful therapeutic agents from the above categories include: (1) analgesics in general, such as lidocaine or derivatives thereof, and nonsteroidal anti-inflammatory drugs (NSAIDs) analgesics, including diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) Hi-blocker antihistamines, such as clemastine and terfenadine; (5) anti-infective agents, such as mupirocin; (6) antianaerobic anti-infectives, such as chloramphenicol and clindamycin; (7) antifungal antibiotic anti-infectives, such as amphotericin b, clotrimazole, fluconazole, and ketoconazole; (8) macrolide antibiotic anti-infectives, such as azithromycin and erythromycin; (9) miscellaneous B-lactam antibiotic anti-infectives, such as aztreonam and imipenem; (10) penicillin antibiotic anti-infectives, such as nafcillin, oxacillin, penicillin G, and penicillin V; (11) quinolone antibiotic anti-infectives, such as ciprofloxacin and norfloxacin; (12) tetracycline antibiotic anti-infectives, such as doxycycline, minocycline, and tetracycline; (13) antituberculosis antimycobacterial anti-infectives such as isoniazid (INH), and rifampin; (14) antiprotozoal anti-infectives, such as atovaquone and dapsone; (15) antimalarial antiprotozoal anti-infectives, such as chloroquine and pyrimethamine; (16) anti-retroviral anti-infectives, such as ritonavir and zidovudine; (17) antiviral anti-infective agents, such as acyclovir, ganciclovir, interferon alfa, remdesivir, and rimantadine; (18) antifungal topical anti-infectives, such as amphotericin B, clotrimazole, miconazole, and nystatin; (19) antiviral topical anti-infectives, such as acyclovir; (20) electrolytic and renal agents, such as lactulose; (21) loop diuretics, such as furosemide; (22) potassium-sparing diuretics, such as triamterene; (23) thiazide diuretics, such as hydrochlorothiazide (HCTZ); (24) uricosuric agents, such as probenecid; (25) enzymes such as RNase and DNase; (26) antiemetics, such as prochlorperazine; (27) salicylate gastrointestinal anti-inflammatory agents, such as sulfasalazine; (28) gastric acid-pump inhibitor anti-ulcer agents, such as omeprazole; (29) H2-blocker anti-ulcer agents, such as cimetidine, famotidine, nizatidine, and ranitidine; (30) digestants, such as pancrelipase; (31) prokinetic agents, such as erythromycin; (32) ester local anesthetics, such as benzocaine and procaine; (33) musculoskeletal corticosteroid anti-inflammatory agents, such as beclomethasone, betamethasone, cortisone, dexamethasone, hydrocortisone, and prednisone; (34) musculoskeletal anti-inflammatory immunosuppressives, such as azathioprine, cyclophosphamide, and methotrexate; (35) musculoskeletal nonsteroidal anti-inflammatory drugs (NSAIDs), such as diclofenac, ibuprofen, ketoprofen, ketorlac, and naproxen; (36) minerals, such as iron, calcium, and magnesium; (37) vitamin B compounds, such as cyanocobalamin (vitamin B12) and niacin (vitamin B3); (38) vitamin C compounds, such as ascorbic acid; and (39) vitamin D compounds, such as calcitriol.

### BioNVs

In aspects, the present invention includes BioNVs. In embodiments, BioNVs are approximately 10-1200 nm in size which contain outwardly facing, membrane-embedded targeting agents capable of binding a target molecule. Without wishing to be bound by theory, in embodiments, the biomimetic quality owes to the nanovesicle composition which originates from the plasma membrane of allogeneic, hypoimmunogenic modified cells. In embodiments, BioNVs comprise plasma membrane-derived lipid bilayers, fully encapsulating an aqueous core which can house a variety of gene editing payloads and small RNAs.

To ensure proper directionality of targeting agents and to eliminate BioNVs lacking targeting agents, in embodiments, HPLC-based affinity chromatography techniques can be used to select and concentrate only the BioNVs with a sufficient surface concentration of solvent-exposed targeting agents. HPLC-based affinity chromatography techniques can be used to reduce the concentration of contaminating cell material and NVs which harbor immunogenic cell surface markers, either by positive or negative selection.

In embodiments, the BioNV targeting agent constructs can include a variety of structural molecules, such as fusion proteins that are typically used in a chimeric antigen receptor (CAR). The structure-function of a prototypical CAR includes a fusion protein comprising an extracellular (or outwardly facing) binding moiety (e.g., scFv), connected by a hinge peptide (e.g., CH2/CH3 domains from an IgG Fc region, Gly-Gly-Ser peptide linkage, CD28 peptide, CD8a peptide, etc.) to a transmembrane domain (e.g., CD28, CD3ζ, CD4, CD8α, ICOS, etc.), followed by a variety of intracellular signaling domains (e.g. 4-1BB, CD3ζ, CD28, 4-1BB, ICOS, CD27, OX40, etc.). In embodiments, BioNVs lack the intracellular machinery of whole cells and therefore the CAR does not necessitate any intracellular signaling molecules. In embodiments, the targeting agent construct includes an extracellular binding moiety fused with an IgG CH2/CH3 linker to a CD28 transmembrane domain and substantially lacks any intracellular domains or functionality. In embodiments, the targeting agent construct can be a fusion protein with Vp1 AAV and can have the prototypical intracellular domains swapped or otherwise fused to anchor proteins, e.g., PLA2 domain from an AAV, fusion proteins, cytoskeletal elements, small molecule transporting domains, etc., which may aid in the fusion to target cells and/or packaging and release of therapeutic payloads.

In embodiments, the BioNV targeting agent antigen-binding molecules include a variety of targeting agents, including antibody-based or antibody format binding domains. In embodiments, BioNVs comprise antibody or antibody format binding moieties selected from one or more of a monoclonal antibody, polyclonal antibody, antibody fragment, VERR, viral ligand, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody. In embodiments, the targeting agent construct includes binding moieties with a Bispecific T cell Engager (BiTE), variable heavy chain IgG fragment V_{H}H, V_{NAR}, or through an engineered T Cell Receptor (TCR). In embodiments, the targeting agent is a fusion with a scFv of a heavy chain (HC) or light chain (LC) variable portion.

In embodiments, BioNVs are formed by disrupting the cell membranes of engineered iPSCs. In embodiments, the hypoimmunogenic iPSCs are characterized by a B2M−/−, CIITA−/−, CD47+/+, PD1−/− plasma membrane profile and may be used to generate the present BioNVs. In embodiments, BioNVs can be generated from the parent iPSC cell line via sonication, adaptive focused acoustics technology, French press, extrusion, serial extrusion, cell lysis by detergent, and electroporation, among other methods. In embodiments, serial extrusion is the method used to generate BioNVs.

In embodiments, BioNVs can selected for homogeneity of size by dynamic light scattering (DLS), flow cytometry, mass photometry, among other methods of determining particle size. In embodiments, BioNVs can be filtered for a particle size, or range of sizes, to optimize renal clearance and other clinically-relevant NV properties. In embodiments, BioNVs can range in size between about 20 nm to 1200 nm in size. In embodiments, BioNVs are about 10 nm in size, about 20 nm in size, about 30 nm in size, about 40 nm in size, about 50 nm in size, about 60 nm in size, about 70 nm in size, about 80 nm in size, about 90 nm in size, about 100 nm in size, about 120 nm in size, about 140 nm in size, about 160 nm in size, about 180 nm in size, about 200 nm in size, about 300 nm in size, about 400 nm in size, about 500 nm in size, about 600 nm in size, about 700 nm in size, about 800 nm in size, about 900 nm in size, about 1000 nm in size, about 1100 nm in size, or about 1200 nm in size. BioNVs range in size from about 10 nm to 20 nm in size, about 20 nm to 30 nm in size, about 30 nm to 40 nm in size, about 40 nm to 50 nm in size, about 50 nm to 60 nm in size, about 60 nm to 70 nm in size, about 70 nm to 80 nm in size, about 80 nm to 90 nm in size, about 90 nm to 100 nm in size, about 10 nm to 100 nm in size, about 100 nm to 200 nm in size, about 200 nm to 400 nm in size, about 400 nm to 600 nm in size, about 600 nm to 800 nm in size, about 800 nm to 1000 nm in size, or about 1000 nm to 1200 nm in size.

iPSC-derived BioNVs, in embodiments, include NVs with an outer plasma membrane leaflet only, an inner plasma membrane leaflet only, and/or both leaflets of a plasma membrane lipid bilayer intact. iPSC-derived NVs, in embodiments, include additional lipid additives (e.g., phosphatidylethanolamine, phosphatidylcholine, phosphatidylinositols, ceramides, lecithin, etc.), non-ionic surfactants (e.g., sorbitan monostearate, octadecylamine, etc.), sterols (e.g., cholesterol, bile salt derivatives, etc.), polyols (e.g., maltodextrin, sorbitol, sucrose, mannitol, etc.) and proteins (e.g., serum albumin, etc.) added for improved physicochemical properties, such as thermal stability and therapeutic payload packaging/release. The amount of cholesterol and the length and saturation of the hydrocarbon chains of the phospholipids can affect the rigidity and the stability of the bilayer, and in turn the capability of the NVs to host and release drugs, biomolecules, and other therapeutic payloads. In embodiments, BioNVs also incorporate zwitterionic lipids and methods of using zwitterionic lipids, for example, as described in US Patent Publication No. US 20130216607, the contents of which are herein incorporated by reference in its entirety. Correspondingly, functionalization of the hydrophilic heads of the lipids with polymers or biomolecules can provide additional features to the vesicle surface, thus shaping their interaction with blood components, tissues, and the immune system in vivo.

In embodiments, targeting agent targets include a variety of cell surface markers, including markers of cellular infection with HIV. In embodiments, in the context of targeting latently HIV-infected immune cells, sialic acid-binding immunoglobulin-like lectins (Siglecs), such as Siglec-1, is the effective target. Siglecs can be divided into subsets based on sequence and structure similarity, such as CD33-related Siglecs (e.g., Siglec-H, Siglec-5, and Siglec-14) and CD22-related Siglecs.

In embodiments, the targeting agent includes a receptor and fusion peptide combined in a single complex, such as a VERR/viral receptor/ligand complex. In embodiments, the VERR/viral ligand does not contain combined fusion and recognition functionality. In embodiments, the VERR/viral ligand has the same cell and/or tissue tropism as the virus from which the VERR/viral ligand originates. In embodiments, the VERR/viral ligand has altered cell and/or tissue tropism compared to the virus from which the VERR/viral ligand originates. In embodiments, the VERR/viral ligand facilitates endosomal uptake. In embodiments, the VERR/viral ligand facilitates membrane fusion (fusogen).

In embodiments, the VERR/viral ligand comprises a protein that targets cell adhesion molecules (CAMs). A majority of viral receptors identified to date are CAMs that function in cell-cell and cell-to-extracellular matrix adhesion and are essential mediators of cellular processes such as development, maintenance of cellular structure, cell signaling, and maintenance and repair of tissues. The family of CAMs includes selectins, cadherins, integrins, and IgSF members. The ubiquitous expression and multifactorial function of CAMs in ligand binding, endocytosis, and signaling provides a multitude of cell targeting mechanisms for BioNVs to engage CAMs. In embodiments, viral ligands include fusions, variants, or portions of the viral ligands of HIV, measles virus, reovirus, rhinovirus, adenovirus, poliovirus, and coxsackievirus B (CVB) (IgSF receptor) that are known to engage CAM receptors.

In embodiments, the BioNVs comprise one of more VERR and/or viral ligand and/or host cell receptor components of Table 1.

TABLE 1
Exemplary viral ligand-cell receptor for
VERR/viral ligand targeted BioNVs.

| Viral Ligand | Host Cell Receptor |
|---|---|
| Influenza glycoproteins hemagglutinin (HA) | Sialic acid (SA) receptors |
| and neuraminidase (NA); H1, H7, H10 | |
| HIV ENV (gp120/gp41) | CD4, CXCR/CCR5 |

IgSF members have emerged as receptors for a wide range of viruses including enveloped and nonevenveloped viruses, including reovirus, adenovirus, coxsackievirus, rabies virus, measles virus, and HIV. In embodiments, the VERR/viral ligand comprises a viral protein that targets the IgSF member CD4 as the primary receptor, e.g., as is the case with HIV, which also requires specific coreceptors CCR4 and CXCR5. In embodiments, the VERR/viral ligand can include the HIV envelope (ENV) glycoprotein which interacts with CD4 to mediate specific interactions between ENV and CD4 that would facilitate entry into immune cells. ENV is a trimeric protein composed of gp120 and gp41, and gp120 mediates binding to CD4 through conserved domains leading to conformational changes within gp120 and CD4. Following CD4 interactions, the ENV viral ligand binding to the chemokine coreceptors CXCR4 or CCR5 can be used to specifically target BioNVs to macrophages and CD4+ T cells, respectively. However, initial interactions between ENV and host cells occur via nonspecific cellular receptors including heparan sulfate proteoglycans or specific receptors such as α4β7 integrin or the innate immune receptor DC-SIGN. In embodiments, the modified cell from which the BioNV is derived expresses a gp120/41 complex that incorporates mutations to prevent these nonspecific interactions. In embodiments, and without wishing to be bound by theory, the mutations produce a gp120/41 complex with reduced neural toxicity (Jadhav and Nema. “HIV-Associated Neurotoxicity: The Interplay of Host and Viral Proteins.” Mediators of Inflammation. Vol. 2021, No. 1267041, 2021:11 pages).

In embodiments, BioNVs are targeted to T cells by use of sialyllactose from gangliosides which can serve as the viral attachment factor for Sialyl glycan receptor expression on T cell subsets for targeting to immune cells, for example, via hemagglutinin protein selected from H1, H7 and H10 from Influenza A, or H1N1, H3N2, H7N9, and H10N8, among others. In embodiments, the BioNV is targeted to a cell via a viral fusogen or viral glycoprotein which facilitates fusion of the NV to the cell plasma membrane.

Those skilled in the art will understand that VERRs/viral ligands can be designed to target cell surface markers of any cell subset of interest and the appropriate methods for optimization.

In embodiments, the BioNVs are CD34+, or derived from CD34+ cells, such as human CD34+ cord blood. CD34+ cord blood-derived cell lines may serve as a base cell line for BioNV development, production, and manufacturing for the delivery of gene editing therapeutics.

In embodiments, the BioNVs and/or compositions comprising BioNVs are administered in combination with one or more additional compounds. In embodiments, the BioNVs are pretreated with one or more additional compounds, for example prior to administration to a subject.

In embodiments, BioNVs are modular and allogeneic (off-the-shelf) due to the lack of immunogenicity from engineered iPSCs. In embodiments, the lack of whole cell signaling components allows BioNVs to be easily tunable for target specificity and resistance to immunosuppression. In embodiments, BioNVs lack the genetic elements that contribute to runaway cytokine storms, minimizing patient risk of cytokine release syndrome (CRS). In embodiments, BioNVs are derived from cells capable of crossing biological barriers and/or have viral receptors for facilitating crossing.

Without wishing to be bound by theory, BioNVs generated from iPSC engineered allogeneic base cell lines represent immune invisible BioNVs which have the potential for multi-dosing. BioNV antibody-mediated neutralization is minimized, and immune cell-mediated clearance is evaded (T cell and macrophage). In embodiments, BioNVs do not contain viable genetic material from the cells they were derived that can cause CRS or teratoma. In embodiments, BioNVs are derived from different cell types with or without barrier penetrating ligands to further control activity post-infusion.

### Methods of BioNV Formation

In embodiments, the modified cell is a hypoimmunogenic cell derived from iPSCs that have been engineered to have reduced or ablated expression and/or activity of immunogenic proteins and/or express or have increase expression of immunoprotective proteins. In embodiments, iPSCs are reverted from a somatic state using microRNA technology in lieu of small molecule trans-activators. The use of microRNA provides a tighter differentiation system and that results in higher quality iPSCs. Without wishing to be bound by theory, these high quality iPSCs are less prone to expression dampening (of post-engineered proteins, such as CD47) and genetic drift, and possess higher culture splitting qualities/quantities (the cultures can be divided more times than other methods before cellular integrity issues occur).

In embodiments, BioNVs derived from iPSC-derived hypoimmunogenic cells which retain the functionality from the hypoimmunogenic cell, for example and without limitation, the ability to cross the blood-brain barrier, such as is the case of macrophages/monocytes, or tissue-specific factors such as is the case in cardiomyocytes, hepatocytes, etc.

In embodiments, allogeneic iPSCs have their MHC class I and MHC class II complexes disrupted by knocking out critical proteins involved in their expression, for example, B2M which is a serum protein found in association with the MHC class I heavy chain on the surface of nearly all nucleated cells that is involved in the presentation of peptide antigens to the immune system.

In embodiments, once the B2M knock-out (KO), CIITA KO, IL-6 KO, and CD47tg knock-in (KI) is engineered into the iPSC, the TRAC and TRBC genes can be knocked out. In embodiments, only one gene for each is knocked out rather than both of on the separate alleles. In embodiments, the TRAC and TRBC genes can be knocked out as described herein. The purpose of knocking out the TRAC and TRBC genes is to eliminate the T-cell receptors. In embodiments, the modified cell is differentiated to a T-cell subset which lacks T-cell receptors to derive the BioNVs. Genetically modifying the cells to substantially lack TCRs reduces the chances for a competing ligand to the targeting agent construct that can target non-specifically to alternate tissues. Therefore, in embodiments, the TCR genes are knocked-out as a strategy to reduce off-target effects of the BioNVs. In embodiments, TRAC/TRBC knock-outs decrease the likelihood of CRS, as well as BioNV toxicity, generally.

In embodiments, the modified cell is expanded after engineering; any small scale expansion or large-scale feeder system expansion methods known in the art can be used.

In embodiments, after the B2M KO, CIITA KO, IL-6 KO, CD47tg KI, and an IL-2 promoter-driven green fluorescence protein (GFP) (IL-2p GFP) reporter is constructed, the targeting agent constructs can be integrated/engineered into the cell. In embodiments, the targeting agent constructs can be knocked-in to the TRAC/TRBC genes, simultaneously knocking-out the remaining TRAC/TRBC genes, resulting in a cell that is targeting agent+ and TRAC/TRBC−/−. In embodiments, the targeting agent construct can be knocked-in to the TRAC/TRBC gene location on both loci simultaneously, resulting in a cell that is targeting agent+/+ and TRAC/TRBC−/−.

In embodiments, once the B2M KO, CIITA KO, IL-6 KO, CD47tg KI, IL-2p GFP KI, and targeting agent modified cells (e.g., iPSCs) are engineered, the Immunological Synapse (IS) quality is measured between the targeting agent recognition domains and the biomarker. In embodiments, the quality of the IS of BioNVs can be directly related to efficacy in whole cell therapies.

In embodiments, the BioNVs, or the hypoimmunogenic cell derived therefrom, comprises a nucleic acid encoding GFP (among other fluorescence proteins). In embodiments, once the B2M KO, CIITA KO, CD47tg KI, IL-6 KO, TRAC/TRBC single KOs are engineered into the iPSC, a GFP molecule is engineered into the modified cell line. In embodiments, this serves as the control cell line. In embodiments, the non-control cell line (the therapeutic cell line) does not have GFP. In embodiments, the nucleic acid encoding GFP is operably linked to a promoter from one or more of IL-2, perforin, granzyme, alarmin, TNF, INF, a combination thereof, and/or any other cell-specific gene or reporter gene. The IL-2 promoter is constitutively activated when lymphocytes are broadly/globally activated from various stimuli. In embodiments, a more focused activation/repression (regulation) is used. In embodiments, the IL-2p GFP reporter gene serves as an indicator for the degree of broad/global activation of the cell (as part of the BioNV derivation process). In embodiments, the GFP signal, coupled with immunoblot analysis of cytokine levels (such as perforins, granzymes, alarmins, TNFs, and INFs) allows efficient regulation of the degree of broad/global activation of a lymphocyte when exposed to activating antigens. In embodiments, GFP is used to compare the degrees of activation between manufacturing lots and ensure consistency for therapeutic development.

In embodiments, the hypoimmunogenic cells are CD34+, or derived from CD34+ cells, such as human CD34+ cord blood. In embodiments, CD34+ cord blood-derived cell lines may serve as a base cell line for BioNV development, production, and manufacturing for the delivery of gene editing therapeutics. In embodiments, a CD34+ cord blood-derived hypoimmunogenic cell line has been experimentally confirmed for the low expression of HLA 1/2 and overexpression of CD47 (Deuse, et al. “Hypoimmunogenic derivatives of induced pluripotent stem cells evade immune rejection in fully immunocompetent allogeneic recipients.” Nat Biotechnol. Vol. 37, 2019: 252-258).

In embodiments, the hypoimmunogenic cell can be engineered using multiple hypoimmunogenic engineering techniques, for example, as described in Deuse et al., Han et al., Xu et al., and Harding et al., and also as described in published U.S. patent applications US20190376045, 20190376045, 20210308183, and 20210292715 to Deuse, US20210161971 to Nagy, US20180141992 to Strominger, and Published European patent application 3693384 to Poirot, each of which is incorporated by reference herein in their entirety, (Han, et al. “Generation of hypoimmunogenic human pluripotent stem cells.” PNAS. Vol. 116, No. 21 2019: pp. 10441-10446.), (Xu, et al. “Targeted Disruption of HLA Genes via CRISPR-Cas9 Generates iPSCs with Enhanced Immune Compatibility.” Cell Stem Cell. Vol. 24, No. 4, 2019: pp. 566-578.), and (Harding, et al., “Induction of long-term allogeneic cell acceptance and formation of immune privileged tissue in immunocompetent hosts.” BioRxiv. 716571 [Preprint], Jul. 30, 2019.).

In embodiments, BioNVs are derived from cells which have eliminated HLA genes that encode the MHC membrane glycoproteins that confer immune reactions associated with GVHD rejections. The HLA gene clusters can be divided into three categories: 1) the MHC Class I pathway, 2) the MHC Class II pathway, and 3) the MHC Class Ill pathway. Only the MHC Class I and II pathways express the protein complexes elicit an immune response in GVHD, whereas MHC Class Ill complexes are not involved in immunization activities.

The elimination of the MHC classes of protein complexes can bigger NK cells and macrophages into an active clearance mode where the cells are subsequently destroyed. To avoid this kill mechanism, in embodiments, the addition of a CD47 isoform 2 transmembrane molecular protein tag can be engineered into the cell membrane of the modified cell to avoid natural killer and macrophage-mediated kill responses, for example, as described in Willingham et al., Deuse et al., and Han et al. (Willingham S B, et al. “The CD47-signal regulatory protein alpha (SIRPa) interaction is a therapeutic target for human solid tumors.” PNAS. Vol. 109, No. 17, 2012: pp. 6662-7.). In embodiments, cells can be engineered to use additional mechanisms to prevent these responses such as those described in: 1) the CD24 transmembrane molecular protein tags, for example as performed in Zhao et al. (Zhao W, et al. “Strategies for Genetically Engineering Hypoimmunogenic Universal Pluripotent Stem Cells.” iScience. Vol. 23, No. 6, 2020:101162.), 2) the membrane-bound surfactant protein-D (SP-D), for example as performed in Jiaravuthisan et al. (Jiaravuthisan P, et al. “A membrane-type surfactant protein D (SP-D) suppresses macrophage-mediated cytotoxicity in swine endothelial cells.” Transpl Immunol. Vol. 47, 2018: pp. 44-48.), and 3) the molecular PD-L1 tag for prevention of T-cell responses. In embodiments, PD-L1 is overexpressed in BioNVs derived from a cell that has not been activated and is not loaded with apoptotic cytokines. In embodiments hypoimmunogenic cells that are not to be activated have PD-L1 upregulated, i.e. for BioNVs used for gene editor delivery. In embodiments, CD47 can be utilized in genetically engineered iPSCs for immune tolerance to innate immune cells, for example, such as in Chhabra et al., Han et al., and Jaiswal et al. (Chhabra A, et al. “Hematopoietic stem cell transplantation in immunocompetent hosts without radiation or chemotherapy.” Sci Transl Med. Vol. 8, No. 351, 2016: 351ra105.) and (Jaiswal S, et al. “CD47 is upregulated on circulating hematopoietic stem cells and leukemia cells to avoid phagocytosis.” Cell. Vol. 138, No. 2, 2009: pp. 271-85.). In embodiments, cells can be modified as described in U.S. Pat. No. 8,562,997 to Jaiswal, et al., which is incorporated by reference herein in its entirety.

In embodiments, approaches can be used which do not entirely knock-out all HLA genes, for example, as performed in Xu et al. and Han et al., which only knock-out the HLA genes that are highly associated with an immune response, leaving intact the HLA genes that dampen a macrophage or NK response (e.g., HLA-E, HLA-F, and HLA-G). In embodiments, this approach does not require the addition of a CD47 tag; the modified cell can be engineered to generate BioNVs with or without CD47.

In embodiments, methods improve upon the approaches of hypoimmunogenicity of Table 2.

TABLE 2
Three methods of modification of cells using the HLA knockout combined with a
CD47 isoform 2 tag and a PD-L1 transmembrane tag (Zhao, et al.) (Gornalusse G
G, et al. “HLA-E-expressing pluripotent stem cells escape allogeneic responses
and lysis by NK cells.” Nat Biotechnol. Vol. 35, No. 8, 2017: pp. 765-772.).

| Source | HLA-A | HLA-B | HLA-C | HLA-E | HLA-F | HLA-G | HLA-II | CD47 | PD-L1 |
|---|---|---|---|---|---|---|---|---|---|
| Gornalusse, | Knockout | | | + | Knockout | | | | |
| 2017 | | | | | | | | | |
| Deuse, | B2M Knockout | | | | | | CIITA KO | + | |
| 2019 | | | | | | | | | |
| Xu, | Knockout | | | | | | CIITA KO | | |
| 2019 | | | | | | | | | |
| Han, | Knockout | | | | | | CIITA KO | + | + |
| 2019 | | | | | | | | | |

In embodiments, developing the allogeneic modified cell involves the removal of MHC Class I and MHC Cass II protein complexes through the disruption of certain HLA genes, or a B2M knockout, followed by knocking out the CIITA gene. In embodiments, the knockouts can be performed using CRISPR gene editing approaches, due to their rapid mechanism of action. In embodiments, the knockouts are performed using Zinc Finger Nucleases (ZFNs) and/or TALENS. In embodiments, Cre/Lox recombinase systems are used to generate the modified cell. In embodiments, RNA silencing (RNAi, shRNA, microRNA, CRISPR Cas13a-d, etc.) is used to generate the modified cell.

In embodiments, developing the allogeneic modified cell includes the Harding et al. methods of creating allogenicity that is distinct from the above methods. In embodiments, in lieu of deleting the MHC class I/II genes and running the risk of preventing long-term acceptance by the recipient, the Harding et al. method includes an alternate approach based on immune escape mechanisms that occurs in nature. In embodiments, the method relies on the Harding et al. biomimicry based on the horizontally transmitted cancer devil facial tumor disease (DFTD) type 2 that is predominant in Tasmanian devils. In embodiments, developing the allogeneic modified cell includes over-expression of the immunomodulatory proteins CCL21, PD-L1, FasL, SerpinB9, H2-M3, CD47, CD200, and/or MFG-E8 to protect cell derivatives from long-term immune rejection in mice (and humans), without the deletion of MHC class I/II proteins. In embodiments, the modified cell expresses one or more of the proteins shown in Table 3, including any splice variant and/or isoform of any of the indicated proteins (e.g., CD200 splice variants). In embodiments, this system can be used to interfere with the activity of antigen presenting cells (APCs), macrophages, natural killer cells, and T-lymphocytes. In embodiments, the modified cell lines can also contain the safe-cell system developed by Liang et al., where cell division genes are linked to a suicide gene to prevent runaway teratomas leading to cancers (Liang, et al. “Linking a cell-division gene and a suicide gene to define and improve cell therapy safety.” Nature. Vol. 563, No. 7733, 2018: pp. 701-704.).

In embodiments, methods improve upon the approaches of hypoimmunogenicity of Table 3.

TABLE 3
Expression or increased expression of illustrative
proteins for creating allogenic modified cells.

| | | Illustrative Immunomodulatory |
|---|---|---|
| Gene | Type of protein | Function |
| Ccl2 | Chemokine | Recruitment of activated |
| | | Dendritic Cells |
| PD-L1 | Check Point Inhibitor | Shuts Down Effector T-cell |
| (non- | | Responses |
| activated | | |
| cells | | |
| and/or | | |
| BioNVs) | | |
| FASL | Transmembrane Signal | Induces T-cell Apoptosis |
| H2-M3 | Antigen Presentation - | Inhibits NK cells |
| | MHC-like | |
| SERPINB9 | Inhibitory Chemokine | Blocks granzyme B function |
| | (Serine Protease | |
| | Inhibitor) | |
| Cd47 | Transmembrane SIRP-α | Blocks phagocytosis |
| | Receptor | |
| Cd200 | Type 1 Membrane | Prevents macrophage and |
| | Glycoprotein | granulocyte activation |
| Mfg-e8 | Chemokine (attractant) | Macrophage recruitment to |
| | | anti-inflammatory profile site |

In embodiments, modified cells from which BioNVs are derived are engineered to have knock-outs of one or more of HLA-A, HLA-B, HLA-C, HLA-E, HLA-G, HLA-F, CIITA, IL-6 IL4, IL-10, IL-16, TRAC, TRBC, and/or any combination thereof; and knock-ins of one or more of CCL2, PD-L1, CTLA-4, H2-M3, CD24, CD47 (minus the 3′ UTR region or an alternate 3′ UTR region that does not contain binding sites for the inhibitory microRNAs), MFG-E8, CD200, and/or any combination thereof.

In embodiments, BioNVs are generated from a modified cell with one or more of the modifications of Table 4.

TABLE 4
Illustrative engineered cell expression profile for BioNV
formation for human use (Fife BT and Bluestone JA. “Control
of peripheral T-cell tolerance and autoimmunity via the
CTLA-4 and PD-1 pathways.” Immunol Rev. Vol. 224, 2008:
pp. 166-82.) and (Rong Z, et al. “An effective approach
to prevent immune rejection of human ESC-derived allografts.”
Cell Stem Cell. Vol. 14, No. 1 2014: pp. 121-30.).

| | Knock-ins (inducible or constitutive |
|---|---|
| Knockouts | over-expression) |
| HLA-A | CCL2 |
| HLA-B | PD-L1 and/or CTLA-4 (Fife et al. and |
| | Rong et al.) |
| HLA-C | H2-M3 |
| HLA-E or HLA-G | CD24 or CD47 or CD47 tethered to CD24 |
| (but not both) | |
| HLA-F | MFG-E8 |
| CIITA | CD200 for non-immunomodulatory functions |
| IL-6 | only. These cells will not contain a CD47 tag. |
| IL-4, IL-10, or IL-16 | SerpinB9 |
| (optional) | |
| TRAC | |
| TRBC | |

In embodiments, inactivation/activation of genes is controlled by inducible promoters throughout the differentiation and manufacturing process for BioNVs. In embodiments, disruption of MHC, TCR, and cytokine release syndrome (CRS) genes produce allogeneic iPSCs which are −/−CRS and −/−TCR, leading them to have plasma membranes which exhibit hypoimmunogenic properties upon infusion into a subject. CRS genes implicated in the pathogenesis of CRS include IL-6, IL-10, IFN-γ, monocyte chemoattractant protein 1 (MCP-1), granulocyte-macrophage colony-stimulating factor (GM-CSF), among other cytokines, including tumor necrosis factor (TNF), IL-1, IL-2, IL-2-receptor-α, and IL-8. In embodiments, one or more of these genes is inactivated, e.g., in a cell from which the BioNVs are derived.

In embodiments, BioNVs are formed by disrupting the cell membranes of engineered iPSCs. In embodiments, the hypo-iPSCs are characterized by a B2M−/−, CIITA−/−, CD47+/+, PD1−/− plasma membrane profile and may be used to generate BioNVs. Hypoimmunogenic BioNVs can be generated from the parent iPSC cell line via sonication, adaptive focused acoustics technology, French press, extrusion, serial extrusion, cell lysis by detergent, and electroporation, among other methods. In embodiments, serial extrusion is the method used to generate hypoimmunogenic BioNVs. In embodiments, serial extrusion of iPSCs can produce BioNVs that are HLA1/HLA2 negative (hypoimmunogenic) with tgCD47+, exhibiting PD1 resistance elimination.

In embodiments, genetic engineering of iPSCs includes gene-editing techniques such as CRISPR-based gene editing systems, zinc finger nucleases (ZFNs), transcription activator-like effector nuclease (TALEN), meganucleases, among other gene editing methods, for the purpose of generating allogeneic/hypoimmunogenic iPSCs and/or for targeting agent cassette integration. In embodiments, the genetic engineering of iPSCs can refer to the decrease or ablation of transcription of any genetic element; likewise, genetic engineering of iPSCs can refer to the increase in the expression of or the knock-in of any genetic element, including both endogenous and exogenous genetic elements.

For example, in embodiments, stable cell integration (safe harbor genetic location) in iPSCs can be controlled by implementing a Tet-regulated CRISPRa+targeted 3× transcription factor targeted gRNA system. The CRISPR activation system for three upstream transcription factors can bigger a signal cascade event that enhances the production of VERRs/viral ligands that have replaced endogenous antibody ORFs at designated locus(loci). This system can be ‘tunable’ by including a Tet-regulated promoter, allowing for the ability to vary the concentrations of VERRs/viral ligands on the surface of the cell. Next, stable cell replacement of CDRs and heavy and light antibody regions with VERRs/viral ligands cassettes can be achieved via Cpf-1 directed homology directed repair (HDR). Finally, the stably integrated targeting agent cassette can contain flanking gRNA binding sites which allow the targeting agent binding moiety to be repeatedly swapped or altered for rapid and consistent insertion of a desired sequence.

In embodiments, the allogeneic and hypoimmunogenic properties of the iPSCs can be further improved by inducing overexpression of immunoprotective molecules. For example, and without limitation, overexpression of CD47—among other cell surface integrins—can decrease the kinetics of macrophage depletion of BioNV products from the blood. In embodiments, additionally, allogeneic and hypoimmunogenic properties of iPSCs can be improved by expression of α- and β-phagocytic integrins. In embodiments, overexpression of similar immunogenically protective cell surface markers which signal to leukocytes can be performed as a strategy to increase the half-life of BioNVs post-infusion.

In embodiments, iPSCs are genetically engineered for targeting agent cassette integration. Targeting agent cassette integration can include both integrative and non-integrative transgene insertion. Non-limiting examples of non-integrative transgene insertion include mRNA, non-integrative lentivirus, and endonuclease-targeted methods. Integrative targeting agent cassette insertion methods include stable retroviral vector insertion and transposase-based integration systems. Stable targeting agent cassette transduction can be achieved, for example, using retroviral vectors which can enable iPSCs to maintain the genetic element encoding the targeting agent throughout differentiation, expansion, and activation. In embodiments, clinical-grade, stable transduction of targeting agent cassettes into T cells has been achieved for similar CAR cassettes, for example, in brexucabtagene autoleucel (Tecartus®, Kite Pharma Inc.) and axicabtagene ciloleucel (Yescarta®, Kite Pharma Inc.) using GRV vectors, while tisagenlecleucel (Kymriah®, Novartis International AG) is transduced using a lentiviral vector (Labbé, R. P., et al. “Lentiviral Vectors for T Cell Engineering: Clinical Applications, Bioprocessing and Future Perspectives.” Viruses. Vol. 13, No. 8, 2021: 1-22.).

In embodiments, the concentration of the targeting agent on the surface of the iPSC base cell line, or any downstream differentiated cell (and the resulting BioNVs), can be regulated using a variety of transcription control elements, such as a tetracycline on/off promoter (or similar drug-regulated promoters) to drive the expression of a CRISPR activation/gRNA (CRISPRa) system. The CRISPRa system can then activate the antibody-regulating transcription factors, for example, Drm2, Fr5, and Bxp2, which regulate the expression of an engineered targeting agent cassette that has been integrated at the site of an antibody locus (where the antibody genes have been replaced). Additionally, a similar transcription control element can be provided to control overexpression of genes (e.g., CD47), drive genes controlling differentiation, etc., at defined manufacturing stages.

In embodiments, targeting agent expression is initiated in the modified cells with or without differentiation. In embodiments, targeting agent expression can be performed in iPSCs without differentiation to obtain BioNVs lacking cell surface markers from differentiated cell subsets. Alternatively, iPSCs can be differentiated into a lymphoid or myeloid lineage cell prior to initiating targeting agent expression of cell surface markers from select immune cell types.

Subjects and/or Animals

In embodiments, the subject and/or animal is a mammal, e.g., a human, primate, mouse, rat, guinea pig, dog, cat, horse, cow, pig, rabbit, sheep, or non-human primate. In embodiments, the subject and/or animal is a transgenic animal comprising a fluorescent cell, such as, for example, an RPE cell and/or an immune cell with GFP. In embodiments, the subject and/or animal is a human. In embodiments, the human is a pediatric human, human adult, geriatric human, an infant or child. In other embodiments, the human is referred to as a subject, or a patient.

In embodiments, the method of treatment includes administering to a human who has an age in a range of from about 0 months to about 6 months old, from about 6 months to about 12 months old, from about 12 months to about 18 months old, from about 18 months to about 36 months old, from about 1 year to about 5 years old, from about 5 years to about 10 years old, from about 10 years to about 15 years old, from about 15 years to about 20 years old, from about 20 years to about 25 years old, from about 25 years to about 30 years old, from about 30 years to about 35 years old, from about 35 years to about 40 years old, from about 40 years to about 45 years old, from about 45 years to about 50 years old, from about 50 years to about 55 years old, from about 55 years to about 60 years old, from about 60 years to about 65 years old, from about 65 years to about 70 years old, from about 70 years to about 75 years old, from about 75 years to about 80 years old, from about 80 years to about 85 years old, from about 85 years to about 90 years old, from about 90 years to about 95 years old or from about 95 years to about 100 years old.

In embodiments, the subject is a non-human animal, and therefore the invention pertains to veterinary use. In embodiments, the non-human animal is a household pet, a livestock animal, or a laboratory animal.

In embodiments, sera and/or immune cells are evaluated and/or effected. In embodiments, immune cells include cells of a subject's and/or animal's innate immune system. In embodiments, such cells include, but are not limited to, NK cell, monocyte, DC, B cell, macrophage, CD4+ T cell, and CD8+ T cell. In various embodiments, the invention provides for detecting a presence, detecting an absence, or measuring an amount of viral cDNA or RNA in a sample originating from a subject.

### Kits

The disclosure, in embodiments, provides kits that can simplify the administration of any agent described herein. An exemplary kit of the invention comprises any agent described herein in unit dosage form. In embodiments, the unit dosage form is a container, such as a pre-filled syringe, which can be sterile, containing any agent described herein and a pharmaceutically acceptable carrier, diluent, excipient, or vehicle. In embodiments, the kit further comprises a label or printed instructions instructing the use of any agent described herein. In embodiments, the kit also includes a lid speculum, topical anesthetic, and a cleaning agent for the injection surface. In embodiments, the kit further comprises one or more additional agents described herein.

In aspects, the present invention includes a syringe comprising one or more compositions of the present invention. In embodiments, the syringe is prefilled with a volume of the composition. In embodiments, the syringe is prefilled in a volume of about 1 mL to about 10 mL. In embodiments, the syringe is prefilled in a volume of about 10 mL, about 9 mL, about 8 mL, about 7 mL, about 6 mL, about 5 mL, about 4 mL, about 3 mL, about 2 mL, about 1.9 mL, about 1.8 mL, about 1.7 mL, about 1.6 mL, about 1.5 mL, about 1.4 mL, about 1.3 mL, about 1.2 mL, about 1.1 mL, or about 1.0 mL or less of the composition.

In embodiments, the syringe comprises a composition having a shelf stability ranging from about 1 hour to about 1 week. In embodiments, the syringe comprises a composition having a shelf stability of at least about 12 hours, about 24 hours, about 36 hours, about 48 hours, or about 72 hours when stored at a temperature ranging from about −85° C. to about 25° C. In embodiments, the syringe comprises a composition having a shelf stability of at least about 12 hours, about 24 hours, about 36 hours, about 48 hours, or about 72 hours when stored at a temperature ranging from about 15° C. to about 25° C.

In embodiments, the storage temperature is about −80° C. In embodiments, the storage temperature is about −20° C. In embodiments, the storage temperature is about 4° C. In embodiments, the storage temperature is about 21° C. In embodiments, the kit includes lyophilized BioNVs.

In one embodiment, the kit comprises a container containing a composition comprising BioNVs of the present invention, a therapeutically effective amount of an additional therapeutic agent, such those described herein, and instructions for use.

### Definitions

The following definitions are used in connection with the invention disclosed herein. Unless defined otherwise, all technical and scientific terms used herein have the same meaning as commonly understood to one of skill in the art to which this invention belongs.

An “effective amount,” or “therapeutically effective amount,” is an amount that is effective for treating, preventing, or ameliorating HIV or a clinical symptom of HIV/AIDS, such as those described herein, or an amount that is intended to reduce the number of virally-infected cells, including latently infected cells, and/or reduce the amount of detectable viral DNA (or cDNA), RNA, and/or protein in a subject.

As used herein, “a,” “an,” or “the” can mean one or more than one.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, and may alternatively be described using alternative terms such as “consisting of” or “consisting essentially of.”

In embodiments, “BioNVs,” as referred to herein, are allogeneic, hypoimmunogenic biomimetic nanovesicles which comprise at least one surface-oriented, membrane-embedded targeting agent. In embodiments, “nanovesicles (NVs),” as referred to herein, are lipid-bound vesicles on the order of about 10 nm to about 1200 nm in size which encapsulate an aqueous core. In embodiments, lipid-bound NVs can form using lipid monolayers, lipid bilayers, or maintain multilamellar forms. In embodiments, BioNV refers to biologically-derived nano-sized vesicles that can have designed biological functionalization. In embodiments, BioNVs are “biomimetic” in that they are derived from endogenous cellular material; more specifically, they substantially recapitulate plasma membrane material found in cells. In embodiments, the cells from which BioNVs originate can include stem cells of any kind, including cell types differentiated from said stem cells. In embodiments, BioNVs are substantially free of encapsulated cellular debris including nucleic acid, organelles, or organelle parts. In embodiments, BioNVs are characterized as having one or more, 2 or more, 3 or more, 4 or more, 5 or more, or 6 or more of the following:
a. being about 10 nm to about 1200 nm in size;
b. having a total volume of about 500 nm³ to about 5 μm³ (assuming spherical shape);
c. having a content of at least one phospholipid and cholesterol;
d. having a surface membrane having one or more of CD34, CCL21, PD-L1 (in BioNVs derived from non-activated cell sources), FasL, SerpinB9, H2-M3, CD47, CTLA-4, CD24, CD200, MFG-E8, NCAM, and/or α-phagocytic integrin, or a chimera of any one or more thereof; having a surface membrane substantially lacking T cell receptor components (TRAC and/or TRBC), MHC class I components, and/or MHC class II components, lacking proteins of one or more of HLA-A, HLA-B, HLA-C, HLA-E or HLA-G (but not both of HLA-E and HLA-G), HLA-F, and/or CIITA, substantially lacking proteins inside the vesicle of one or more of ILA, IL-6, IL-10, and/or IL-16;
e. having a membrane-embedded targeting agent comprising a target-binding moiety which can include one or more of a CAR, VERR, viral ligand/receptor, an antibody or antibody format selected from monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, fusion protein comprising the antigen-binding portion of an antibody, Bispecific T cell Engagers (BiTE), or by a variable heavy chain IgG fragment V_{H}H or V_{NAR} or through a T-Cell Receptor (TCR);
f. being capable of adsorbing and/or encapsulating a payload content of one or more checkpoint inhibiting agents, gene editing payloads, gRNAs, small RNAs, small molecule inhibitors, biologics, fluorescent proteins, fusion proteins, among other therapeutic payloads, and/or any combination thereof; and
g. being capable of not causing a deleterious immune reaction in subjects.

In embodiments, “induced pluripotent stem cells,” “iPSCs,” or “reprogrammed induced pluripotent stem cells,” are stem cells that originate from differentiated cells and are reprogrammed back into an embryonic-like pluripotent state. iPSCs can generally propagate indefinitely and become any cell type of the organism they originate.

In embodiments, “allogeneic,” as used herein, refers to biological material, tissues, or cells, which are genetically dissimilar and originally immunological incompatible, despite originating from the same species. Allogeneic BioNVs, for example, are material that originates from a first subject (iPSC donor) and can be provided to any number of distinct subjects who are not genetically identical.

In embodiments, “hypoimmunogenic” or “hypoimmune,” as used herein in reference to a modified cell and/or BioNV, refers to a reduced capacity to generate an immunological response. iPSCs and BioNVs can be hypoimmunogenic due to reduced or ablated expression of immunogenic cell surface proteins, such as T cell receptor (TCR) proteins, cytokine response syndrome proteins, MHC class I or II proteins, etc. iPSCs and BioNVs can be hypoimmunogenic due to expression of one or more immunoprotective cell surface proteins, such as CD47 and α-phagocytic integrins. BioNVs can be hypoimmunogenic due to not triggering CRS in a subject and/or not inducing HLA incompatibility.

In embodiments, “knocking-out,” silencing, “inactivating,” “disrupting,” or “blocking,” and their equivalencies, with respect to transcription, gene, or protein expression, refers to an amount of transcription, gene or protein expression which is reduced from a normal state or less than the wild-type state in a particular cell subset. The reduction can be significant so that no gene expression occurs, or a negligible amount of protein expression occurs.

In embodiments, “overexpression,” as used herein, refers to an amount of transcription, gene or protein expression which is increased from a normal state or more than the wild-type state in a particular cell subset.

### EXAMPLES

### Example 1: Evaluating CRISPR-Packaged BioNV Gene Editing-Based Approach for Partial, Targeted Excision of HIV-1 Genomes

Efficient excision of partial proviral genomes can occur in vitro and in vivo to achieve viral deactivation while allowing remnant peptide expression for MHC presentation. Silencing of Nef transcripts or the knockout of Nef genes can replenish MHC complex exposure on the surface of the infected cell to a concentration sufficient to alert/train the immune system for elimination of HIV-infected cell populations. Cells with replenished MHC complexes can be cleared by a CD8 T cell response. The RNA interference silencing effect of Nef can spread from cell to cell, exposing MHC complexes on the surface of cells that were not targeted by the original BioNV treatment.

In vitro Evaluation: for in vitro experiments, primary CD4+ T cells can be used. Alternatively, cell lines engineered to express HIV-1 sequences can be used to offer convenience and specific modifications for targeted experiments.

TZM-bl cells: these cells are derived from HeLa cells and contain integrated copies of the luciferase and β-galactosidase genes under the control of the HIV-1 long terminal repeat (LTR) promoter. They can be used for HIV-1 infectivity assays and studying viral entry.

Jurkat cells: this T lymphocyte cell line is frequently used in HIV-1 research. Various subclones of Jurkat cells can be engineered to express HIV-1 genes or reporter genes driven by HIV-1 promoters, allowing the study of viral replication and gene expression.

SupT1 cells: these T lymphocyte cells have been modified to express high levels of the CD4 receptor, making them highly susceptible to HIV-1 infection. They can be used to study viral replication and the effects of potential antiviral therapies.

U87MG-CD4-CXCR4 cells: these human glioblastoma cells have been engineered to express both CD4 and CXCR4, the primary coreceptors for HIV-1 entry. They can be used for studying HIV-1 entry and infection mechanisms.

MT-4 cells: this T lymphoblastoid cell line is highly permissive to HIV-1 infection and supports viral replication. MT-4 cells can be used to study viral replication, viral tropism, and drug susceptibility.

In vivo Evaluation: for in vivo experiments, humanized mice (such as BLT and CD34+ NSG mice) can be used as model to evaluate the ability of BioNVs to target HIV-infected cells and reduce the populations of HIV infected cells. These mice are genetically engineered to express human genes necessary for HIV-1 infection, enabling the establishment of viral latency. Another model, NOD/SCID mice, lack key immune system components, making them susceptible to HIV-1 infection and latency development. Rag2−/− and γc−/− mice, deficient in genes essential for T cell development, offer similar susceptibility. Additionally, BLT mice, featuring human bone marrow, liver, and thymus tissue, can provide a more human-like immune system for comprehensive studies.

In vivo delivery of the CRISPR-Cas9 system to latent HIV-1-infected mice can be achieved through techniques such as intra-peritoneal injection and/or tail vein injection to ensure effective delivery to the target cells within the animal model. A dosage escalation scheme can be provided to delineate a dose-responsive effect and to evaluate toxicity pharmacokinetic (PK) parameters. One or more latency reversal agents (LRAs) can also be provided at designated time points to initiate active HIV infection from latency, where the immunological responses can be evaluated. Animals treated with the CRISPR-packaged BioNV gene editing-based approach would show reduced HIV-infected cellular populations and increased adaptive immunological responses to one or more HIV antigens in comparison to control animals (e.g., not treated), or in comparison to control animals treated with combined antiretroviral therapy (cART) alone. Control animals may also exhibit one or more symptoms (or increased severity of symptoms) in comparison to treated animals (or animals treated with the gene editing system and ART), and may potentially exhibit reduced survival in comparison to treated animals (or animals treated with the gene editing system and ART).

One of the best non-human primate models to study Simian Immunodeficiency Virus (SIV) is the rhesus macaque. Rhesus macaques are an animals model that is more closely related to humans, and can be infected with SIV, which is the simian version of HIV. Rhesus macaques develop a disease that is similar to AIDS in humans, and they can be used to study the pathogenesis of SIV infection, as well as the development of vaccines and treatments. In particular, we can study rhesus macaques infected with SHIV.C.CH848 (SHIVC) and treated with combined antiretroviral therapy (cART). Similar to HIV and SIV infection, SHIV.C.CH848 infection establishes viral reservoirs in CD4+ T cells and myeloid cells. These reservoirs lead to productive infection and depletion of CD4+ T cells in systemic and lymphoid tissues throughout the course of SHIV infection. As a result, the integrated latent pool of SHIVC virus is more stable and more closely mimics the stable latent HIV pool in humans.

gRNA Design: to specifically target the latent HIV-1 genome, e.g., as described herein, guide RNAs (gRNAs) can be designed. These gRNAs can be designed to selectively recognize conserved sequences with little or no predicted off-targeting sequences related to CRISPR-Cas9 and other CRISPR endonucleases. Once the gRNAs are designed, their activity can be confirmed using in vitro and in vivo models and deep genomic sequencing will be utilized to determine the degree of potential off targeting.

TABLE 5
Illustrative examples of gRNAs used for targeting HIV sequences for partial genomic excision.
nef (AAC82597.1)|Cas9

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| ATTAGCAGAACTACACACCAGGG | AF033819.3: 8710 | + | 40 | 1 | 0 | 0 | 0 | 0 | 74.78 |
| (SEQ ID NO: 1) | | | | | | | | | |
| ATTGGTCTTAAAGGTACCTGAGG | AF033819.3: 8556 | − | 40 | 0 | 0 | 0 | 0 | 0 | 68.73 |
| (SEQ ID NO: 2) | | | | | | | | | |
| CAGGGAAGTAGCCTTGTGTGTGG | AF033819.3: 8687 | − | 55 | 0 | 0 | 0 | 0 | 0 | 66.2 |
| (SEQ ID NO: 3) | | | | | | | | | |
| TGCCTGGCTAGAAGCACAAGAGG | AF033819.3: 8507 | + | 55 | 2 | 0 | 0 | 0 | 0 | 66.67 |
| (SEQ ID NO: 4) | | | | | | | | | |
| GGATTTTGCTATAAGATGGGTGG | AF033819.3: 8328 | + | 40 | 0 | 0 | 0 | 0 | 0 | 64.06 |
| (SEQ ID NO: 5) | | | | | | | | | |
| CTGTGGATCTACCACACACAAGG | AF033819.3: 8676 | + | 50 | 2 | 0 | 0 | 0 | 0 | 65.44 |
| (SEQ ID NO: 6) | | | | | | | | | |
| GACAAGATATCCTTGATCTGTGG | AF033819.3: 8659 | + | 40 | 1 | 0 | 0 | 0 | 0 | 63.82 |
| (SEQ ID NO: 7) | | | | | | | | | |
| GCACAAGAGGAGGAGGAGGTGGG | AF033819.3: 8520 | + | 60 | 0 | 0 | 0 | 0 | 0 | 61.92 |
| (SEQ ID NO: 8) | | | | | | | | | |
| CACTTTTTAAAAGAAAAGGGGGG | AF033819.3: 8607 | + | 30 | 0 | 0 | 0 | 0 | 0 | 61.16 |
| (SEQ ID NO: 9) | | | | | | | | | |
| CAAAAAGTAGTGTGATTGGATGG | AF033819.3: 8359 | + | 35 | 0 | 0 | 0 | 0 | 0 | 60.45 |
| (SEQ ID NO: 10) | | | | | | | | | |
| GCTATAAGATGGGTGGCAAGTGG | AF033819.3: 8335 | + | 50 | 0 | 0 | 0 | 0 | 0 | 59.72 |
| (SEQ ID NO: 11) | | | | | | | | | |
| CTGGCTAGAAGCACAAGAGGAGG | AF033819.3: 8510 | + | 55 | 1 | 0 | 0 | 0 | 0 | 59.91 |
| (SEQ ID NO: 12) | | | | | | | | | |
| GATTAGCAGAACTACACACCAGG | AF033819.3: 8709 | + | 45 | 1 | 0 | 0 | 0 | 0 | 59.79 |
| (SEQ ID NO: 13) | | | | | | | | | |
| GAGCCAGCAGCAGATAGGGTGGG | AF033819.3: 8412 | + | 60 | 1 | 0 | 0 | 0 | 0 | 58.96 |
| (SEQ ID NO: 14) | | | | | | | | | |
| CCACTTTTTAAAAGAAAAGGGGG | AF033819.3: 8606 | + | 30 | 0 | 0 | 0 | 0 | 0 | 57.12 |
| (SEQ ID NO: 15) | | | | | | | | | |
| AAGAAAAGGGGGGACTGGAAGGG | AF033819.3: 8617 | + | 50 | 2 | 0 | 0 | 0 | 0 | 58.02 |
| (SEQ ID NO: 16) | | | | | | | | | |
| TGTGGTAGATCCACAGATCAAGG | AF033819.3: 8669 | − | 45 | 1 | 0 | 0 | 0 | 0 | 56.63 |
| (SEQ ID NO: 17) | | | | | | | | | |
| GATTGGATGGCCTACTGTAAGGG | AF033819.3: 8372 | + | 45 | 1 | 0 | 0 | 0 | 0 | 56.13 |
| (SEQ ID NO: 18) | | | | | | | | | |
| TCTCGAGACCTGGAAAAACATGG | AF033819.3: 8442 | + | 45 | 0 | 0 | 0 | 0 | 0 | 54.75 |
| (SEQ ID NO: 19) | | | | | | | | | |
| ACAGCTGCCTTGTAAGTCATTGG | AF033819.3: 8574 | − | 45 | 1 | 0 | 0 | 0 | 0 | 55.3 |
| (SEQ ID NO: 20) | | | | | | | | | |

nef (AAC82597.1)|Cpf1 CasX

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| TTCCAGGTCTCGAGATGCTGCTCCCACC | AF033819.3: 8429 | − | 62 | 0 | 0 | 0 | 0 | 0 | — |
| (SEQ ID NO: 21) | | | | | | | | | |
| TTCTAGCCAGGCACAAGCAGCATTGGTA | AF033819.3: 8492 | − | 54 | 0 | 0 | 0 | 0 | 0 | — |
| (SEQ ID NO: 22) | | | | | | | | | |
| TTCTTTTAAAAAGTGGCTAAGATCTACA | AF033819.3: 8594 | − | 29 | 0 | 0 | 0 | 0 | 0 | — |
| (SEQ ID NO: 23) | | | | | | | | | |
| TTCCAGTCCCCCCTTTTTTTTAAAAAG | AF033819.3: 8609 | − | 38 | 0 | 0 | 0 | 0 | 0 | — |
| (SEQ ID NO: 24) | | | | | | | | | |
| TTCTTTGGGAGTGAATTAGCCCTTCCAG | AF033819.3: 8631 | − | 50 | 0 | 0 | 0 | 0 | 0 | — |
| (SEQ ID NO: 25) | | | | | | | | | |
| TTCACTCCCAAAGAAGACAAGATATCCT | AF033819.3: 8644 | + | 42 | 0 | 0 | 0 | 0 | 0 | — |
| (SEQ ID NO: 26) | | | | | | | | | |
| TTCCCTGATTAGCAGAACTACACACCAG | AF033819.3: 8703 | + | 46 | 0 | 0 | 0 | 0 | 0 | — |
| (SEQ ID NO: 27) | | | | | | | | | |
| TTCCCTTACAGTAGGCCATCCAATCACA | AF033819.3: 8369 | − | 46 | 0 | 0 | 0 | 0 | 0 | — |
| (SEQ ID NO: 28) | | | | | | | | | |
| TTCTTTCCCTTACAGTAGGCCATCCAAT | AF033819.3: 8373 | − | 46 | 0 | 0 | 0 | 0 | 0 | — |
| (SEQ ID NO: 29) | | | | | | | | | |
| TTCCAGTCACACCTCAGGTACCTTTAAG | AF033819.3: 8545 | + | 46 | 1 | 0 | 0 | 0 | 0 | — |
| (SEQ ID NO: 30) | | | | | | | | | |
| TTCTGCTAATCAGGGAAGTAGCCTTGTG | AF033819.3: 8692 | 1 | 50 | 1 | 0 | 0 | 0 | 0 | — |
| (SEQ ID NO: 31) | | | | | | | | | |

pol (AAC82598)|Cas9

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| GCATGGGTACCAGCACACAAAGG | AF033819.3: 3695 | + | 55 | 0 | 0 | 0 | 0 | 0 | 75.67 |
| (SEQ ID NO: 32) | | | | | | | | | |
| GTATAAACAATGAGACACCAGGG | AF033819.3: 2496 | + | 35 | 0 | 0 | 0 | 0 | 0 | 73.97 |
| (SEQ ID NO: 33) | | | | | | | | | |
| CTCCCACTCAGGAATCCAGGTGG | AF033819.3: 3318 | − | 60 | 1 | 0 | 0 | 0 | 0 | 73.23 |
| (SEQ ID NO: 34) | | | | | | | | | |
| TGATGTAAAACAATTAACAGAGG | AF033819.3: 3184 | + | 25 | 0 | 0 | 0 | 0 | 0 | 71.54 |
| (SEQ ID NO: 35) | | | | | | | | | |
| GGGCAAGTCAGATTTACCCAGGG | AF033819.3: 2892 | + | 50 | 0 | 0 | 0 | 0 | 0 | 69.51 |
| (SEQ ID NO: 36) | | | | | | | | | |
| CGTTGCCAAAGAGTGACCTGAGG | AF033819.3: 1799 | − | 55 | 0 | 0 | 0 | 0 | 0 | 68.43 |
| (SEQ ID NO: 37) | | | | | | | | | |
| TCGTCACAATAAAGATAGGGGGG | AF033819.3: 1827 | + | 40 | 0 | 0 | 0 | 0 | 0 | 68.43 |
| (SEQ ID NO: 38) | | | | | | | | | |
| GATGGCAGGTGATGATTGTGTGG | AF033819.3: 4597 | + | 50 | 0 | 0 | 0 | 0 | 0 | 67.66 |
| (SEQ ID NO: 39) | | | | | | | | | |
| AGGAAACATGGGAAACATGGTGG | AF033819.3: 3279 | + | 45 | 1 | 0 | 0 | 0 | 0 | 68.42 |
| (SEQ ID NO: 40) | | | | | | | | | |
| GTTGCCAAAGAGTGACCTGAGGG | AF033819.3: 1798 | − | 50 | 0 | 0 | 0 | 0 | 0 | 66.99 |
| (SEQ ID NO: 41) | | | | | | | | | |
| GGTGATCCTTTCCATCCCTGTGG | AF033819.3: 2543 | − | 55 | 0 | 0 | 0 | 0 | 0 | 66.68 |
| (SEQ ID NO: 42) | | | | | | | | | |
| ATTCTAAAAGAACCAGTACATGG | AF033819.3: 3020 | + | 30 | 0 | 0 | 0 | 0 | 0 | 66.66 |
| (SEQ ID NO: 43) | | | | | | | | | |
| TGGAAACCAAAAATGATAGGGGG | AF033819.3: 1922 | + | 35 | 0 | 0 | 0 | 0 | 0 | 66.13 |
| (SEQ ID NO: 44) | | | | | | | | | |
| TATCGGCTCCTGCTTCTGAGGGG | AF033819.3: 1750 | − | 55 | 1 | 0 | 0 | 0 | 0 | 66.67 |
| (SEQ ID NO: 45) | | | | | | | | | |
| ACAGAAAGCATAGTAATATGGGG | AF033819.3: 3224 | + | 30 | 0 | 0 | 0 | 0 | 0 | 65.53 |
| (SEQ ID NO: 46) | | | | | | | | | |
| AAGCCACCTGGATTCCTGAGTGG | AF033819.3: 3315 | + | 55 | 1 | 0 | 0 | 0 | 0 | 66.46 |
| (SEQ ID NO: 47) | | | | | | | | | |
| ACAGGATGAGGATTAGAACATGG | AF033819.3: 4627 | + | 40 | 0 | 0 | 0 | 0 | 0 | 65.25 |
| (SEQ ID NO: 48) | | | | | | | | | |
| AGAAACCTTCTATGTAGATGGGG | AF033819.3: 3406 | + | 35 | 0 | 0 | 0 | 0 | 0 | 65.04 |
| (SEQ ID NO: 49) | | | | | | | | | |
| CAGATACTCATAGAAATCTGTGG | AF033819.3: 1979 | + | 35 | 1 | 0 | 0 | 0 | 0 | 66.02 |
| (SEQ ID NO: 50) | | | | | | | | | |
| ACAATTTTAAAAGAAAAGGGGGG | AF033819.3: 4323 | + | 25 | 0 | 0 | 0 | 0 | 0 | 64.73 |
| (SEQ ID NO: 51) | | | | | | | | | |

pol (AAC82598.2)|Cpf1 CasX

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| TTCTAAGTCAGATCCTACATACAAATCA | AF033819.3: 2650 | − | 33 | 0 | 0 | 0 | 0 | 0 | — |
| (SEQ ID NO: 52) | | | | | | | | | |
| TTCTATGCTGCCCTATTTCTAAGTCAGA | AF033819.3: 2666 | − | 42 | 0 | 0 | 0 | 0 | 0 | — |
| (SEQ ID NO: 53) | | | | | | | | | |
| TTCTGATGTTTTTTGTCTGGTGTGGTAA | AF033819.3: 2736 | − | 38 | 0 | 0 | 0 | 0 | 0 | — |
| (SEQ ID NO: 54) | | | | | | | | | |
| TTCTTTCTGATGTTTTTTGTCTGGTGTG | AF033819.3: 2740 | − | 38 | 0 | 0 | 0 | 0 | 0 | — |
| (SEQ ID NO: 55) | | | | | | | | | |
| TTCCCTAAAAAATTAGCCTGTCTCTCAG | AF033819.3: 1615 | − | 38 | 0 | 0 | 0 | 0 | 0 | — |
| (SEQ ID NO: 56) | | | | | | | | | |
| TTCCCCACTAACTTCTGTATGTCATTGA | AF033819.3: 2856 | − | 38 | 0 | 0 | 0 | 0 | 0 | — |
| (SEQ ID NO: 57) | | | | | | | | | |
| TTCAATTTCCCCACTAACTTCTGTATGT | AF033819.3: 2862 | − | 38 | 0 | 0 | 0 | 0 | 0 | — |
| (SEQ ID NO: 58) | | | | | | | | | |
| TTCTGAGGGGGAGTTGTTGTCTCTACCC | AF033819.3: 1732 | − | 58 | 0 | 0 | 0 | 0 | 0 | — |
| (SEQ ID NO: 59) | | | | | | | | | |
| TTCTGTTAGTGCTTTGGTTCCTCTAAGG | AF033819.3: 2941 | − | 46 | 0 | 0 | 0 | 0 | 0 | — |
| (SEQ ID NO: 60) | | | | | | | | | |
| TTCTGTTAGTGGTATTACTTCTGTTAGT | AF033819.3: 2959 | − | 33 | 0 | 0 | 0 | 0 | 0 | — |
| (SEQ ID NO: 61) | | | | | | | | | |
| TTCTTCTGTTAGTGGTATTACTTCTGTT | AF033819.3: 2962 | − | 33 | 0 | 0 | 0 | 0 | 0 | — |
| (SEQ ID NO: 62) | | | | | | | | | |
| TTCTAGCTCTGCTTCTTCTGTTAGTGGT | AF033819.3: 2974 | − | 46 | 0 | 0 | 0 | 0 | 0 | — |
| (SEQ ID NO: 63) | | | | | | | | | |
| TTCTGCCAGTTCTAGCTCTGCTTCTTCT | AF033819.3: 2983 | − | 50 | 0 | 0 | 0 | 0 | 0 | — |
| (SEQ ID NO: 64) | | | | | | | | | |
| TTCTTTTAGAATCTCTCTGTTTTCTGCC | AF033819.3: 3004 | − | 38 | 0 | 0 | 0 | 0 | 0 | — |
| (SEQ ID NO: 65) | | | | | | | | | |
| TTCTAAAAGAACCAGTACATGGAGTGTA | AF033819.3: 3021 | + | 38 | 0 | 0 | 0 | 0 | 0 | — |
| (SEQ ID NO: 66) | | | | | | | | | |
| TTCTGCTATTAAGTCTTTTGATGGGTCA | AF033819.3: 3052 | − | 38 | 0 | 0 | O | 0 | 0 | — |
| (SEQ ID NO: 67) | | | | | | | | | |
| TTCTGTATTTCTGCTATTAAGTCTTTTG | AF033819.3: 3060 | − | 29 | 0 | 0 | 0 | 0 | 0 | — |
| (SEQ ID NO: 68) | | | | | | | | | |
| TTCCTTGTCTATCGGCTCCTGCTTCTGA | AF033819.3: 1754 | − | 50 | 0 | 0 | 0 | 0 | 0 | — |
| (SEQ ID NO: 69) | | | | | | | | | |
| TTCAGATTTTTAAATGGCTCTTGATAAA | AF033819.3: 3117 | − | 25 | 0 | 0 | 0 | 0 | 0 | — |
| (SEQ ID NO: 70) | | | | | | | | | |
| TTCCTGTTTTCAGATTTTTAAATGGCTC | AF033819.3: 3125 | 1 | 29 | 0 | 0 | 0 | 0 | 0 | — |
| (SEQ ID NO: 71) | | | | | | | | | |

rev (AAC82592.1)|Cas9

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| ACTTACTCTTGATTGTAACGAGG | AF033819.3: 8088 | + | 35 | 0 | 0 | 0 | 0 | 0 | 70.61 |
| (SEQ ID NO: 72) | | | | | | | | | |
| TCAAGAGTAAGTCTCTCAAGCGG | AF033819.3: 8077 | − | 40 | 2 | 0 | 0 | 0 | 0 | 72.53 |
| (SEQ ID NO: 73) | | | | | | | | | |
| GAAGGAATAGAAGAAGAAGGTGG | AF033819.3: 7961 | + | 40 | 0 | 0 | 0 | 0 | 0 | 68.86 |
| (SEQ ID NO: 74) | | | | | | | | | |
| AGATCCATTCGATTAGTGAACGG | AF033819.3: 8000 | + | 35 | 0 | 0 | 0 | 0 | 0 | 65.88 |
| (SEQ ID NO: 75) | | | | | | | | | |
| ACCCACCTCCCAACCCCGAGGGG | AF033819.3: 7925 | + | 70 | 0 | 0 | 0 | 0 | 0 | 65.55 |
| (SEQ ID NO: 76) | | | | | | | | | |
| ATCGTCCCAGATAAGTGCCAAGG | AF033819.3: 8025 | − | 50 | 0 | 0 | 0 | 0 | 0 | 64.54 |
| (SEQ ID NO: 77) | | | | | | | | | |
| AGACCCACCTCCCAACCCCGAGG | AF033819.3: 7923 | + | 70 | 0 | 0 | 0 | 0 | 0 | 64.06 |
| (SEQ ID NO: 78) | | | | | | | | | |
| AGAGTAAGTCTCTCAAGCGGTGG | AF033819.3: 8074 | − | 50 | 1 | 0 | 0 | 0 | 0 | 63.47 |
| (SEQ ID NO: 79) | | | | | | | | | |
| TGGAACTTCTGGGACGCAGGGGG | AF033819.3: 8115 | + | 60 | 1 | 0 | 0 | 0 | 0 | 62.08 |
| (SEQ ID NO: 80) | | | | | | | | | |
| GGGCCTGTCGGGTCCCCTCGGGG | AF033819.3: 7938 | − | 80 | 2 | 0 | 0 | 0 | 0 | 61.03 |
| (SEQ ID NO: 81) | | | | | | | | | |
| CTTGATTGTAACGAGGATTGTGG | AF033819.3: 8095 | + | 40 | 0 | 0 | 0 | 0 | 0 | 58.74 |
| (SEQ ID NO: 82) | | | | | | | | | |
| GTGGAACTTCTGGGACGCAGGGG | AF033819.3: 8114 | + | 60 | 1 | 0 | 0 | 0 | 0 | 59.17 |
| (SEQ ID NO: 83) | | | | | | | | | |
| TCAAGCGGTGGTAGCTGAAGAGG | AF033819.3: 8062 | − | 55 | 0 | 0 | 0 | 0 | 0 | 57.5 |
| (SEQ ID NO: 84) | | | | | | | | | |
| GGATCCGTTCACTAATCGAATGG | AF033819.3: 8004 | − | 45 | 1 | 0 | 0 | 0 | 0 | 58 |
| (SEQ ID NO: 85) | | | | | | | | | |
| GTTCCTGACTCCAATACTGTAGG | AF033819.3: 8167 | − | 45 | 0 | 0 | 0 | 0 | 0 | 56.94 |
| (SEQ ID NO: 86) | | | | | | | | | |
| GGTGGAATCTCCTACAGTATTGG | AF033819.3: 8157 | + | 45 | 0 | 0 | 0 | 0 | 0 | 56.65 |
| (SEQ ID NO: 87) | | | | | | | | | |
| GACCCACCTCCCAACCCCGAGGG | AF033819.3: 7924 | + | 70 | 0 | 0 | 0 | 0 | 0 | 56.4 |
| (SEQ ID NO: 88) | | | | | | | | | |
| AGGGGACCCGACAGGCCCGAAGG | AF033819.3: 7943 | + | 75 | 0 | 0 | 0 | 0 | 0 | 56.72 |
| (SEQ ID NO: 89) | | | | | | | | | |
| CCCGAAGGAATAGAAGAAGAAGG | AF033819.3: 7958 | + | 45 | 0 | 0 | 0 | 0 | 0 | 55.43 |
| (SEQ ID NO: 90) | | | | | | | | | |
| GGGAAGCCCTCAAATATTGGTGG | AF033819.3: 8139 | + | 50 | 1 | 0 | 0 | 0 | 0 | 55.79 |
| (SEQ ID NO: 91) | | | | | | | | | |

rev (AAC82592.1)|Cpf1 CasX

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| TTCAGCTACCACCGCTTGAGAGACTTAC | AF033819.3: 8066 | + | 54 | 0 | 0 | 0 | 0 | 0 | — |
| (SEQ ID NO: 92) | | | | | | | | | |
| TTCCACAATCCTCGTTACAATCAAGAGT | AF033819.3: 8092 | − | 38 | 0 | 0 | 0 | 0 | 0 | — |
| (SEQ ID NO: 93) | | | | | | | | | |
| TTCCCACCCCCTGCGTCCCAGAAGTTCC | AF033819.3: 8116 | − | 67 | 0 | 0 | 0 | 0 | 0 | — |
| (SEQ ID NO: 94) | | | | | | | | | |
| TTCCACCAATATTTGAGGGCTTCCCACC | AF033819.3: 8136 | − | 50 | 0 | 0 | 0 | 0 | 0 | — |
| (SEQ ID NO: 95) | | | | | | | | | |
| TTCTTTAGTTCCTGACTCCAATACTGTA | AF033819.3: 8169 | − | 38 | 0 | 0 | 0 | 0 | 0 | — |
| (SEQ ID NO: 96) | | | | | | | | | |
| TTCGGGCCTGTCGGGTCCCCTCGGGGTT | AF033819.3: 7936 | − | 75 | 0 | 0 | 0 | 0 | 0 | — |
| (SEQ ID NO: 97) | | | | | | | | | |
| TTCCTTCGGGCCTGTCGGGTCCCCTCGG | AF033819.3: 7940 | − | 75 | 0 | 0 | 0 | 0 | 0 | — |
| (SEQ ID NO: 98) | | | | | | | | | |
| TTCTATTCCTTCGGGCCTGTCGGGTCCC | AF033819.3: 7945 | − | 67 | 0 | 0 | 0 | 0 | 0 | — |
| (SEQ ID NO: 99) | | | | | | | | | |
| TTCTTCTATTCCTTCGGGCCTGTCGGGT | AF033819.3: 7948 | − | 58 | 0 | 0 | 0 | 0 | 0 | — |
| (SEQ ID NO: 100) | | | | | | | | | |
| TTCTTCTTCTATTCCTTCGGGCCTGTCG | AF033819.3: 7951 | − | 54 | 0 | 0 | 0 | 0 | 0 | — |
| (SEQ ID NO: 101) | | | | | | | | | |
| TTCACCATTATCGTTTCAGACCCACCTC | AF033819.3: 7906 | + | 50 | 0 | 0 | 0 | 0 | 0 | — |
| (SEQ ID NO: 102) | | | | | | | | | |
| TTCACTAATCGAATGGATCTGTCTCTGT | AF033819.3: 7992 | − | 42 | 0 | 0 | 0 | 0 | 0 | — |
| (SEQ ID NO: 103) | | | | | | | | | |
| TTCGATTAGTGAACGGATCCTTGGCACT | AF033819.3: 8007 | + | 46 | 1 | 0 | 0 | 0 | 0 | — |
| (SEQ ID NO: 104) | | | | | | | | | |
| TTCTGGGACGCAGGGGGTGGGAAGCCCT | AF033819.3: 8121 | + | 75 | 1 | 0 | 0 | 0 | 0 | — |
| (SEQ ID NO: 105) | | | | | | | | | |
| TTCAGACCCACCTCCCAACCCCGAGGGG | AF033819.3: 7920 | + | 75 | 2 | 0 | 0 | 0 | 0 | — |
| (SEQ ID NO: 106) | | | | | | | | | |
| TTCCTGACTCCAATACTGTAGGAGATTC | AF033819.3: 8161 | − | 42 | 2 | 0 | 0 | 0 | 0 | — |
| (SEQ ID NO: 107) | | | | | | | | | |

gag (AAC82593.1)|Cas9

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| GCTACCATAATGATGCAGAGAGG | AF033819.3: 1455 | + | 45 | 0 | 0 | 0 | 0 | 0 | 74.29 |
| (SEQ ID NO: 108) | | | | | | | | | |
| AAACACCATGCTAAACACAGTGG | AF033819.3: 887 | + | 40 | 0 | 0 | 0 | 0 | 0 | 73.61 |
| (SEQ ID NO: 109) | | | | | | | | | |
| AGCATTCTGGACATAAGACAAGG | AF033819.3: 1176 | + | 40 | 1 | 0 | 0 | 0 | 0 | 73.87 |
| (SEQ ID NO: 110) | | | | | | | | | |
| GAGAGCGTCAGTATTAAGCGGGG | AF033819.3: 344 | + | 50 | 0 | 0 | 0 | 0 | 0 | 72.67 |
| (SEQ ID NO: 111) | | | | | | | | | |
| GTTAAAAGAGACCATCAATGAGG | AF033819.3: 935 | + | 35 | 0 | 0 | 0 | 0 | 0 | 71.7 |
| (SEQ ID NO: 112) | | | | | | | | | |
| GGCCAGATGAGAGAACCAAGGGG | AF033819.3: 1011 | + | 55 | 0 | 0 | 0 | 0 | 0 | 71.46 |
| (SEQ ID NO: 113) | | | | | | | | | |
| AAAATTCGGTTAAGGCCAGGGGG | AF033819.3: 387 | + | 45 | 0 | 0 | 0 | 0 | 0 | 70.42 |
| (SEQ ID NO: 114) | | | | | | | | | |
| CTATAGTGCAGAACATCCAGGGG | AF033819.3: 733 | + | 45 | 0 | 0 | 0 | 0 | 0 | 69.31 |
| (SEQ ID NO: 115) | | | | | | | | | |
| AGAGCGTCAGTATTAAGCGGGGG | AF033819.3: 345 | + | 50 | 0 | 0 | 0 | 0 | 0 | 68.54 |
| (SEQ ID NO: 116) | | | | | | | | | |
| CGTTGCCAAAGAGTGACCTGAGG | AF033819.3: 1799 | − | 55 | 0 | 0 | 0 | 0 | 0 | 68.43 |
| (SEQ ID NO: 117) | | | | | | | | | |
| TCGTCACAATAAAGATAGGGGGG | AF033819.3: 1827 | + | 40 | 0 | 0 | 0 | 0 | 0 | 68.43 |
| (SEQ ID NO: 118) | | | | | | | | | |
| GAAGGCTGTAGACAAATACTGGG | AF033819.3: 498 | + | 40 | 0 | 0 | 0 | 0 | 0 | 67.76 |
| (SEQ ID NO: 119) | | | | | | | | | |
| ACCATGCTAAACACAGTGGGGGG | AF033819.3: 891 | + | 50 | 0 | 0 | 0 | 0 | 0 | 67.73 |
| (SEQ ID NO: 120) | | | | | | | | | |
| GGCCTGTTAGAAACATCAGAAGG | AF033819.3: 480 | + | 45 | 1 | 0 | 0 | 0 | 0 | 68.53 |
| (SEQ ID NO: 121) | | | | | | | | | |
| AGCCGAGCAAGCTTCACAGGAGG | AF033819.3: 1250 | + | 60 | 1 | 0 | 0 | 0 | 0 | 68.45 |
| (SEQ ID NO: 122) | | | | | | | | | |
| CAGGCCAGATGAGAGAACCAAGG | AF033819.3: 1009 | + | 55 | 0 | 0 | 0 | 0 | 0 | 67.18 |
| (SEQ ID NO: 123) | | | | | | | | | |
| GTTGCCAAAGAGTGACCTGAGGG | AF033819.3: 1798 | − | 50 | 0 | 0 | 0 | 0 | 0 | 66.99 |
| (SEQ ID NO: 124) | | | | | | | | | |
| GGACATAAGACAAGGACCAAAGG | AF033819.3: 1184 | + | 45 | 2 | 0 | 0 | 0 | 0 | 68.5 |
| (SEQ ID NO: 125) | | | | | | | | | |
| TATCGGCTCCTGCTTCTGAGGGG | AF033819.3: 1750 | − | 55 | 1 | 0 | 0 | 0 | 0 | 66.67 |
| (SEQ ID NO: 126) | | | | | | | | | |
| GGGCTGTTGGAAATGTGGAAAGG | AF033819.3: 1568 | + | 50 | 0 | 0 | 0 | 0 | 0 | 64.65 |
| (SEQ ID NO: 127) | | | | | | | | | |

gag (AAC82593.1)|Cpf1 CasX

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| TTCTAGTGTAGCCGCTGGTCCCAATGCT | AF033819.3: 1340 | − | 58 | 0 | 0 | 0 | 0 | 0 | — |
| (SEQ ID NO: 128) | | | | | | | | | |
| TTCTTCTAGTGTAGCCGCTGGTCCCAAT | AF033819.3: 1343 | − | 54 | 0 | 0 | 0 | 0 | 0 | — |
| (SEQ ID NO: 129) | | | | | | | | | |
| TTCAGCCAAAACTCTTGCCTTATGGCCG | AF033819.3: 1403 | − | 54 | 0 | 0 | 0 | 0 | 0 | — |
| (SEQ ID NO: 130) | | | | | | | | | |
| TTCAGCTACCATAATGATGCAGAGAGGC | AF033819.3: 1451 | + | 50 | 0 | 0 | 0 | 0 | 0 | — |
| (SEQ ID NO: 131) | | | | | | | | | |
| TTCCTAAAATTGCCTCTCTGCATCATTA | AF033819.3: 1462 | − | 33 | 0 | 0 | 0 | 0 | 0 | — |
| (SEQ ID NO: 132) | | | | | | | | | |
| TTCTTTGGTTCCTAAAATTGCCTCTCTG | AF033819.3: 1470 | − | 42 | 0 | 0 | 0 | 0 | 0 | — |
| (SEQ ID NO: 133) | | | | | | | | | |
| TTCTTTGCCACAATTGAAACACTTAACA | AF033819.3: 1502 | − | 33 | 0 | 0 | 0 | 0 | 0 | — |
| (SEQ ID NO: 134) | | | | | | | | | |
| TTCTGGCTGTGTGCCCTTCTTTGCCACA | AF033819.3: 1518 | − | 58 | 0 | 0 | 0 | 0 | 0 | — |
| (SEQ ID NO: 135) | | | | | | | | | |
| TTCCACATTTCCAACAGCCCTTTTTCCT | AF033819.3: 1560 | − | 42 | 0 | 0 | 0 | 0 | 0 | — |
| (SEQ ID NO: 136) | | | | | | | | | |
| TTCCTTTCCACATTTCCAACAGCCCTTT | AF033819.3: 1565 | − | 42 | 0 | 0 | 0 | 0 | 0 | — |
| (SEQ ID NO: 137) | | | | | | | | | |
| TTCTAGCTCCCTGCTTGCCCATACTATA | AF033819.3: 434 | − | 50 | 0 | 0 | 0 | 0 | 0 | — |
| (SEQ ID NO: 138) | | | | | | | | | |
| TTCATTTGGTGTCCTTCCTTTCCACATT | AF033819.3: 1579 | − | 42 | 0 | 0 | 0 | 0 | 0 | — |
| (SEQ ID NO: 139) | | | | | | | | | |
| TTCCCTAAAAAATTAGCCTGTCTCTCAG | AF033819.3: 1615 | − | 38 | 0 | 0 | 0 | 0 | 0 | — |
| (SEQ ID NO: 140) | | | | | | | | | |
| TTCCTACAAGGGAAGGCCAGGGAATTTT | AF033819.3: 1652 | + | 46 | 0 | 0 | 0 | 0 | 0 | — |
| (SEQ ID NO: 141) | | | | | | | | | |
| TTCTTCAGAGCAGACCAGAGCCAACAGC | AF033819.3: 1678 | + | 58 | 0 | 0 | 0 | 0 | 0 | — |
| (SEQ ID NO: 142) | | | | | | | | | |
| TTCAGAGCAGACCAGAGCCAACAGCCCC | AF033819.3: 1681 | + | 67 | 0 | 0 | 0 | 0 | 0 | — |
| (SEQ ID NO: 143) | | | | | | | | | |
| TTCTGGTGGGGCTGTTGGCTCTGGTCTG | AF033819.3: 1688 | − | 67 | 0 | 0 | 0 | 0 | 0 | — |
| (SEQ ID NO: 144) | | | | | | | | | |
| TTCTGAGGGGGAGTTGTTGTCTCTACCC | AF033819.3: 1732 | − | 58 | 0 | 0 | 0 | 0 | 0 | — |
| (SEQ ID NO: 145) | | | | | | | | | |
| TTCCTTGTCTATCGGCTCCTGCTTCTGA | AF033819.3: 1754 | − | 50 | 0 | 0 | 0 | 0 | 0 | — |
| (SEQ ID NO: 146) | | | | | | | | | |
| TTCCCTCAGGTCACTCTTTGGCAACGAC | AF033819.3: 1796 | + | 54 | 0 | 0 | 0 | 0 | 0 | — |
| (SEQ ID NO: 147) | | | | | | | | | |

tat (AAC82591.1)|Cas9

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| GAAGGAATAGAAGAAGAAGGTGG | AF033819.3: 7961 | + | 40 | 0 | 0 | 0 | 0 | 0 | 68.86 |
| (SEQ ID NO: 148) | | | | | | | | | |
| ACCCACCTCCCAACCCCGAGGGG | AF033819.3: 7925 | + | 70 | 0 | 0 | 0 | 0 | 0 | 65.55 |
| (SEQ ID NO: 149) | | | | | | | | | |
| AGACCCACCTCCCAACCCCGAGG | AF033819.3: 7923 | + | 70 | 0 | 0 | 0 | 0 | 0 | 64.06 |
| (SEQ ID NO: 150) | | | | | | | | | |
| GGGCCTGTCGGGTCCCCTCGGGG | AF033819.3: 7938 | − | 80 | 2 | 0 | 0 | 0 | 0 | 61.03 |
| (SEQ ID NO: 151) | | | | | | | | | |
| GACCCACCTCCCAACCCCGAGGG | AF033819.3: 7924 | + | 70 | 0 | 0 | 0 | 0 | 0 | 56.4 |
| (SEQ ID NO: 152) | | | | | | | | | |
| AGGGGACCCGACAGGCCCGAAGG | AF033819.3: 7943 | + | 75 | 1 | 0 | 0 | 0 | 0 | 56.72 |
| (SEQ ID NO: 153) | | | | | | | | | |
| CCCGAAGGAATAGAAGAAGAAGG | AF033819.3: 7958 | + | 45 | 0 | 0 | 0 | 0 | 0 | 55.43 |
| (SEQ ID NO: 154) | | | | | | | | | |
| AGGTGGGTCTGAAACGATAATGG | AF033819.3: 7910 | − | 45 | 0 | 0 | 0 | 0 | 0 | 49.71 |
| (SEQ ID NO: 155) | | | | | | | | | |
| CAACCCCGAGGGGACCCGACAGG | AF033819.3: 7935 | + | 75 | 2 | 0 | 0 | 0 | 0 | 49.14 |
| (SEQ ID NO: 156) | | | | | | | | | |
| TCCCCTCGGGGTTGGGAGGTGGG | AF033819.3: 7926 | − | 70 | 1 | 0 | 0 | 0 | 0 | 41.54 |
| (SEQ ID NO: 157) | | | | | | | | | |
| CGGGTCCCCTCGGGGTTGGGAGG | AF033819.3: 7930 | − | 80 | 3 | 0 | 0 | 0 | 0 | 42.94 |
| (SEQ ID NO: 158) | | | | | | | | | |
| TTCTATTCCTTCGGGCCTGTCGG | AF033819.3: 7950 | − | 50 | 0 | 0 | 0 | 0 | 0 | 38.96 |
| (SEQ ID NO: 159) | | | | | | | | | |
| TCTATTCCTTCGGGCCTGTCGGG | AF033819.3: 7949 | − | 55 | 0 | 0 | 0 | 0 | 0 | 37.89 |
| (SEQ ID NO: 160) | | | | | | | | | |
| CGGGCCTGTCGGGTCCCCTCGGG | AF033819.3: 7939 | − | 80 | 1 | 0 | 0 | 0 | 0 | 36.86 |
| (SEQ ID NO: 161) | | | | | | | | | |
| TCGGGCCTGTCGGGTCCCCTCGG | AF033819.3: 7940 | − | 75 | 1 | 0 | 0 | 0 | 0 | 36.35 |
| (SEQ ID NO: 162) | | | | | | | | | |
| ACCTTCTTCTTCTATTCCTTCGG | AF033819.3: 7959 | − | 35 | 0 | 0 | 0 | 0 | 0 | 32.23 |
| (SEQ ID NO: 163) | | | | | | | | | |
| TGTCGGGTCCCCTCGGGGTTGGG | AF033819.3: 7933 | − | 70 | 2 | 0 | 0 | 0 | 0 | 32.59 |
| (SEQ ID NO: 164) | | | | | | | | | |
| CTGTCGGGTCCCCTCGGGGTTGG | AF033819.3: 7934 | − | 75 | 2 | 0 | 0 | 0 | 0 | 30.87 |
| (SEQ ID NO: 165) | | | | | | | | | |
| GTCCCCTCGGGGTTGGGAGGTGG | AF033819.3: 7927 | − | 75 | 1 | 0 | 0 | 0 | 0 | 25.53 |
| (SEQ ID NO: 166) | | | | | | | | | |

tat (AAC82591.1)|Cpf1 CasX

| | | | GC | Self- | | | | | |
|---|---|---|---|---|---|---|---|---|---|
| | Genomic | | Content | complemen- | | | | | Effi- |
| Target Sequence | Location | Strand | (%) | tarity | MM0 | MM1 | MM2 | MM3 | ciency |
| TTCGGGCCTGTCGGGTCCCCTCGGGGTT | AF033819.3: 7936 | − | 75 | 0 | 0 | 0 | 0 | 0 | — |
| (SEQ ID NO: 167) | | | | | | | | | |
| TTCCTTCGGGCCTGTCGGGTCCCCTCGG | AF033819.3: 7940 | − | 75 | 0 | 0 | 0 | 0 | 0 | — |
| (SEQ ID NO: 168) | | | | | | | | | |
| TTCTATTCCTTCGGGCCTGTCGGGTCCC | AF033819.3: 7945 | − | 67 | 0 | 0 | 0 | 0 | 0 | — |
| (SEQ ID NO: 169) | | | | | | | | | |
| TTCTTCTATTCCTTCGGGCCTGTCGGGT | AF033819.3: 7948 | − | 58 | 0 | 0 | 0 | 0 | 0 | — |
| (SEQ ID NO: 170) | | | | | | | | | |
| TTCTTCTTCTATTCCTTCGGGCCTGTCG | AF033819.3: 7951 | − | 54 | 0 | 0 | 0 | 0 | 0 | — |
| (SEQ ID NO: 171) | | | | | | | | | |
| TTCACCATTATCGTTTCAGACCCACCTC | AF033819.3: 7906 | + | 50 | 0 | 0 | 0 | 0 | 0 | — |
| (SEQ ID NO: 172) | | | | | | | | | |
| TTCAGACCCACCTCCCAACCCCGAGGGG | AF033819.3: 7920 | + | 75 | 2 | 0 | 0 | 0 | 0 | — |
| (SEQ ID NO: 173) | | | | | | | | | |

5′LTR Sequence of HXB2|Cas9

| | | | GC | Self- | | | | | |
|---|---|---|---|---|---|---|---|---|---|
| | Sequence | | content | complemen- | | | | | Effi- |
| Target sequence | location | Strand | (%) | tarity | MM0 | MM1 | MM2 | MM3 | ciency |
| ATTAGCAGAACTACACACCAGGG | seq: 79 | + | 40 | 1 | 1 | 0 | 0 | 0 | 74.78 |
| (SEQ ID NO: 174) | | | | | | | | | |
| TAGCTTGTAGCACCATCCAAAGG | seq: 124 | − | 45 | 0 | 1 | 0 | 0 | 0 | 66.77 |
| (SEQ ID NO: 175) | | | | | | | | | |
| AGAACTACACACCAGGGCCAGGG | seq: 85 | + | 55 | 0 | 1 | 0 | 0 | 0 | 66.46 |
| (SEQ ID NO: 176) | | | | | | | | | |
| CAGGGAAGTAGCCTTGTGTGTGG | seq: 56 | − | 55 | 0 | 1 | 0 | 0 | 0 | 66.2 |
| (SEQ ID NO: 177) | | | | | | | | | |
| TCAGACCCTTTTAGTCAGTGTGG | seq: 599 | + | 45 | 0 | 1 | 0 | 0 | 0 | 63.95 |
| (SEQ ID NO: 178) | | | | | | | | | |
| CTGTGGATCTACCACACACAAGG | seq: 45 | + | 50 | 2 | 1 | 0 | 0 | 0 | 65.44 |
| (SEQ ID NO: 179) | | | | | | | | | |
| ACCAGAGTCACACAACAGACGGG | seq: 563 | − | 50 | 1 | 1 | 0 | 0 | 0 | 64.21 |
| (SEQ ID NO: 180) | | | | | | | | | |
| GATATCCACTGACCTTTGGATGG | seq: 112 | + | 45 | 1 | 1 | 0 | 0 | 0 | 64.02 |
| (SEQ ID NO: 181) | | | | | | | | | |
| GACAAGATATCCTTGATCTGTGG | seq: 28 | + | 40 | 1 | 1 | 0 | 0 | 0 | 63.82 |
| (SEQ ID NO: 182) | | | | | | | | | |
| ACACTGACTAAAAGGGTCTGAGG | seq: 597 | − | 45 | 0 | 1 | 0 | 0 | 0 | 62.31 |
| (SEQ ID NO: 183) | | | | | | | | | |
| TAACCAGAGAGACCCAGTACAGG | seq: 446 | − | 50 | 0 | 1 | 0 | 0 | 0 | 62.05 |
| (SEQ ID NO: 184) | | | | | | | | | |
| TAGCACCATCCAAAGGTCAGTGG | seq: 117 | − | 50 | 0 | 1 | 0 | 0 | 0 | 61.47 |
| (SEQ ID NO: 185) | | | | | | | | | |
| TACCAGAGTCACACAACAGACGG | seq: 564 | − | 45 | 1 | 1 | 0 | 0 | 0 | 60.14 |
| (SEQ ID NO: 186) | | | | | | | | | |
| GATTAGCAGAACTACACACCAGG | seq: 78 | + | 45 | 1 | 1 | 0 | 0 | 0 | 59.79 |
| (SEQ ID NO: 187) | | | | | | | | | |
| AGAGAGAAGTGTTAGAGTGGAGG | seq: 241 | + | 45 | 0 | 1 | 0 | 0 | 0 | 58.16 |
| (SEQ ID NO: 188) | | | | | | | | | |
| CACTGACTAAAAGGGTCTGAGGG | seq: 596 | − | 45 | 0 | 1 | 0 | 0 | 0 | 58.08 |
| (SEQ ID NO: 189) | | | | | | | | | |
| CTACAAGGGACTTTCCGCTGGGG | seq: 344 | + | 55 | 2 | 1 | 0 | 0 | 0 | 59.28 |
| (SEQ ID NO: 190) | | | | | | | | | |
| GCTCACAGGGTGTAACAAGCTGG | seq: 196 | − | 55 | 0 | 1 | 0 | 0 | 0 | 56.6 |
| (SEQ ID NO: 191) | | | | | | | | | |
| CACACTACTTGAAGCACTCAAGG | seq: 538 | − | 45 | 1 | 1 | 0 | 0 | 0 | 57.53 |
| (SEQ ID NO: 192) | | | | | | | | | |
| ACTCTAACACTTCTCTCTCCGGG | seq: 236 | − | 45 | 1 | 1 | 0 | 0 | 0 | 56.98 |
| (SEQ ID NO: 193) | | | | | | | | | |

5′LTR Sequence of HXB2|Cpf1 CasX

| | | | GC | Self- | | | | | |
|---|---|---|---|---|---|---|---|---|---|
| | Sequence | | content | complemen- | | | | | Effi- |
| Target sequence | location | Strand | (%) | tarity | MM0 | MM1 | MM2 | MM3 | ciency |
| TTCTCTGGCTCAACTGGTACTAGCTTGT | seq: 139 | − | 50 | 0 | 0 | 0 | 0 | 0 | — |
| (SEQ ID NO: 194) | | | | | | | | | |
| TTCTTCTAACTTCTCTGGCTCAACTGGT | seq: 149 | − | 46 | 0 | 0 | 0 | 0 | 0 | — |
| (SEQ ID NO: 195) | | | | | | | | | |
| TTCCATGCAGGCTCACAGGGTGTAACAA | seq: 201 | − | 50 | 0 | 0 | 0 | 0 | 0 | — |
| (SEQ ID NO: 196) | | | | | | | | | |
| TTCTTGAAGTACTCCGGATGCAGCTCTC | seq: 298 | − | 54 | 0 | 0 | 0 | 0 | 0 | — |
| (SEQ ID NO: 197) | | | | | | | | | |
| TTCAAGAACTGCTGACATCGAGCTTGCT | seq: 318 | + | 50 | 0 | 0 | 0 | 0 | 0 | — |
| (SEQ ID NO: 198) | | | | | | | | | |
| TTCAAGTAGTGTGTGCCCGTCTGTTGTG | seq: 548 | + | 54 | 0 | 0 | 0 | 0 | 0 | — |
| (SEQ ID NO: 199) | | | | | | | | | |
| TTCCACACTGACTAAAAGGGTCTGAGGG | seq: 596 | − | 50 | 0 | 0 | 0 | 0 | 0 | — |
| (SEQ ID NO: 200) | | | | | | | | | |
| TTCCCTGATTAGCAGAACTACACACCAG | seq: 72 | + | 46 | 0 | 0 | 0 | 0 | 0 | — |
| (SEQ ID NO: 201) | | | | | | | | | |
| TTCCGCTGGGGACTTTCCAGGGAGGCGT | seq: 356 | + | 67 | 1 | 0 | 0 | 0 | 0 | — |
| (SEQ ID NO: 202) | | | | | | | | | |
| TTCCAGGGAGGCGTGGCCTGGGGGGGAC | seq: 370 | + | 79 | 1 | 0 | 0 | 0 | 0 | — |
| (SEQ ID NO: 203) | | | | | | | | | |
| TTCTGCTAATCAGGGAAGTAGCCTTGTG | seq: 61 | − | 50 | 1 | 0 | 0 | 0 | 0 | — |
| (SEQ ID NO: 204) | | | | | | | | | |
| TTCTCTCCTTTGTTGGCTTCTTCTAACT | seq: 166 | − | 42 | 0 | 0 | 0 | O | 0 | — |
| (SEQ ID NO: 205) | | | | | | | | | |
| TTCTAACTTCTCTGGCTCAACTGGTACT | seq: 146 | − | 46 | 0 | 0 | 0 | 0 | 0 | — |
| (SEQ ID NO: 206) | | | | | | | | | |
| TTCACTCCCAACGAAGACAAGATATCCT | seq: 13 | + | 46 | 0 | 0 | 0 | 0 | 0 | — |
| (SEQ ID NO: 207) | | | | | | | | | |
| TTCTCTCTCCGGGTCATCCATTCCATGC | seq: 221 | − | 58 | 0 | 0 | 0 | 0 | 0 | — |
| (SEQ ID NO: 208) | | | | | | | | | |
| TTCATCACATGGCCCGAGAGCTGCATCC | seq: 283 | + | 62 | 0 | 0 | 0 | 0 | 0 | — |
| (SEQ ID NO: 209) | | | | | | | | | |
| TTCCCTAGTTAGCCAGAGAGCTCCCAGG | seq: 482 | − | 58 | 0 | 0 | 0 | 0 | 0 | — |
| (SEQ ID NO: 210) | | | | | | | | | |

3′LTR Sequence of HXB2|Cas9

| | | | GC | Self- | | | | | |
|---|---|---|---|---|---|---|---|---|---|
| | Sequence | | content | complemen- | | | | | Effi- |
| Target sequence | location | Strand | (%) | tarity | MM0 | MM1 | MM2 | MM3 | ciency |
| ATTAGCAGAACTACACACCAGGG | seq: 164 | + | 40 | 1 | 1 | 0 | 0 | 0 | 74.78 |
| (SEQ ID NO: 211) | | | | | | | | | |
| ATTGGTCTTAAAGGTACCTGAGG | seq: 10 | − | 40 | 0 | 1 | 0 | 0 | 0 | 68.73 |
| (SEQ ID NO: 212) | | | | | | | | | |
| TAGCTTGTAGCACCATCCAAAGG | seq: 209 | − | 45 | 0 | 1 | 0 | 0 | 0 | 66.77 |
| (SEQ ID NO: 213) | | | | | | | | | |
| GAACTACACACCAGGGCCAGGGG | seq: 171 | + | 60 | 0 | 1 | 0 | 0 | 0 | 66.69 |
| (SEQ ID NO: 214) | | | | | | | | | |
| CAGGGAAGTAGCCTTGTGTGTGG | seq: 141 | − | 55 | 0 | 1 | 0 | 0 | 0 | 66.2 |
| (SEQ ID NO: 215) | | | | | | | | | |
| TCAGACCCTTTTAGTCAGTGTGG | seq: 684 | + | 45 | 0 | 1 | 0 | 0 | 0 | 63.95 |
| (SEQ ID NO: 216) | | | | | | | | | |
| CTGTGGATCTACCACACACAAGG | seq: 130 | + | 50 | 2 | 1 | 0 | 0 | 0 | 65.44 |
| (SEQ ID NO: 217) | | | | | | | | | |
| ACCAGAGTCACACAACAGACGGG | seq: 648 | − | 50 | 1 | 1 | 0 | 0 | 0 | 64.21 |
| (SEQ ID NO: 218) | | | | | | | | | |
| GATATCCACTGACCTTTGGATGG | seq: 197 | + | 45 | 1 | 1 | 0 | 0 | 0 | 64.02 |
| (SEQ ID NO: 219) | | | | | | | | | |
| GACAAGATATCCTTGATCTGTGG | seq: 113 | + | 40 | 1 | 1 | 0 | 0 | 0 | 63.82 |
| (SEQ ID NO: 220) | | | | | | | | | |
| AGAACTACACACCAGGGCCAGGG | seq: 170 | + | 55 | 0 | 1 | 0 | 0 | 0 | 62.62 |
| (SEQ ID NO: 221) | | | | | | | | | |
| ACACTGACTAAAAGGGTCTGAGG | seq: 682 | − | 45 | 0 | 1 | 0 | 0 | 0 | 62.31 |
| (SEQ ID NO: 222) | | | | | | | | | |
| GGGCCACGTGATGAAATGCTAGG | seq: 360 | − | 55 | 2 | 1 | 0 | 0 | 0 | 64.29 |
| (SEQ ID NO: 223) | | | | | | | | | |
| TAACCAGAGAGACCCAGTACAGG | seq: 531 | − | 50 | 0 | 1 | 0 | 0 | 0 | 62.05 |
| (SEQ ID NO: 224) | | | | | | | | | |
| TAGCACCATCCAAAGGTCAGTGG | seq: 202 | − | 50 | 0 | 1 | 0 | 0 | 0 | 61.47 |
| (SEQ ID NO: 225) | | | | | | | | | |
| CACTTTTTAAAAGAAAAGGGGGG | seq: 61 | + | 30 | 0 | 1 | 0 | 0 | 0 | 61.16 |
| (SEQ ID NO: 226) | | | | | | | | | |
| TGAGCCAGATAAGATAGAAGAGG | seq: 240 | + | 40 | 1 | 1 | 0 | 0 | 0 | 61.05 |
| (SEQ ID NO: 227) | | | | | | | | | |
| TACCAGAGTCACACAACAGACGG | seq: 649 | − | 45 | 1 | 1 | 0 | 0 | 0 | 60.14 |
| (SEQ ID NO: 228) | | | | | | | | | |
| GATTAGCAGAACTACACACCAGG | seq: 163 | + | 45 | 1 | 1 | 0 | 0 | 0 | 59.79 |
| (SEQ ID NO: 229) | | | | | | | | | |
| AGAGAGAAGTGTTAGAGTGGAGG | seq: 326 | + | 45 | 0 | 1 | 0 | 0 | 0 | 58.16 |
| (SEQ ID NO: 230) | | | | | | | | | |

3′LTR Sequence of HXB2|Cpf1 CasX

| | | | GC | Self- | | | | | |
|---|---|---|---|---|---|---|---|---|---|
| | Sequence | | content | complemen- | | | | | Effi- |
| Target sequence | location | Strand | (%) | tarity | MM0 | MM1 | MM2 | MM3 | ciency |
| TTCCCTGATTAGCAGAACTACACACCAG | seq: 157 | + | 46 | 0 | 1 | 0 | 0 | 0 | — |
| (SEQ ID NO: 231) | | | | | | | | | |
| TTCTATCTTATCTGGCTCAACTGGTACT | seq: 231 | − | 42 | 0 | 1 | 0 | 0 | 0 | — |
| (SEQ ID NO: 232) | | | | | | | | | |
| TTCTCTCCTTTATTGGCCTCTTCTATCT | seq: 251 | − | 42 | 0 | 1 | 0 | 0 | 0 | — |
| (SEQ ID NO: 233) | | | | | | | | | |
| TTCTCTCTCCGGGTCATCCATCCCATGC | seq: 306 | − | 62 | 0 | 1 | 0 | 0 | 0 | — |
| (SEQ ID NO: 234) | | | | | | | | | |
| TTCATCACGTGGCCCGAGAGCTGCATCC | seq: 368 | + | 67 | 0 | 1 | 0 | 0 | 0 | — |
| (SEQ ID NO: 235) | | | | | | | | | |
| TTCTTGAAGTACTCCGGATGCAGCTCTC | seq: 383 | − | 54 | 0 | 1 | 0 | 0 | 0 | — |
| (SEQ ID NO: 236) | | | | | | | | | |
| TTCAAGAACTGCTGACATCGAGCTTGCT | seq: 403 | + | 50 | 0 | 1 | 0 | 0 | 0 | — |
| (SEQ ID NO: 237) | | | | | | | | | |
| TTCTTTTAAAAAGTGGCTAAGATCTACA | seq: 48 | − | 29 | 0 | 1 | 0 | 0 | 0 | — |
| (SEQ ID NO: 238) | | | | | | | | | |
| TTCCAGTCCCCCCTTTTCTTTTAAAAAG | seq: 63 | | 38 | 0 | 1 | 0 | C | 0 | — |
| (SEQ ID NO: 239) | | | | | | | | | |
| TTCAAGTAGTGTGTGCCCGTCTGTTGTG | seq: 633 | + | 54 | 0 | 1 | 0 | 0 | 0 | — |
| (SEQ ID NO: 240) | | | | | | | | | |
| TTCCACACTGACTAAAAGGGTCTGAGGG | seq: 681 | − | 50 | 0 | 1 | 0 | 0 | 0 | — |
| (SEQ ID NO: 241) | | | | | | | | | |
| TTCTTTGGGAGTGAATTAGCCCTTCCAG | seq: 85 | − | 50 | 0 | 1 | 0 | 0 | 0 | — |
| (SEQ ID NO: 242) | | | | | | | | | |
| TTCACTCCCAAAGAAGACAAGATATCCT | seq: 98 | + | 42 | 0 | 1 | 0 | 0 | 0 | — |
| (SEQ ID NO: 243) | | | | | | | | | |
| TTCTGCTAATCAGGGAAGTAGCCTTGTG | seq: 146 | − | 50 | 1 | 1 | 0 | 0 | 0 | — |
| (SEQ ID NO: 244) | | | | | | | | | |
| TTCCGCTGGGGACTTTCCAGGGAGGCGT | seq: 441 | + | 67 | 1 | 1 | 0 | 0 | 0 | — |
| (SEQ ID NO: 245) | | | | | | | | | |
| TTCCAGGGAGGCGTGGCCTGGGCGGGAC | seq: 455 | + | 79 | 1 | 1 | 0 | 0 | 0 | |
| (SEQ ID NO: 246) | | | | | | | | | |
| TTCCCTAGTTAGCCAGAGAGCTCCCAGG | seq: 567 | − | 58 | 0 | 2 | 0 | 0 | 0 | — |
| (SEQ ID NO: 247) | | | | | | | | | |

HXB2 TAR gggtctctggttagaccagatctgagcctgggagctct
ctggctaactagggaaccca (SEQ ID NO: 248)|Cas9

| | | | GC | Self- | | | | | |
|---|---|---|---|---|---|---|---|---|---|
| | Sequence | | content | complemen- | | | | | Effi- |
| Target sequence | location | Strand | (%) | tarity | MM0 | MM1 | MM2 | MM3 | ciency |
| GTTAGACCAGATCTGAGCCTGGG | seq: 12 | + | 50 | 1 | 2 | 0 | 0 | 0 | 62.78 |
| (SEQ ID NO: 249) | | | | | | | | | |
| TAGTTAGCCAGAGAGCTCCCAGG | seq: 29 | − | 55 | 0 | 2 | 0 | 0 | 0 | 57.48 |
| (SEQ ID NO: 250) | | | | | | | | | |
| GGGAGCTCTCTGGCTAACTAGGG | seq: 32 | + | 55 | 0 | 2 | 0 | 0 | 0 | 55.28 |
| (SEQ ID NO: 251) | | | | | | | | | |
| GGTTAGACCAGATCTGAGCCTGG | seq: 11 | + | 55 | 0 | 2 | 0 | 0 | 0 | 52.01 |
| (SEQ ID NO: 252) | | | | | | | | | |
| AGAGCTCCCAGGCTCAGATCTGG | seq: 18 | − | 60 | 3 | 2 | 0 | 0 | 0 | 46.76 |
| (SEQ ID NO: 253) | | | | | | | | | |
| ATCTGAGCCTGGGAGCTCTCTGG | seq: 22 | + | 60 | 3 | 2 | 0 | 0 | 0 | 45.08 |
| (SEQ ID NO: 253) | | | | | | | | | |
| TGGGAGCTCTCTGGCTAACTAGG | seq: 31 | + | 55 | 0 | 2 | 0 | 0 | 0 | 32.08 |
| (SEQ ID NO: 254) | | | | | | | | | |

HXB2 TAR gggtctctggttagaccagatctgagcctgggagctctctg
gctaactagggaaccca (SEQ ID NO: 248)|Cpf1 CasX

| | | | GC | Self- | | | | | |
|---|---|---|---|---|---|---|---|---|---|
| | Sequence | | content | complemen- | | | | | Effi- |
| Target sequence | location | Strand | (%) | tarity | MM0 | MM1 | MM2 | MM3 | ciency |
| TTCCCTAGTTAGCCAGAGAGCTCCCAGG | seq: 29 | − | 58 | 0 | 2 | 0 | 0 | — | 62.78 |
| (SEQ ID NO: 255) | | | | | | | | | |

The cell lines, animals, and gRNAs described herein can be used to evaluate the differences in efficiency of BioNV payload delivery versus AAV delivery of the same payload into a desired cell line. The cell line(s) can be any CD4+ cell line. Alternate cell lines can be used to evaluate the effect in other latent viruses. The degree of gRNA binding to the proviral DNA can be evaluated using tools such as pinpoint FISH analysis. The degree/efficiency of excision by the gRNAs and the Cas endonuclease can be monitored using PCR and/or sequencing to detect both dropout sequences and indel junctions relative to adjacent PAMs, in parallel with reporter system readouts (loss or gain of function—depending on the reporter system). gRNAs can also be evaluated in vitro in each cell line using reporter assays, etc. to measure effective excision between gRNA-endonuclease-HIV sequence pairings.

Nef-related strategies can include complete or partial knockout of Nef by CRISPR/Cas or the silencing of full length or partial Nef by shRNA in combination with CRISPR/Cas excision of the proviral genome 5′ to the Nef gene. This can be evaluated by immunoblotting cell extracts from cells treated with gRNAs that knockout Nef entirely, looking for positive versus negative Nef signal in treated cells using α-Nef antibodies; immunoblotting cellular extracts from cells treated with gRNAs that knockout partial Nef, using α-partial Nef antibodies; and/or immunoblotting cellular extracts from cells transfected (and/or transduced) with gRNAs and Cas9 (or other endonucleases) that excise outside of Nef (e.g., Nef remains intact for shRNA targeting), and shRNAs that silence Nef. Time course qPCR experiments can be done in parallel with northern blotting to observe the effect on the Nef mRNA levels. A parallel HIV reporter assay readout and/or PCR can be used to detect the level of excision that does not excise Nef.

The degree of shRNA spread among cells can be evaluated by serially transducing HIV reporter cell lines that express diminishing amounts of shRNA targeting Nef with CRISPR Cas/gRNA loaded BioNVs (and AAVs). The experiment can be designed to show a ‘spreading’ effect among cells in culture from high levels to low levels of gene editing system. An HIV reporter cell line with a knockout of SIDT1/2 can be used to compare the results with HIV reporter WT SIDT1/2 cell lines. This can also be measured by observing the change of concentration of MHC on cell surfaces of adjacent cells. Control experiments can be performed to observe any interference that can occur from the Nef mRNA pool that has been predicted to be stable, and the effectiveness of the approach to silence that pool. Flow cytometry and/or immunoblotting in parallel can be used to track the replenishment of MHC in cell populations as the signal shRNA Nef silencing spreads in the culture. These experiments can evaluate the degree of shRNA Nef silencing and cell to cell spreading related to the concentration of CRISPR Cas9/gRNA/shRNA plasmid (or mRNA) delivered in culture. This can be used to establish a baseline for future in vivo dosing.

The degree of off targeting effects caused by the CRISPR/Cas gRNA system can be evaluated by ultra-deep next generation global genomic sequencing. PCR and/or deep sequencing can be used to measure the degree of excision in neural stem cells by observing dropout sequences and indel junctions relative to adjacent PAMs. PCR can be used to measure the degree of presence/absence of Nef transcripts in parallel with immunoblotting to measure the degree of presence/absence of Nef protein. PCR can be used to measure the degree of presence/absence of Nef transcripts in parallel with immunoblotting to measure the degree of presence/absence of Nef protein in cells and which can be expanded to tissue sections, where the RNA scope can be observed to measure the degree of spread of the signal throughout the tissue.

The animal models can provide insight into the degree of excision and the spread of shRNA counter-Nef signaling in vivo. The events can be measured using one or more of immunoblotting, PCR, reporter system tracking, and RNA scope for tissue samples.

To study the potential immune response from CD8+ T cells after Nef silencing and release of MHC complexes to the surface of the cells harboring both active and latent proviral peptides (post excision), the functional CD8+ T cell populations can be isolated from BLT mice (and/or CD34+ NSG mice) that have been treated and/or primed with the CRISPR/Cas gRNA+shRNA anti-Nef constructs and compare them to CD8+ T cell populations from untreated mice. In vitro and/or in vivo “rescue” adoptive transfer-type assays can be used to observe CD8+ T cell killing of infected cells. For example, in vitro assays can include the addition of primed CD8+ T cells to infected CD4+ T cell populations in culture over a concentration and time course-dependent manner to establish infected CD4+ kill curves which can be measured by cytometry (e.g., counting the degree of cell death), qPCR of apoptotic induced mRNA signals such as BAD, BAX and caspase-3, and chromium-51 (⁵¹Cr) release cytotoxicity assays. The increase in apoptotic cytokines in the primed CD8+ T-cell population (pre- and post-introduction to the infected CD4+ population) such as perforin and granzymes, can also be measured directly from cell lysates (mRNA/protein measurements) and/or media lysates (secreted cytokine concentrations).

In vivo studies can be used to reintroduce the primed CD8+ T cells into mice and challenge via HIV infection in BLT mice, with or without ART co-therapy. This can establish if the CD8+ T cell population can protect the mice from new infection. Primed CD8+ T cells can be introduced into Rag2J-γc−/− mice, pre-infection (infecting the animals with HIV after treatment), followed by observation of the effects on viral loads and survivability. HIV-infected BLT mice (and/or Rag2−/− γc−/− mice) can be treated by reintroducing the primed CD8+ T cells into the mice post-infection, in an increasing concentration over a time course, observing the effect the CD8+ T cells have on the CD4+ cell population that has an established pool of provirus. Standard CD4+/primed CD8+ T cell counting methods can be used to measure over a time course and measure viral load using standard/established protocols. These experiments can be repeated in mouse models inoculated with cell free EcoHIV-eLuc reporter virus to measure the degree of viral elimination/deactivation using a visualization marker.

To measure the biodistribution of the BioNV against AAV delivery of CRISPR Cas/gRNAs and shRNA anti-Nef, an analysis of the tissues of the BLT mouse models can be performed using combinations of PCR, RNA scope, and/or sequencing. A visualization marker, such as GFP, or luciferase reporter constructs, can be used in parallel with the deliverable(s) to visually track its biodistribution properties within the tissues of the animals.

The evaluate the degree of RNA interference off-targeting in human transcripts, deep sequencing and hybridization chip array analysis of messenger RNAs in CD4+ cell populations can be used, as well as in the tissues of animal models. To assess the degree of viral rebound after each treatment strategy, CD34+ mouse models can be used with established and published protocols for evaluating viral rebound.

The biodistribution, excision efficiency, shRNA silencing efficiency of Nef, RNAi signal spreading among cells and tissues, and CD8+ priming assays mentioned herein can be repeated in non-human primate models. This can be done to confirm the findings in the mouse models and to further evaluate the safety and efficacy of the CRISPR-Cas9 system in these models. Viral rebound post-ART therapy and post treatment (of each strategy) can also be tested in animal models using established and published protocols. This can provide information about the long-term efficacy of the CRISPR-Cas9 system in preventing HIV-1 infection and disease progression.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

As used herein, all headings are simply for organization and are not intended to limit the disclosure in any manner. The content of any individual section may be equally applicable to all sections.

### INCORPORATION BY REFERENCE

All patents and publications referenced herein are hereby incorporated by reference in their entireties, including published PCT application, WO 2020/227369, filed May 6, 2020, titled “Tailored Hypoimmune Nanovesicle Delivery Systems for Cancer Tumors,” and published U.S. non-provisional application, US 20220040106 A1, filed Aug. 3, 2021, titled “Tailored Hypoimmune Nanovesicular Delivery Systems for Cancer Tumors, Hereditary and Infectious Diseases.”

What is claimed is:

1. A biomimetic nanovesicle (BioNV), wherein the BioNV comprises a targeting agent that is targeted against at least a first cell surface marker of a latently HIV-infected cell, and wherein the BioNV is configured to deliver a gene editing payload comprising at least two guide RNAs (gRNAs).

2. A biomimetic nanovesicle (BioNV), wherein the BioNV comprises a targeting agent that is targeted against at least a first cell surface marker of a latently HIV-infected cell, and wherein the BioNV is configured to deliver:
a) a gene editing payload comprising at least two gRNAs; and
b) a small RNA.

3. The BioNV of claim 1 or 2, wherein the BioNV is derived from a hypoimmunogenic modified cell.

4. The BioNV of claim 3, and wherein the hypoimmunogenic modified cell is a stem cell, an induced pluripotent stem cell (iPSC), a reprogrammed pluripotent or multipotent cell, an embryonic stem cell, a mesenchymal stem cell, or a differentiated cell which originates from any modified cell thereof.

5. The BioNV of claim 4, wherein the hypoimmunogenic modified cell is an iPSC.

6. The BioNV of claim 4, wherein the hypoimmunogenic modified cell is a T cell, helper T cell, T-memory cell, γδ T cell, NK cell, monocyte, or macrophage.

7. The BioNV of any one of the preceding claims, wherein the BioNV substantially lacks one or more MHC class I proteins, MHC class II proteins, T cell receptor (TCR) proteins, and/or cytokine release syndrome (CRS) proteins.

8. The BioNV of any one of the preceding claims, wherein the BioNV has reduced or ablated β2-macroglobulin (B2M) protein expression and/or activity and/or MHC class I protein expression and/or activity.

9. The BioNV of any one of the preceding claims, wherein the BioNV has reduced or ablated CIITA protein expression and/or activity and/or MHC class II protein expression and/or activity.

10. The BioNV of any one of the preceding claims, wherein the BioNV has reduced or ablated human leukocyte antigen A (HLA-A) protein expression and/or activity.

11. The BioNV of any one of the preceding claims, wherein the BioNV has reduced or ablated human leukocyte antigen B (HLA-B) protein expression and/or activity.

12. The BioNV of any one of the preceding claims, wherein the BioNV has reduced or ablated human leukocyte antigen C (HLA-C) protein expression and/or activity.

13. The BioNV of any one of the preceding claims, wherein the BioNV has reduced or ablated human leukocyte antigen E (HLA-E) or human leukocyte antigen G (HLA-G) protein expression and/or activity.

14. The BioNV of any one of the preceding claims, wherein the BioNV has reduced or ablated human leukocyte antigen F (HLA-F) protein expression and/or activity.

15. The BioNV of any one of the preceding claims, wherein the BioNV has reduced or ablated T cell alpha constant (TRAC) protein expression and/or activity.

16. The BioNV of any one of the preceding claims, wherein the BioNV has reduced or ablated T cell beta constant (TRBC) protein expression and/or activity.

17. The BioNV of any one of the preceding claims, wherein the BioNV has reduced or ablated programmed cell death protein 1 (PD-1) expression and/or activity; or wherein the BioNV has PD-1 protein expression and/or activity.

18. The BioNV of any one of the preceding claims, wherein the BioNV has reduced or ablated interleukin-4 (IL-4) protein expression and/or activity.

19. The BioNV of any one of the preceding claims, wherein the BioNV has reduced or ablated interleukin-6 (IL-6) protein expression and/or activity.

20. The BioNV of any one of the preceding claims, wherein the BioNV has reduced or ablated interleukin-10 (IL-10) protein expression and/or activity.

21. The BioNV of any one of the preceding claims, wherein the BioNV has reduced or ablated interleukin-16 (IL-16) protein expression and/or activity.

22. The BioNV of any one of the preceding claims, wherein the BioNV has SerpinB9 protein expression and/or activity.

23. The BioNV of any one of the preceding claims, wherein the BioNV has CD34 protein expression and/or activity.

24. The BioNV of any one of the preceding claims, wherein the BioNV has CCL2 protein expression and/or activity.

25. The BioNV of any one of the preceding claims, wherein the BioNV has PD-L1 protein expression and/or activity.

26. The BioNV of any one of the preceding claims, wherein the BioNV has H2-M3 protein expression and/or activity.

27. The BioNV of any one of the preceding claims, wherein the BioNV has CD47 protein expression and/or activity.

28. The BioNV of any one of the preceding claims, wherein the BioNV has CD24 protein expression and/or activity.

29. The BioNV of any one of the preceding claims, wherein the BioNV has chimeric CD24/CD47 protein expression and/or activity.

30. The BioNV of any one of the preceding claims, wherein the BioNV has CD200 protein expression and/or activity.

31. The BioNV of any one of the preceding claims, wherein the BioNV has chimeric CD24/CD200 protein expression and/or activity or chimeric CD47/CD200 protein and/or activity.

32. The BioNV of any one of the preceding claims, wherein the BioNV has CTLA-4 protein expression and/or activity.

33. The BioNV of any one of the preceding claims, wherein the BioNV has MFG-E8 protein expression and/or activity.

34. The BioNV of any one of the preceding claims, wherein the BioNV has NCAM protein expression and/or activity.

35. The BioNV of any one of the preceding claims, wherein the BioNV has α-phagocytic integrin protein expression and/or activity.

36. The BioNV of any one of the preceding claims, wherein the BioNV has FasL protein expression and/or activity.

37. The BioNV of any one of the preceding claims, wherein the BioNV has reduced or ablated protein expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

38. The BioNV of any one of the preceding claims, wherein the BioNV has expression and/or activity of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

39. The BioNV of any one of the preceding claims, wherein the BioNV has reduced or ablated protein expression and/or activity of 3 or more immunogenic proteins, and expression and/or activity of 3 or more immunoprotective proteins.

40. The BioNV of any one of the preceding claims, wherein the BioNV is allogeneic.

41. The BioNV of any one of the preceding claims, wherein the BioNV does not cause an adverse immune reaction in subjects to which it is administered.

42. The BioNV of any one of the preceding claims, wherein the BioNV substantially lacks protein and/or activity of HLA-A, HLA-B, HLA-C, HLA-F, CIITA, IL-6, TRAC, TRBC, and one of either HLA-E or HLA-G.

43. The BioNV of any one of the preceding claims, wherein the BioNV substantially lacks protein and/or activity of HLA-A, HLA-B, HLA-C, HLA-F, CIITA, IL-6, TRAC, TRBC, PD-1, and one of either HLA-E or HLA-G.

44. The BioNV of any one of the preceding claims, wherein the BioNV substantially lacks protein and/or activity of HLA-A, HLA-B, HLA-C, HLA-F, CIITA, IL-6, TRAC, TRBC, PD-1, one of either HLA-E or HLA-G, and one or more of IL-4, IL-10, and IL-16.

45. The BioNV of any one of the preceding claims, wherein the BioNV has membrane-embedded α-phagocytic integrin, CCL2, H2-M3, FasL, MFEG8, and PD-L1 and/or CTLA-4; and
one of either CD24, CD47, CD200, chimeric CD24/CD47, chimeric CD24/CD200, and chimeric CD47/CD200, or two of either CD24, CD47, and CD200.

46. The BioNV of any one of the preceding claims, wherein the BioNV has membrane-embedded α-phagocytic integrin, CCL2, H2-M3, FasL, MFEG8, SerpinB9, and PD-L1 and/or CTLA-4; and
one of either CD24, CD47, CD200, chimeric CD24/CD47, chimeric CD24/CD200, and chimeric CD47/CD200, or two of either CD24, CD47, and CD200.

47. The BioNV of any one of the preceding claims, wherein the BioNV has membrane-embedded CD200 protein and substantially lacks protein of either CD24 or CD47.

48. The BioNV of any one of the preceding claims, wherein the targeting agent is one or more of a CAR, VERR, viral ligand, viral receptor, or an antibody or antibody format selected from a monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody or antibody format.

49. The method of claim 48, wherein the VERR or viral ligand is a gp120/gp41 complex.

50. The BioNV of claim 48, wherein the targeting agent is a scFv.

51. The BioNV of claim 48, wherein the targeting agent is a CAR.

52. The BioNV of claim 51, wherein the CAR comprises a transmembrane domain of or derived from CD28, CD3ζ, CD4, CD8α, ICOS, or fragment and/or combination thereof.

53. The BioNV of claim 51, wherein the CAR comprises an intracellular domain further comprising an intracellular signaling domain of a CD3ζ-chain and/or one or more co-stimulatory molecules, optionally selected from CD28, 4-1BB, ICOS, CD27, and OX40.

54. The BioNV of claim 51, wherein the CAR is activated, optionally wherein the CAR is activated via its target, through another receptor, and/or a virus.

55. The BioNV of any one of the preceding claims, wherein the first cell surface marker is or comprises a sialic acid-binding immunoglobulin-like lectin (Siglec) molecule.

56. The BioNV of claim 55, wherein the Siglec molecule is Siglec-1.

57. The BioNV of any one of claims 1-54, wherein the first cell surface marker is or comprises CD2, CD3, CD4, CCR5, CD20, CD25, CD30, CD32a, CD69, CD91, CD160, CD257, LAG-3, CD147, CD231, CEACAM1, PLXNB2, HLA-DR, PD-1, CTLA-4, TIGIT, LAG-3, TIM-3, BTK, Cyclophilin B, Sec62, Rab10, or SPCS.

58. The BioNV of any one of the preceding claims, wherein the gene editing payload comprises one or more gene editors.

59. The BioNV of claim 58, wherein the one or more gene editors forms a complex with at least one of the two gRNAs.

60. The BioNV of claim 58, wherein the one or more gene editors is a site-directed endonuclease, CRISPR/Cas nuclease, SpCas9-HF1, Cpf1, CasX, C2c1, C2c2, C2c3, Cas9, TevCas9, Archaea Cas9, CasY.1, CasY.2, CasY.3, CasY.4, CasY.5, CasY.6, Cas omega, transposase, and/or an ortholog or homolog thereof.

61. The BioNV of claim 60, wherein the gene editor is a CRISPR/Cas nuclease.

62. The BioNV of claim 60, wherein the gene editor is a site-directed endonuclease.

63. The BioNV of any one of claims 58-62, wherein the one or more gene editors excises the HIV proviral genomic sequence between the at least two gRNA target sequences in the latently HIV-infected cell.

64. The BioNV of claim 63, wherein the excision retains in the latently HIV-infected cell an integrated 5′ LTR sequence, TAR Loop sequence, and an at least a portion of a Gag coding sequence.

65. The BioNV of claim 63, wherein the excision retains in the latently HIV-infected cell an integrated 3′ LTR sequence and at least a portion of a Nef coding sequence.

66. The BioNV of any one of claim 63, wherein the excision retains in the latently HIV-infected cell at least a portion of an integrated 5′ LTR sequence, TAR Loop sequence, at least a portion of a Gag coding sequence, and at least a portion of a 3′ LTR sequence.

67. The BioNV of any one of claim 63, wherein the excision retains in the latently HIV-infected cell at least a portion of an integrated 5′ LTR sequence, TAR Loop sequence, at least a portion of a Gag coding sequence, at least a portion of a Nef coding sequence, at least a portion of a 3′ LTR sequence, and at least one stop codon downstream of the Gag coding sequence and upstream of the Nef coding sequence and at least one start codon downstream of the stop codon and upstream of the Nef coding sequence.

68. The BioNV of claim 63, wherein the excision disrupts an integrated HIV TAR loop sequence.

69. The BioNV of claim 63, wherein the excision retains in the latently HIV-infected cell at least a portion of an integrated 5′ LTR, at least a portion of an HIV Env sequence, and at least a portion of an HIV Nef sequence.

70. The BioNV of any one of the preceding claims, wherein the at least two gRNAs comprise:
a first gRNA complementary to a first coding sequence and/or a first non-coding sequence; and
a second gRNA complementary to a second coding sequence and/or a second non-coding sequence.

71. The BioNV of claim 70, wherein the first non-coding sequence is an integrated HIV TAR loop sequence.

72. The BioNV of claim 70, wherein the first non-coding sequence is 5′ of an integrated HIV TAR loop sequence and/or within a 5′ LTR sequence.

73. The BioNV of claim 70, wherein the first coding sequence is an HIV Gag proviral genomic sequence.

74. The BioNV of claim 73, wherein an excision site corresponding to the first coding sequence is at least about 21 base pairs (bp) downstream of the Gag protein start codon.

75. The BioNV of claim 70, wherein the second coding sequence and/or the second non-coding sequence is downstream of the first coding sequence and/or the first non-coding sequence and upstream of at least portion of an integrated HIV Env sequence.

76. The BioNV of claim 70, wherein the second coding sequence is an HIV Nef proviral genomic sequence, or wherein the second non-coding sequence is an HIV 3′ LTR genomic sequence.

77. The BioNV of claim 76, wherein an excision site corresponding to the second coding sequence is at least about 21 base pairs (bp) upstream of the Nef protein stop codon.

78. The BioNV of any one of the preceding claims, wherein the at least two gRNAs comprise:
one or more nucleic sequences selected from SEQ ID NOs: 1-20, or one or more nucleic acids comprising one or more substitutions, insertions, or deletions thereof, that is optionally compatible with Cas9 and optionally targets Nef;
one or more nucleic sequences selected from SEQ ID NOs: 21-31, or one or more nucleic acids comprising one or more substitutions, insertions, or deletions thereof, that is immunogenic proteins compatible with Cpf1/CasX and immunogenic proteins targets Nef;
one or more nucleic sequences selected from SEQ ID NOs: 32-51, or one or more nucleic acids comprising one or more substitutions, insertions, or deletions thereof, that is immunogenic proteins compatible with Cas9 and immunogenic proteins targets Pol;
one or more nucleic sequences selected from SEQ ID NOs: 52-71, or one or more nucleic acids comprising one or more substitutions, insertions, or deletions thereof, that is immunogenic proteins compatible with Cpf1/CasC and immunogenic proteins targets Pd;
one or more nucleic sequences selected from SEQ ID NOs: 72-91, or one or more nucleic acids comprising one or more substitutions, insertions, or deletions thereof, that is immunogenic proteins compatible with Cas9 and immunogenic proteins targets Rev;
one or more nucleic sequences selected from SEQ ID NOs:92-107, or one or more nucleic acids comprising one or more substitutions, insertions, or deletions thereof, that is immunogenic proteins compatible with Cpf1/CasX and immunogenic proteins targets Rev;
one or more nucleic sequences selected from SEQ ID NOs: 108-127, or one or more nucleic acids comprising one or more substitutions, insertions, or deletions thereof, that is immunogenic proteins compatible with Cas9 and immunogenic proteins targets Gag;
one or more nucleic sequences selected from SEQ ID NOs: 128-147, or one or more nucleic acids comprising one or more substitutions, insertions, or deletions thereof, that is immunogenic proteins compatible with Cpf1/CasX and immunogenic proteins targets Gag;
one or more nucleic sequences selected from SEQ ID NOs: 148-166, or one or more nucleic acids comprising one or more substitutions, insertions, or deletions thereof, that is immunogenic proteins compatible with Cas9 and immunogenic proteins targets Tat;
one or more nucleic sequences selected from SEQ ID NOs: 167-193, or one or more nucleic acids comprising one or more substitutions, insertions, or deletions thereof, that is immunogenic proteins compatible with Cas9 and immunogenic proteins targets a 5′LTR sequence of HXB2;
one or more nucleic sequences selected from SEQ ID NOs: 194-210, or one or more nucleic acids comprising one or more substitutions, insertions, or deletions thereof, that is immunogenic proteins compatible with Cpf1/CasX and immunogenic proteins targets a 5′LTR sequence of HXB2;
one or more nucleic sequences selected from SEQ ID NOs: 211-230, or one or more nucleic acids comprising one or more substitutions, insertions, or deletions thereof, that is immunogenic proteins compatible with Cas9 and immunogenic proteins targets a 3′LTR sequence of HXB2;
one or more nucleic sequences selected from SEQ ID NOs: 231-247, or one or more nucleic acids comprising one or more substitutions, insertions, or deletions thereof, that is immunogenic proteins compatible with Cpf1/CasX and immunogenic proteins targets a 3′LTR sequence of HXB2;
one or more nucleic sequences selected from SEQ ID NOs: 249-254, or one or more nucleic acids comprising one or more substitutions, insertions, or deletions thereof, that is immunogenic proteins compatible with Cas9 and immunogenic proteins targets HXB2 TAR; and/or
a nucleic sequence of SEQ ID NOs: 255, or a nucleic acid comprising one or more substitutions, insertions, or deletions thereof, that is immunogenic proteins compatible with Cpf1/CasX and immunogenic proteins targets HXB2 TAR.

79. The BioNV of any one of claims 2-78, wherein the small RNA targets an HIV coding sequence.

80. The BioNV of any one of claim 79, wherein the small RNA targets an HIV Gag coding sequence or an HIV Nef coding sequence.

81. The BioNV of claim 80, wherein the HIV Nef coding sequence is a portion of a messenger RNA encoding a Nef protein.

82. The BioNV of any one of claims 79-81, wherein the small RNA is configured to interact with an RNA-induced silencing complex (RISC) in the latently HIV-infected cell.

83. The BioNV of claim 82, wherein the interaction with the RISC complex induces silencing of the HIV Nef RNA sequence.

84. The BioNV of claim 82, wherein the interaction with the RISC complex induces amplification of the small RNA.

85. The BioNV of any one of claims 79-84, wherein the small RNA is configured to be trafficked from the latently HIV-infected cell to at least a second HIV-infected cell.

86. The BioNV of any one of claims 79-85, wherein the small RNA functions to prevent and/or reverse Nef-mediated multiple histocompatibility complex (MHC) sequestration in the latently HIV-infected cell and/or the second HIV-infected cell.

87. The BioNV of claim 85, wherein the trafficking is through a SIDT-1/2 cell surface protein complex.

88. The BioNV of any one of claims 79-86, wherein the small RNA is one or more of a tracer RNA (tracrRNA), micro RNA (miRNA), RNA inference (RNAI), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), and small non-coding RNA.

89. The BioNV of claim 88, wherein the small RNA is a RNAi.

90. The BioNV of claim 88, wherein the small RNA is a shRNA.

91. The BioNV of any one of the preceding claims, wherein the BioNV encapsulates one or more additional nucleic acids.

92. The BioNV of claim 91, wherein the one or more additional nucleic acids encodes one or more gene editing payloads.

93. The BioNV of claim 92, wherein the one or more gene editing payloads is a CRISPR endonuclease and/or site-directed endonuclease.

94. The BioNV of claim 91, wherein the one or more additional nucleic acids encodes one or more gRNA sequences.

95. The BioNV of claim 91, wherein the one or more additional nucleic acids encodes one or more small RNAs that targets an HIV sequence.

96. The BioNV of claim 91, wherein the one or more additional nucleic acids encodes an HIV Tat sequence.

97. The BioNV of claim 91, wherein the one or more additional nucleic acids encodes one or more aptamers configured to bind an HIV TAR sequence.

98. The BioNV of claim 91, wherein the one or more additional nucleic acids comprises one or more low-level expression constitutively active promoters, tissue-specific promoters, cell-specific promoters, and/or suicide promoters.

99. The BioNV of any one of the preceding claims, wherein the BioNV encapsulates one or more latency reversal agents (LRAs).

100. The BioNV of any one of the preceding claims, wherein the BioNV encapsulates one or more antiretroviral therapies.

101. The BioNV of claim 100, wherein the antiretroviral therapy is a non-nucleoside reverse transcriptase inhibitor (NNRTI), optionally selected from efavirenz (SUSTIVA), rilpivirine (EDURANT) and doravirine (PIFELTRO).

102. The BioNV of claim 100, wherein the antiretroviral therapy is a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY).

103. The BioNV of claim 100, wherein the antiretroviral therapy is a protease inhibitor (PI), optionally selected from atazanavir (REYATAZ), darunavir (PREZISTA), and lopinavir/ritonavir (KALETRA).

104. The BioNV of claim 100, wherein the antiretroviral therapy is an integrase inhibitor, optionally selected from bictegravir sodium/emtricitabine/tenofovir alafenamide fumar (BIKTARVY), raltegravir (ISENTRESS), and dolutegravir (TIVICAY).

105. The BioNV of claim 100, wherein the antiretroviral therapy is an entry or fusion inhibitor, optionally selected from enfuvirtide (FUZEON) and maraviroc (SELZENTRY).

106. The BioNV of any one of the preceding claims, wherein the BioNV is about 10 nm to about 1200 nm in size.

107. The BioNV of claim 106, wherein the BioNV is about 10 nm to about 100 nm in size.

108. The BioNV of claim 106, wherein the BioNV is about 100 nm to about 200 nm in size.

109. The BioNV of claim 106, wherein the BioNV is about 200 nm to about 500 nm in size.

110. The BioNV of claim 106, wherein the BioNV is about 500 nm to about 1200 in size.

111. The BioNV of any one of the preceding claims, wherein the BioNV is stored at about −80° C. or suitable for storage at about −80° C.

112. The BioNV of any one of the preceding claims, wherein the BioNV is lyophilized or suitable for lyophilization.

113. A method of treating an HIV infection comprising administering to a subject in need thereof a BioNV of any one of claims 1-112.

114. The method of claim 113, wherein the HIV is HIV-1 or HIV-2.

115. The method of claim 114, wherein the HIV-1 is one of Group M, Group N, Group O, and Group P.

116. The method of claim 114 or 115, wherein the HIV-1 is Group M and a subtype selected from subtype A, B, C, D, F, G, H, I, J, K, and L.

117. The method of any one of claims 113-116, wherein administering the BioNV results in delivery of at least two gRNAs and/or one or more gene editors.

118. The method of claim 117, wherein the one or more gene editors excises an HIV proviral genomic sequence between the at least two gRNA target sequences in the latently HIV-infected cell.

119. The method of claim 118, wherein the excision retains in the latently HIV-infected cell an integrated 5′ LTR sequence, TAR Loop sequence, and an at least a portion of a Gag coding sequence.

120. The method of claim 118, wherein the excision by the one or more gene editors retains in the latently HIV-infected cell an integrated 3′ LTR sequence and at least a portion of a Nef coding sequence.

121. The method of claim 118, wherein the excision retains an integrated 5′ LTR sequence, TAR Loop sequence, at least a portion of a Gag coding sequence, at least a portion of the Nef coding sequence, a 3′ LTR sequence, and at least one stop codon downstream of the Gag coding sequence and upstream of the Nef coding sequence and at least one start codon downstream of the stop codon and upstream of the Nef coding sequence.

122. The method of claim 118, wherein the excision retains an integrated 5′ LTR sequence, TAR Loop sequence, at least a portion of a Gag coding sequence, and a 3′ LTR sequence.

123. The BioNV of claim 118, wherein the excision disrupts an integrated HIV TAR loop sequence.

124. The BioNV of claim 118, wherein the excision retains in the latently HIV-infected cell at least a portion of an integrated 5′ LTR sequence, at least a portion of an HIV Env sequence, and at least a portion of an HIV Nef sequence.

125. The method of any one of claims 113-124, wherein administering the BioNV results in the expression of one or more HIV proteins or one or more portions thereof in an HIV-infected cell.

126. The method of claim 125, wherein the one or more HIV proteins is p1, p2, p6, p7, p17, p24, HIV polymerase, gp41, or gp120.

127. The method of claim 125 or 126, wherein the one or more HIV proteins is processed and presented via a multiple histocompatibility complex (MHC) in the latently HIV-infected cell.

128. The method of claim 113, wherein the BioNV delivers a small RNA.

129. The method of claim 113, wherein administering the BioNV results in expression of a partial Nef RNA (pNef).

130. The method of claim 128 or 129, wherein the pNef is targeted by the small RNA.

131. The method of any one of claims 128-130, wherein the small RNA functions to prevent and/or reverse Nef-mediated MHC sequestration in the latently HIV-infected cell.

132. The method of any one of claims 128-131, wherein the small RNA interacts with an RNA-induced silencing complex (RISC) in the latently HIV-infected cell.

133. The method of claim 132, wherein the interaction between the small RNA and the RISC in the latently HIV-infected cell results in amplification of the small RNA.

134. The method of any one of claims 128-133, wherein the small RNA is configured to be trafficked from the latently HIV-infected cell to at least a second HIV-infected cell.

135. The method of claim 134, wherein the trafficking is through a SIDT-1/2 cell surface protein complex in the latently HIV-infected cell.

136. The method of any one of claims 113-135, wherein administering the BioNV results in γδ T cell costimulation of NK cells in the subject.

137. The method of any one of claims 113-136, wherein administering the BioNV results in restoration of the γδ T cell population in the subject relative to the subject prior to the administration and/or relative to a subject not administered the BioNV.

138. The method of claim 136 or 137, wherein the γδ T cell is of a Vγ9JP repertoire.

139. The method of any one of claims 113-138, wherein administering the BioNV results in T cell-mediated cytotoxicity against one or more HIV-infected cells.

140. The method of any one of claims 113-139, wherein administering the BioNV results in expression of a Tat sequence.

141. The method of any one of claims 113-140, where administering the BioNV results in aptamer binding of a TAR sequence.

142. The method of any one of claims 113-141, wherein administering the BioNV results in delivery of one or more latency reversal agents (LRAs).

143. The method of any one of claims 113-142, wherein administering the BioNV results in delivery of one or more antiretroviral therapies.

144. The method of any one of claims 113-143, further comprising administering one or more antiretroviral therapies.

145. The method of claim 143 or 144, wherein the antiretroviral therapy is a non-nucleoside reverse transcriptase inhibitor (NNRTI), optionally selected from efavirenz (SUSTIVA), rilpivirine (EDURANT) and doravirine (PIFELTRO).

146. The method of claim 143 or 144, wherein the antiretroviral therapy is a nucleoside or nucleotide reverse transcriptase inhibitor (NRTI), optionally selected from abacavir (ZIAGEN), tenofovir (VIREAD), emtricitabine (EMTRIVA), lamivudine (EPIVIR), zidovudine (RETROVIR), emtricitabine/tenofovir (TRUVADA), and emtricitabine/tenofovir alafenamide (DESCOVY).

147. The method of claim 143 or 144, wherein the antiretroviral therapy is a protease inhibitor (PI), optionally selected from atazanavir (REYATAZ), darunavir (PREZISTA), and lopinavir/ritonavir (KALETRA).

148. The method of claim 143 or 144, wherein the antiretroviral therapy is an integrase inhibitor, optionally selected from bictegravir sodium/emtricitabine/tenofovir alafenamide fumar (BIKTARVY), raltegravir (ISENTRESS), and dolutegravir (TIVICAY).

149. The method of claim 143 or 144, wherein the antiretroviral therapy is an entry or fusion inhibitor, optionally selected from enfuvirtide (FUZEON) and maraviroc (SELZENTRY).

150. The method of any one of claims 143-149, wherein the antiretroviral therapy comprises at least two, or at least three antiretroviral therapies, or at least four of the antiretroviral therapies in combination.

151. The method of any one of claims 113-150, wherein administering the BioNV comprises providing one or more doses of at least about 1 ng/kg to at least about 10 mg/kg of BioNVs.

152. The method of any one of claims 113-151, wherein the administration is intravenous, intramuscular, or parenteral.

153. A method of treating an HIV infection in a subject comprising:
administering to the subject a biomimetic nanovesicle (BioNV), wherein the BioNV comprises a targeting moiety that is targeted against at least a first cell surface marker of a latently HIV-infected cell, and wherein the BioNV is configured to deliver:
a) a gene editing payload comprising at least two gRNAs, wherein the at least two gRNAs comprise:
i) a first gRNA targeted against an HIV Gag proviral genomic sequence; and
ii) a second gRNA targeted against an HIV proviral 3′ LTR genomic sequence,
wherein the gene editing payload is capable of excising the HIV proviral genomic sequence between the HIV Gag proviral genomic sequence and the HIV 3′ LTR proviral genomic sequence, and
wherein an HIV 5′ LTR sequence, TAR Loop sequence, at least a portion of the HIV Gag proviral genomic sequence, and at least a portion of the HIV 3′ LTR proviral genomic sequence is not excised from the latently HIV-infected cell.

154. A method of treating an HIV infection in a subject comprising:
administering to the subject a biomimetic nanovesicle (BioNV), wherein the BioNV comprises a targeting moiety that is targeted against at least a first cell surface marker of a latently HIV-infected cell, and wherein the BioNV is configured to deliver:
a) a gene editing payload comprising at least two gRNAs, wherein the at least two gRNAs comprise:
i) a first gRNA targeted against an HIV Gag proviral genomic sequence; and
ii) a second gRNA targeted against an HIV proviral Nef genomic sequence,
wherein the gene editing payload is capable of excising the HIV proviral genomic sequence between the HIV Gag proviral genomic sequence and the HIV Nef proviral genomic sequence, and
wherein an HIV 5′ LTR sequence, TAR Loop sequence, at least a portion of the HIV Gag proviral genomic sequence, and at least a portion of the HIV Nef proviral genomic sequence is not excised from the latently HIV-infected cell; and
b) an interference RNA (RNAi), wherein the RNAi is targeted against a messenger RNA sequence complementary to the portion of the HIV Nef proviral genomic sequence that is not excised.

155. A method of treating an HIV infection in a subject comprising:
administering to the subject a biomimetic nanovesicle (BioNV), wherein the BioNV comprises a targeting moiety that is targeted against at least a first cell surface marker of a latently HIV-infected cell, and wherein the BioNV is configured to deliver:
a) a gene editing payload comprising at least two gRNAs, wherein the at least two gRNAs comprise:
i) a first gRNA targeted against an HIV Gag proviral genomic sequence; and
ii) a second gRNA targeted against an HIV Nef proviral genomic sequence,
wherein the gene editing payload is capable of excising the HIV proviral genomic sequence between the HIV Gag proviral genomic sequence and the HIV Nef proviral genomic sequence, and
wherein at least a portion of a 5′ end of the HIV proviral genomic sequence and a portion of a 3′ end of the HIV proviral genomic sequence is not excised from the latently HIV-infected cell; and
b) an interference RNA (RNAi), wherein the RNAi is targeted against a messenger RNA sequence complementary to the portion of the HIV Nef proviral genomic sequence that is not excised,
wherein the latently HIV-infected cell expresses a partial Nef RNA sequence (pNef) from the portion of the HIV Nef proviral genomic sequence which reduces Nef-mediated MHC complex sequestration in the latently HIV-infected cell,
wherein the latently HIV-infected cell expresses a partial Gag protein (pGag) from the portion of the portion of the HIV Gag proviral genomic sequence which is recognized by the MHC complex and results in T-cell stimulation against the latently HIV-infected cell, and
wherein the pNef and pGag expression in the latently HIV-infected cell results in:
a) processing and presentation of the pGag via an MHC;
b) cell-to-cell spread of the RNAi via the SIDT-1/2 complex; and
c) T-cell mediated cytotoxicity against one or more latently HIV-infected cells.

156. A method of treating an HIV infection in a subject comprising:
administering to the subject a biomimetic nanovesicle (BioNV), wherein the BioNV comprises a targeting moiety that is targeted against at least a first cell surface marker of a latently HIV-infected cell, and wherein the BioNV is configured to deliver:
a) a gene editing payload comprising at least two gRNAs, wherein the at least two gRNAs comprise:
i) a first gRNA targeted against an HIV proviral genomic sequence within the TAR loop or 5′ of the TAR loop; and
ii) a second gRNA targeted against upstream of at least a portion of an HIV Env proviral genomic sequence,
wherein the gene editing payload is capable of excising the HIV proviral genomic sequence between the first gRNA target sequence and the second gRNA target sequence, and
wherein at least a portion of a 5′ end of the HIV proviral genomic sequence and a portion of the HIV Env proviral genomic sequence is not excised from the latently HIV-infected cell; and
b) an interference RNA (RNAi), wherein the RNAi is targeted against a messenger RNA sequence complementary to a portion of an HIV Nef proviral genomic sequence that is not excised,
wherein the latently HIV-infected cell expresses a partial Nef RNA sequence (pNef) from the portion of the HIV Nef proviral genomic sequence which reduces Nef-mediated MHC complex sequestration in the latently HIV-infected cell,
wherein the latently HIV-infected cell expresses a partial gp120 and/or gp41 peptide from the portion of the HIV Env proviral genomic sequence that is not excised, which is recognized by the MHC complex and results in T-cell stimulation against the latently HIV-infected cell, and
wherein the pNef and gp120/gp41 expression in the latently HIV-infected cell results in:
a) processing and presentation of gp120/gp41 peptides via an MHC;
b) cell-to-cell spread of the RNAi via the SIDT-1/2 complex; and
c) T-cell mediated cytotoxicity against one or more latently HIV-infected cells.

157. A method of treating an HIV infection in a subject comprising:
administering to the subject a biomimetic nanovesicle (BioNV), wherein the BioNV comprises a targeting moiety that is targeted against at least a first cell surface marker of a latently HIV-infected cell, and wherein the BioNV is configured to deliver:
a) a gene editing payload comprising at least two gRNAs, wherein the at least two gRNAs comprise:
i) a first gRNA targeted against an HIV Gag proviral genomic sequence; and
ii) a second gRNA targeted against upstream of at least a portion of an HIV Env proviral genomic sequence,
wherein the gene editing payload is capable of excising the HIV proviral genomic sequence between the first gRNA target sequence and the second gRNA target sequence, and
wherein at least a portion of an HIV TAR loop and 5′ LTR proviral genomic sequence and at least a portion of the HIV Env proviral genomic sequence is not excised from the latently HIV-infected cell; and
b) an interference RNA (RNAi), wherein the RNAi is targeted against a messenger RNA sequence complementary to a portion of an HIV Nef proviral genomic sequence that is not excised,
wherein the latently HIV-infected cell expresses a partial Nef RNA sequence (pNef) from the portion of the HIV Nef proviral genomic sequence which reduces Nef-mediated MHC complex sequestration in the latently HIV-infected cell,
wherein the latently HIV-infected cell expresses a partial gp120 and/or gp41 peptide from the portion of the HIV Env proviral genomic sequence that is not excised, which is recognized by the MHC complex and results in T-cell stimulation against the latently HIV-infected cell, and
wherein the pNef and gp120/gp41 expression in the latently HIV-infected cell results in:
a) processing and presentation of gp120/gp41 peptides via an MHC;
b) cell-to-cell spread of the RNAi via the SIDT-1/2 complex; and
c) T-cell mediated cytotoxicity against one or more latently HIV-infected cells.
